SEQUENCE LISTING (13 sequences)

SEQ ID NO: 1 - protein, 2003 aa, Homo sapiens
  ref <308>: DATABASE ACCESSION NUMBER: Q99466
  ref <309>: DATABASE ENTRY DATE: 2021-09-29
  ref <313>: RELEVANT RESIDUES IN SEQ ID NO: (1)..(2003)
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 2 - protein, 5 aa, Artificial Sequence
  Notch-4 antibody sequence
SYGMS

SEQ ID NO: 3 - protein, 10 aa, Artificial Sequence
  Notch-4 antibody sequence
GFTESSYGMS

SEQ ID NO: 4 - protein, 17 aa, Artificial Sequence
  Notch-4 antibody sequence
TINSNGGRTYYPDSVKG

SEQ ID NO: 5 - protein, 10 aa, Artificial Sequence
  Notch-4 antibody sequence
TINSNGGRTY

SEQ ID NO: 6 - protein, 6 aa, Artificial Sequence
  Notch-4 antibody sequence
DQGFAY

SEQ ID NO: 7 - protein, 11 aa, Artificial Sequence
  Notch-4 antibody sequence
KASQDVGTAVA

SEQ ID NO: 8 - protein, 7 aa, Artificial Sequence
  Notch-4 antibody sequence
WASTRHT

SEQ ID NO: 9 - protein, 9 aa, Artificial Sequence
  Notch-4 antibody sequence
QQYSSYPWT

SEQ ID NO: 10 - protein, 115 aa, Artificial Sequence
  Notch-4 antibody sequence
EVQLVESGGGLVQPGGSLKLSCAASGFTFSSYGMSWVRQTPDKRLELVATINSNGGRTYYPDSVKGRFTISRDNAKNTLYLQMSSLKSEDTAMYYCARDQGFAYWGQGTLVTVSA

SEQ ID NO: 11 - protein, 107 aa, Artificial Sequence
  Notch-4 antibody sequence
DIVMTQSHKFMSTSVGDRVSITCKASQDVGTAVAWDIVMTQSHKFMSTSVGDRVSITCKASQDVGTAVAWFTLTISNVQSEDLADYFCQQYSSYPWTFGGGTKLEIK

SEQ ID NO: 12 - DNA, 12 bp, Artificial Sequence
  sgRNA recognition sequence
gacgaaacaccg

SEQ ID NO: 13 - DNA, 12 bp, Artificial Sequence
  sgRNA recognition sequence
gtttaagagcta

(19) United States
(12) Patent Application Publication
(10) Pub. No.: US 2024/0043539 A1
(43) Pub. Date: Feb. 8, 2024

(54) METHODS OF INDUCING AN IMMUNOMODULATORY TUMOR RESPONSE

(71) Applicant: CORNELL UNIVERSITY, Ithaca, NY (US)

(72) Inventors: Dan-Avi LANDAU, New York, NY (US); Nir BEN-CHETRIT, New York, NY (US)

(21) Appl. No.: 18/265,796

(22) PCT Filed: Dec. 8, 2021
(86) PCT No.: PCT/US2021/062388

§ 371 Date: Jun. 7, 2023

Related U.S. Application Data
(60) Provisional application No. 63/122,815, filed on Dec. 8, 2020.

(51) Int. Cl. C07K 16/28; A61K 45/06; C12N 5/077; A61P 35/00

(52) U.S. Cl. CPC C07K 16/2818; A61K 45/06; C07K 16/2827; C07K 16/2878; C12N 5/0656; A61P 35/00

(57) ABSTRACT

The present disclosure relates to methods of inducing an immunomodulatory tumor response for the treatment of a subject having a tumor. The disclosure further relates to an organotypic tumor micro environment culture system that can be utilized to screen and identify novel immunomodulatory cancer therapeutics.

This application claims the priority benefit of U.S. Provisional Patent Application Ser. No. 63/122,815, filed Dec. 8, 2020, which is hereby incorporated by reference in its entirety.

### FIELD

The present disclosure relates to methods of inducing an immunomodulatory tumor response for the treatment of a subject having a tumor. The disclosure further relates to an organotypic tumor microenvironment culture system that can be utilized to screen and identify novel immunomodulatory cancer therapeutics.

### BACKGROUND

Tumors rely on complex interactions between malignant and non-malignant stromal and immune cells to create a tumor microenvironment (TME) niche that promotes tumor growth and enhances immune evasion (Hanahan and Coussens, “Accessories to the Crime: Functions of Cells Recruited to the Tumor Microenvironment,” Cancer Cell 21:309-322 (2012) and Quail and Joyce, “Microenvironmental Regulation of Tumor Progression and Metastasis,” Nat. Med. 19:1423-1437 (2013)). In breast cancer, infiltrating macrophages represent a major immune component of the TME Cassetta and Pollard, “Targeting Macrophages: Therapeutic Approaches in Cancer,” Nat. Rev. Drug Discov. (2018). During the growth of mammary tumors, macrophages accumulate significantly and undergo phenotypic alterations to enhance invasive growth, matrix remodeling, angiogenesis, and immune suppression (DeNardo et al., “Macrophages as Regulators of Tumour Immunity and Immunotherapy,” Nat. Rev. Immunology 19:369-382 (2019); Lin et al., “Macrophages Regulate the Angiogenic Switch in a Mouse Model of Breast Cancer,” Cancer Res. 66:11238-11246 (2006). Consistent with their pro-tumorigenic phenotypes, the accumulation of macrophages in breast cancer patients is generally associated with poor prognosis, resistance to therapies, and disease recurrence (DeNardo et al., “Leukocyte Complexity Predicts Breast Cancer Survival and Functionally Regulates Response To Chemotherapy,” Cancer Discov. 1:54-67 (2011); De Palma and Lewis, “Macrophage Regulation of Tumor Responses to Anticancer Therapies,” Cancer Cell 23:277-286 (2013); and Shabo et al., “Breast Cancer Expression of CD163, a Macrophage Scavenger Receptor, is Related to Early Distant Recurrence and Reduced Patient Survival,” Int. J. Cancer 123:780-786 (2008)).

These tumor-supporting characteristics have highlighted macrophages as a promising target for therapeutic intervention primarily through CSF-1R targeting (Cassetta and Pollard, “Targeting Macrophages: Therapeutic Approaches in Cancer,” Nat. Rev. Drug Discov. (2018)). Consistent with their pro-angiogenic and immunosuppressive roles, depletion of macrophages in mouse tumor models resulted in reduced tumor vascularization (Keklikoglou et al., “Periostin Limits Tumor Response to VEGFA Inhibition,” Cell Rep. 22:2530-2540 (2018)), increased intratumoral influx of cytotoxic CD8+ T and NK cells (Ries et al., “Targeting Tumor-Associated Macrophages with Anti-CSF-1R Antibody Reveals a Strategy for Cancer Therapy,” Cancer Cell 25:846-859 (2014)), and reduction of tumor burden (Qian et al., “CCL2 Recruits Inflammatory Monocytes to Facilitate Breast-Tumour Metastasis,” Nature 475:222-225 (2011); Zhu et al., “CSF1/CSF1R Blockade Reprograms Tumor-Infiltrating Macrophages and Improves Response to T-Cell Checkpoint Immunotherapy in Pancreatic Cancer Models,” Cancer Res. 74:5057-5069 (2014)). Nonetheless, sustained macrophage depletion strategies have safety concerns, especially when employed systemically, because macrophages are critical for homeostasis. The cessation of certain macrophage-targeting interventions resulted in a significant metastatic resurgence in pre-clinical models, as in the case of CCL2 inhibition (Bonapace et al., “Cessation of CCL2 Inhibition Accelerates Breast Cancer Metastasis by Promoting Angiogenesis,” Nature 515:130-133 (2014)). These limitations suggest that rather than eliminating macrophages or blocking their recruitment, they need to be selectively targeted their tumor-promoting phenotypes (Beatty et al., “CD40 Agonists Alter Tumor Stroma and Show Efficacy Against Pancreatic Carcinoma in Mice And Humans,” Science 331:1612-1616 (2011); Pyonteck et al., “CSF-1R Inhibition Alters Macrophage Polarization and Blocks Glioma Progression,” Nat. Med. 19:1264-1272 (2013); and Tseng et al., “Anti-CD47 Antibody-Mediated Phagocytosis of Cancer by Macrophages Primes an Effective Antitumor T-Cell Response,” Proc. Natl. Acad. Sci. U.S.A. 110:11103-11108 (2013)).

### SUMMARY

A first aspect of the present disclosure involves a method of inhibiting an immunosuppressive phenotype in a population of macrophages. This method involves administering to a population of macrophages, an agent selected from a cyclin-dependent kinase 4 (Cdk4) inhibitor, a tumor necrosis factor related apoptosis-inducing ligand receptor 2 (TRAIL-R2) inhibitor, a protein tyrosine kinase 2 beta (Ptk2b) inhibitor, and a Notch-4 inhibitor under conditions effective to inhibit the immunosuppressive phenotype in the population of macrophages.

Another aspect of the present disclosure relates to a method of inhibiting macrophage proliferation in a population of cells comprising macrophages. This method involves administering a Notch-4 inhibitor to the population of cells under conditions effective to inhibit macrophage proliferation in a population of cells.

Another aspect of the present disclosure relates to a method of treating a tumor in a subject. This method involves administering, to a subject having a tumor, a Notch-4 inhibitor, where administering induces an anti-tumor immune response in the subject.

Another aspect of the present disclosure relates to a combination therapeutic involves a Notch-4 inhibitor and a checkpoint inhibitor.

Another aspect of the present disclosure relates to a combination therapeutic involves a Notch-4 inhibitor and a pro-inflammatory agent. Another aspect of the present disclosure relates to an organotypic tumor

microenvironment model (TME) culture system. The system involves an isolated population of cells, said population comprising tumor epithelial cells, mesenchymal stromal cells, and macrophages.

Identification of signals that drive macrophages toward pro-tumorigenic phenotypes (macrophage education) in non-scalable systems (such as tumor mouse models) poses a formidable barrier to target discovery through high-throughput screens. Therefore, the inventors developed a scalable organotypic TME (oTME) model from a murine mammary tumor (Lin et al., “Progression to Malignancy in the Polyoma Middle T Oncoprotein Mouse Breast Cancer Model Provides a Reliable Model for Human Diseases,” Am. J. Pathol. 163:2113-2126 (2003), which is hereby incorporated by reference in its entirety) that contains tumor epithelial cells and their supporting stromal cells and enables the application of high-throughput discovery platforms. The culture of macrophages in this system closely recapitulates their alteration toward pro-tumorigenic phenotype in vivo, via complex cellular interactions with tumor epithelial cells and stromal fibroblasts. The oTME platform was leveraged to dissect the macrophage education mechanisms using a genome-wide CRISPR/Cas9 screen in primary macrophages. The induction of Arginase-1 (Arg1) was utilized as a surrogate for educated macrophages and identified gene targets, including Cdk4 and Ptk2b, as druggable regulators that prevented the accumulation of Arg1+ macrophages. Second, using single-cell RNA-seq macrophage education time course, it was discovered that acquisition of proliferative and immunomodulatory phenotypes in macrophages followed an earlier transient activation of type-I interferons/STING that triggered proliferation in a subset of F4/80^{high}Ly6A+ macrophages that interact with stromal fibroblasts. Furthermore, it was demonstrated that macrophage localization and intra-cellular interactions in TME determined their phenotype. Using the murine model and human breast cancer specimens, macrophage-stroma interactions were found to give rise to proliferative, immunosuppressive and phagocytic phenotypes, while macrophage-tumor epithelial interactions resulted in pro-inflammatory phenotype. Finally, the Notch4 was identified as a targetable regulator of macrophage proliferation in pre-clinical models of breast cancer that effectively blocked tumor progression.

### BRIEF DESCRIPTION OF THE DRAWINGS

FIGS. 1A-1H shows breast organotypic TME model of cell-cell interactions of macrophages with tumor epithelial and stromal cells. Figure lA shows a scheme of isolation of tumor epithelial and stromal-like cells from MMTV-PyMT breast tumor (99LN parental cells; termed organotypic (o)TME). FIG. 1B shows an immunofluorescence of EpCAM (tumor epithelial), PDGFRA (stroma), and F4/80 (macrophages) demonstrating the typical cellular organization in intact MMTV-PyMT mammary tumor. Scale bars, 50pm. Representative images from 3 independent experiments. FIG. 1C shows an immunofluorescence for EPCAM and PDGFRA demonstrating the recapitulation of a typical tumor/stroma self-organization (as in FIG. 1B) in a two-week-old organotypic TME cultures. Data are representative of at least 5 independent experiments. Scale bars, 100 μm. FIG. 1D shows a representative flow cytometry and gating strategy of oTME cells using antibodies against EpCAM, PDGFRA, CD49f, CD29, CD24 and CD61. FIG. 1E shows immunofluorescence of macrophages in organotypic TME. Purified mTmG (Cyan) BM monocytes (n=3 replicates) were plated with oTME cells and seven days later stained for E-cadherin and PDGFRA. Scale bars, 100 μm. Macrophages were scored according to their spatial association with E-cadherin+ or PDGFRA+ cells. Data (fields of view; n=15) are representative of 3 independent experiments and shown as mean±SD., 2-tailed paired Student's t-test. FIG. 1F shows H&E and immunohistochemistry (IHC) of murine (IBA1) and human (CD163) breast cancer macrophages, demonstrating their spatial distribution in tumors. Scale bars, 100 μm. FIG. 1D shows a Volcano plot showing the fold change (x axis, log scale) and the FDR corrected p value (y axis, −log10 scale) of genes (n=21721) between M-CSF-treated BMDMs and BMDMs co-cultured with oTME cells for 7-days (n=3 replicates). Genes are dotted and colored by log fold change levels (color scale), the size of each dot represents the mean expression level (log scale). Vertical dotted lines represent ±1 log fold change. Horizontal dotted lines represent p value of 0.01 (−log10 scale). Selected genes are indicated by text. P values of zero were set to the smallest non-zero p value for visualization. FIG. 1H shows an immunophenotyping of BMDMs showing the impact of oTME education. BMDMs were co-cultured with oTME cells for 7-days and analyzed by flow cytometry.

FIGS. 2A-2F show the characterization of TME cellular components. FIG. 2A is the quantification and phase images of cell-type and their relative abundances by flow (n=5). FIG. 2B depicts qPCR of E-cadherin (Cdhl), Vimentin (Vim) and Pymt transgene, normalized to CD24-int mesenchymal cells (n=4 replicates). Data are shown as mean±SE. FIG. 2C shows mammary tumor growth of EpCAM+or EpCAM- orthotopic transplants in C57BL/6 WT mice (n=5 replicates). Tumor cells were isolated from liver and lung metastases (H&E staining of lungs metastasis) and analyzed for EpCAM and PDGFRA expression by flow cytometry. FIG. 2D depicts contact inhibition measured by EdU incorporation (following lhr exposure) in confluent cultures of oTME, EpCAM+ and PDGFRA+ sorted cells. Data (fields of view; oTME, n=13; EpCAM+n=10; PDGFRA+n=16) are shown as mean±SD., 2-way ANOVA test, Bonferroni-corrected. FIG. 2E shows in situ differentiation of BM Ly6C+ monocytes in the organotypic TME model. Purified BM monocytes from mTmG mice were allowed to differentiate for 7 days in the presence of oTME cells and scored for mT, CD45, Ly6C, CD11b, F4/80 expression by flow cytometry. Representative data from at least 5 independent experiments. FIG. 2F is a Gene Set Enrichment Analysis of Gene Ontology showing the significantly enriched (FDR<0.05) GO terms (top, GO term enrichment scores; bottom, rank positions of member genes in the GO term).

FIGS. 3A-3H show the time course single-cell RNAseq analysis of macrophage TME-education. FIG. 3A shows the Study design: BMDMs were co-cultured with oTME cells for 2 and 10-days or left unperturbed with M-CSF as control. Cells were profiled after 2 and 10 days with the 10× Chromium single-cell RNA-seq platform. FIG. 3B shows two-dimensional t-SNE plots (Diml and Dim2) of annotated single-cell transcriptomes colored by cell type (left) and time point (right). FIG. 3C shows two-dimensional t-SNE plot (left; Diml and Dim2) of macrophages (excluding M-CSF-treated day-10) and two-dimensional education trajectory (right; DM1 and DM2) with inferred pseudo-time axis (black line). The figure legend is the same as FIG. 3B. FIG. 3D is a heatmap showing macrophage education expression dynamics of M-CSF-treated (n=214), early (day 2, n=235), late (day 10, n=301) genes. The cells are sorted and split into 20 equal-width bins by the education pseudo-time, average expression level (row-wise Z-score; color scale) of education signature genes (rows) across each bin (columns) is shown. The figure legend is the same as FIG. 3B. FIG. 3E shows a Volcano plot showing log fold change (logFC) of genes (n=12322) between early (day-2) vs late (day-10) non-cycling educated macrophages (x axis) and their significance (y axis; -log10 scale). Genes are dotted and colored by logFC levels (color scale), the size of each dot represents the difference in the fraction of detection between the two groups. The p values were determined by Wilcoxon Rank Sum test. Vertical dotted lines represent ±0.75 logFC. Horizontal dotted lines represent p value of 0.01 (−log10 scale). Gene Set Enrichment Analysis in day-2 macrophages shows significant enrichment of Interferon alpha response (FDR<0.05). FIG. 3F shows t-SNE (left; Dim1 and Dim2) and violin plots (right) showing the clustering results of single-cell transcriptomes from educated and M-CSF-treated macrophages. FIG. 3G shows the expression of Irf7, Mki67 and Arg1 across education pseudo-time from M-CSF-treated, early to late educated macrophages. Each dot represents an average of cells in 50 equal-width bins on the education pseudo-time axis, same as FIG. 3E. Expressions are colored by the average logTPM levels (color scale), the size of each dot represents the fraction of detection in each bin. FIG. 3D shows BMDMs that were plated with oTME cells for 48 hrs in the presence of and EdU (10 μM), Tyk2 inhibitors (BMS-986165, 10 μM) or DMSO as control. EdU incorporation was analyzed by flow cytometry (n=4 replicates). Data are shown as mean±SD., 2-tailed unpaired Student's t-test.

FIGS. 4A-4C show the projection of ex vivo TME-education scRNA-seq signatures in human breast cancer macrophages. FIG. 4A shows t-SNE and violin plots of annotated single-cell transcriptomes and individual genes colored by cell lineage markers; Acta2+ (CD24^{Neg} stromal-like cells), Epcam+ (tumor epithelial cells), Cd24a+ and Epcam- (basal-like/mesenchymal cells), and Cd68 (macrophages). FIG. 4B shows macrophage ex vivo education pseudo-time from M-CSF-treated, 2-and-10 days educated macrophages. Wilcoxon Rank Sum test. FIG. 4C shows the projection of ex vivo education-signature (at day 10) on the human “M2-signature” as described by Azizi et al., “Single-Cell Map of Diverse Immune Phenotypes in the Breast Tumor Microenvironment,” Cell 174(36):1293-1308 (2018), which is hereby incorporated by reference in its entirety, using scRNA-seq data from human breast cancer immune cells. The murine ex vivo education-signature score in human myeloid and human tumor-associated macrophages (TAMs; subpopulation (left panel)). Principal component analysis of myeloid and TAM cells (middle panel). Ex vivo M2-signature score in myeloid and three TAMs subpopulations of human breast cancer: TAM 25, TAM 23, TAM 28. The left-to-right ordering represents the least-to-most M2-activation status according to (Azizi et al., “Single-Cell Map of Diverse Immune Phenotypes in the Breast Tumor Microenvironment,” Cell 174(36):1293-1308 (2018), which is hereby incorporated by reference in its entirety) (right panel). Wilcoxon Rank Sum test.

FIGS. 5A-5E show optimizations of CRISPR/cas9 screen in primary macrophages. FIG. 5A shows flow cytometry of EYFP induction in Arg1-EYFP BMDMs co-cultured with oTME cells for 7 days (oTME) or treated with M-CSF as control. FIG. 5B shows a CFSE proliferation assay in CD8 T-cells to determine immunosuppressive activity of oTME and M-CSF-treated BMDMs. Splenic T-cells were stimulated with CD3/CD28 antibodies, plated as indicated, and analyzed by flow cytometry 5-days later (n=6 replicates). Bars show mean±SD., 2-way ANOVA test, Bonferroni-corrected. FIG. 5C shows targeted sequencing of sgRNA cassettes quantifying abundances and frequencies in genome-wide CRISPR libraries. FIG. 5D shows gating strategy for FACS-isolation of Arg1-EYFP positive and Arg1-EYFP negative BMDMs in M2-education screen. FIG. 5E depicts the projection of an established gene module associated with CDK4/6 inhibition (Abemaciclib) in bulk RNAseq data from M-CSF-treated or educated macrophages.

FIGS. 6A-6H show a genome-wide pooled M2-like education screen in primary macrophages. (A) FIG. 6A shows the design for whole-genome CRISPR/Cas9 screen in Arg1-EYFP primary macrophages. FIG. 6B shows FACS-sorting of top 10% of EYFP+ and EYFP^{neg} BMDMs following 10 days of co-culture with oTME cells. FIG. 6C shows a Volcano plot of the CRISPR/Cas9 screen results. The x-axis shows log2 fold-change (LFC) of sgRNA abundance in EYFP^{neg} vs EYFP^{neg} BMDMs, and the y-axis shows the p-value calculated by MAGeCK package. Gene hits in EYFP″g BMDMs are highlighted in red. Distribution of LFC values (red lines) across all four sgRNAs of select gene targets (right). FIG. 6D shows the expression of screen gene targets from Arg1-EYFP^{neg} macrophages in bulk RNA-seq from M-CSF-treated and oTME educated BMDMs (Row-wise Z-score; color scale). FIG. 6E shows the effect of CDK4/6 inhibition (Abemaciclib) on Arg1-EYFP expression of oTME-educated macrophages. Arg1-EYFP BMDMs were co-cultured with oTME cells for 7 days, and then were treated with Abemaciclib (5, 10 μM) or DMSO for additional 7 days and analyzed by flow cytometry for EYFP (n=4 replicates). Data are shown as mean±SD., 2-tailed unpaired Student's t-test. FIG. 6F shows the effect of Abemaciclib on Arg1-EYFP expression in macrophages. Arg1-EYFP BMDMs were treated for 7 days with a cytokines cocktail consists of IL-4 (10 ng/mL), IL-13(80ng/mL), M-CSF(10 ng/mL) or with M-CSF alone as control (supplemented every 3 days). Cells were then treated with Abemaciclib (5 μM) or DMSO for additional 7 days and analyzed by flow cytometry for EYFP (n=3 replicates). Data are shown as mean±SD., 2-tailed unpaired Student's t-test. FIG. 6G shows the effect of PTK2B inhibition (PF-431369 on Arg1-EYFP expression of oTME-educated macrophages. Arg1-EYFP BMDMs were co-cultured with oTME cells for 7 days, and then were treated with PF-431369; (10 μM) or DMSO for additional 7 days and analyzed by flow cytometry for EYFP (n=4 replicates). Data are shown as mean±SD., 2-tailed unpaired Student's t-test. FIG. 6H shows the Effect of PF-431369 on Arg1 expression in macrophages. BMDMs were treated for 7 days with a cytokines cocktail consists of IL-4 (10 ng/mL), IL-13 (80 ng/mL), M-CSF (10 ng/mL) or with M-CSF alone as control (supplemented every 3 days). Cells then treated with PF-431369 (5 uM) or DMSO for additional 7 days. On day 14, cells were fixed and stained for Arg1 and CD206 and analyzed by flow cytometry. (n=3 replicates). Data are shown as mean±SD., 2-tailed unpaired Student's t-test.

FIGS. 7A-7C shows the proliferation dynamics of oTME macrophages during education FIG. 7A shows a stacked bar graph showing the composition (fraction) of each macrophage cluster to the corresponding conditions. FIG. 7B shows changes in relative proportions of cycling cells during macrophage education according to scRNA-seq clustering (n=1866 M-CSF-treated Day 2; n=1705 M-CSF-treated Day 10; n=145 Educated Day 2; n=552 Educated Day 10). Two-proportions Z-test, N.S. (not significant). FIG. 7C shows validation of cycling dynamics as depicted in (B) using EdU incorporation (n=3 replicates); data are shown as mean±SD., 2-tailed unpaired Student's t-test.

FIGS. 8A-8C shows the induction of Ly6A (Sca-1) in oTME and mammary tumor macrophages. (FIGS. 8A-8B) show flow cytometry of Ly6A (Sca-1) in oTME in A, mammary tumor and healthy glands macrophages in B. Histograms (FIG. 8B; right) show Ly6A (Sca-1) signal and FMO controls staining. FIG. 8C shows profiling of secreted proteins by cytokine arrays. Conditioned media were collected from FACS-purified tumor epithelial cells, stromal-like cells, and 7-day old oTME cell cultures were loaded and analyzed on cytokine arrays. The complete scans of cytokine arrays are depicted.

FIGS. 9A-9G shows tumor-derived stromal cells that trigger enhanced proliferation in macrophage with features of self-renewal. FIG. 9A shows the expression of Ly6A in a subset of F4/80^{High} macrophages. Flow cytometry for Ly6A (Sca-1) and F4/80 in BMDMs co-cultured with oTME cells for 10 days or left unperturbed as control cells (n=5 replicates). Data are shown as mean±SD., 2-tailed unpaired Student's t-test. FIG. 9B shows flow cytometry plots quantifying expression of Ly6A (Sca-1) in F4/80^{High} macrophages (left) and Ki67+ macrophages (right) of mammary tumors (n=4 replicates). Data are shown as mean±SD., 2-tailed unpaired Student's t-test. FIG. 9C shows immunofluorescence for F4/80, Fibronectin (FN1) and endogenous tdTomato (mT) in Rosa26mTmG BMDMs co-cultures with oTME cells for 10 days and stained as indicated (n=3 replicates). Scale bars, 100 μm. Fluorescent signal ratios of F4/80 over tdTomato were plotted as a function of their association with FN1+ cells (n=282 cells). Data are shown as mean±SD. Mann-Whitney test. FIG. 9D shows flow cytometry of Ki67 in BMDMs co-cultured either with oTME, tumor epithelial, or stromal-like cells or left alone as control cells (+M-CSF 10 ng/mL). Data quantification of Ki67 and F4/80 are presented at far right (n=3 replicates). Data are shown as mean±SD., one-way ANOVA test, Bonferroni-corrected. FIG. 9E shows a cytokine array analysis of secretomes from sorted tumor epithelial, stromal-like or unsorted parental oTME cells. Conditioned media were collected and probed for 111 cytokines, growth factors and interleukins. FIG. 9F shows EdU incorporation in macrophages co-cultured with tumor epithelial or stromal-like cells (cell contact) or following treatment with their conditioned medium (CM) for 7 days. Individual dots represent fields of view from 3 independent experiments. Data (Tumor Epithelial: CM n=9; cell-contact n=34; Stroma-like: CM n=10; cell-contact n=34) are shown as mean±SD., 2-way ANOVA test, Bonferroni-corrected. FIG. 9G shows EdU/BrdU dual pulse-chase labelling in BMDMs co-cultured with oTME cells for 7 days, labeled with EdU for 72 hrs, and then pulsed with BrdU for another lhr. Macrophages were analyzed by flow cytometry for EdU and BrdU and quantified for single and double positivity (n=3 replicates). Data are shown as mean±SD., 2-way ANOVA test, Bonferroni-corrected.

FIG. 10A-10C shows that mammary gland fibroblasts promote macrophage proliferation following activation by tumor cells. FIG. 10A shows an experimental design. mTmG macrophages and WT fibroblasts from mammary gland no. 4 (MG4) were FACS-sorted (n=6 mice and 2 replicates) and plated either with or without tumor epithelial cells. Fluorescent images were taken on day-1 and day-10. FIG. 10B shows flow cytometry analysis of macrophage proliferation from cell cultures in A, using CD45, F4/80, CD11b, and Ki67 antibodies. FIG. 10C shows Secretome analysis of supernatants from A using cytokine arrays. Note the enhanced macrophage proliferation in the presence of cytokines of activated fibroblasts (POSTN and IL-6).

FIGS. 11A-11D show macrophage proliferation begins at early stages of mammary gland transformation and synchronized with tumor cell proliferation. FIG. 11A shows IHC staining of Ki67 in sequential stages in mammary gland transformation starting from normal, hyperplasia, and late carcinoma of MMTV-PyMT tumor model. Scale bars, 100 μm. FIG. 11B shows immunofluorescence staining of mammary tissues from normal (left) and MMTV-PyMT hyperplasia lesions (right) for IBA1 and Ki67. Intraepithelial ductal macrophages (inset A) and stromal macrophages (inset B). Individual dots represent fields of view from 4 mammary tumors. Scale bars, 100 μm. Data (fields of view; Healthy n=12, Hyperplasia n=92) are shown as mean±SD., Mann—Whitney U test. FIG. 11C shows immunofluorescence staining of MMTV-PyMT late carcinoma lesions for IBA1 and Ki67. Dashed lines highlight cycling (A inset; Ki67+) and non-cycling (B inset; Ki67^{neg}) tumor areas. Dots represent fields of view from 6 tumors. Scale bars, 100 μm. Data (fields of view; Non-cycling n=19, Cycling n=35) are shown as mean±SD., Mann-Whitney test. FIG. 11D shows CD163 and Ki67 staining in human breast tumors. Inset A highlights proliferative areas (Ki67^{high}) while inset B marks low-proliferating areas (Ki67^{Low}). Dots represent fields of view from 5 tumors. Scale bars, 100 μm. Data (fields of view; Non-cycling n=33, Cycling n=24; In tumor n=39, In stroma n=88) are shown as mean±SD., Mann-Whitney U test.

FIG. 12A-12E shows in vivo characterization of stroma-associated macrophages. FIG. 12A shows growth kinetics of tumors induced by tumor epithelial cells alone, or by tumor epithelial cells (EpCAM+) enriched with PDGFRA+CD24^{neg} cells (40:60 ratio; n=4 replicates). Tumor volumes were recorded and compared at endpoint. Data are shown as mean±SD., Mann-Whitney test. FIG. 12B shows IHC staining for Vimentin (stroma) and IBA1 (macrophages) in tumor transplants from A at study endpoint. Scale bars, 500 μm. FIG. 12C shows immunoprofiling of the changes in the myeloid landscape in mammary tumors enriched with PDGFRA+CD24^{neg} stromal-like cells. After exclusion of Ly6G+ granulocytes, three populations were classified: (i) Ly6C+MHC-II N egF4/80^{low} (M-MDSC), (ii) MHC-II⁺F4/80^{High}CD11b^{High} (SAMs) and (iii) MHC-II⁺F4/80^{Int}CD11b^{Low} (TEMs). Frequencies were calculated as the percentage of CD45+ cells. Data are shown as mean±SD. (n=4 replicates), 2-way ANOVA test, Bonferroni-corrected. FIG. 12D shows immunofluorescence analysis of IBA1 (red), F4/80 (green) and Ki67 (white) in mammary tumors. Dashed lines mark tumor nests. F4/80^{High} SAMs appear yellow while F4/80^{Int} TEMs appear reddish. Fluorescent signal ratios of F4/80 over IBA1 were recorded and plotted as a function of their spatial localization. Dots represent fields of view (n=24) from 4 tumors. Data are shown as mean±SD. two-tailed paired non-parametric Wilcoxon test. Scale bar, 50 μm. FIG. 12E shows flow cytometry quantification of Ki67+ macrophages in mammary tumors. As deduced from Ki67 staining in D, Ki67+ macrophages were significantly enriched in F4/80^{High} macrophages which associate with SAMs (n=5 replicates). Data are shown as mean±SD., two-tailed paired non-parametric Wilcoxon test.

FIG. 13A-13B shows gating strategy for myeloid cells in mammary PyMT tumors. FIG. 13A shows representative gating strategy for myeloid cells in mammary tumors. Myeloid cells were selected based on (1) CD45+ and CD11b+, followed by (2) exclusion of Ly6G+ granulocytes (3) and classified into two subpopulations based on MHC-II expression: (4) MHC-II^{Neg}Ly6C+F4/80^{Low} (M-MDSC), and MHC-II+ macrophages that were further divided based on CD11b expression (5): Cd11b^{Low}F4/80^{int} (TEMs) and Cd11b^{High}F4/80^{High} (SAMs). Cell proportions were quantified (n=7 replicates). Data are shown as mean±SD., 2-way ANOVA test, Bonferroni-corrected. FIG. 13B shows immunofluorescence staining of mammary tumor macrophages using IBA1 (red), F4/80 (green), and Ki67 (white). Dashed lines mark the tumor borders (Ki67+ cells), and rectangles indicate magnified areas. Peri-tumor stromal macrophages (F4/80^{High}) appear green while intratumoral macrophages (F4/80^{Low}) appear red. Signal profile lines of F4/80 and IBA1 from the magnified area were plotted below.

FIGS. 14A-14H show monocyte differentiation on tumor epithelial or stromal cells dictates phenotypic plasticity in macrophages. FIG. 14A shows purified BM monocytes from mTmG mice (n=4 replicates) were differentiated into macrophages for 7 days in the presence of either EpCAM+tumor epithelial cells or PDGFRA+CD24^{neg} stromal-like cells. FIG. 14B shows representative images of monocyte-derived macrophage morphology following 7 days of differentiation with EpCAM+ tumor epithelial cells or PDGFRA+CD24^{neg} stromal-like cells. Scale bars, 50 μm. FIG. 14C shows macrophage morphology in human breast cancer as a function of their spatial localization. Left panel shows tumor nests with CD163+ dendritic macrophages while the right panel shows granular CD163+ macrophages in tumor stroma. Scale bars, 100 μm. FIG. 14D shows flow cytometry of CD206, LGALS3, IL-1b and CD11a in monocyte-derived macrophages following 7 days of differentiation with EpCAM+ tumor epithelial cells or PDGFRA+CD24^{neg} stromal-like cells (n=4 replicates). Data are shown as mean±SD, 2-way ANOVA test, Bonferroni-corrected. FIG. 14E shows scavenging activity of SAMs and TEMs. Monocyte-derived macrophages were generated as in FIG. 14D and purified splenic EGFP+ NK cells (methods) were added for additional 5 days. Flow analysis was performed for TIM-4 (scavenging) and images were taken. Data (SAM n=3; TEM n=4) are shown as mean±SD., student t-test. FIG. 14F shows flow cytometry analysis of CD206 and PD-L1 in M-MDSCs, SAMs and TEMs from mammary tumors (n=6 replicates). Data are shown as mean±SD, 2-way ANOVA test, Bonferroni-corrected. FIG. 14G shows tumor tissues from (FIG. 14F) were stained for CD206, PD-L1, and IBA1 using IHC. Left panel highlights CD206+ macrophages in peritumoral stroma (FIG. 14A) and intratumoral stroma (FIG. 14B). Right panel shows PD-L1+ staining only in SAMs, confirming flow data in (FIG. 14G). FIG. 14H shows Immunofluorescence staining for CD206 and CD163 in a cohort of breast tissues. Dashed lines highlight the normal or malignant epithelial areas and show CD206 expression only on SAMs (n=4 replicates). Dots represent fields of view from areas of normal stroma (n=16), normal epithelium (n=15), tumor stroma (n=15), tumor epithelium (n=13) of as mean±SD., 2-way ANOVA test, Bonferroni-corrected.

FIGS. 15A-15B shows immunoprofiling of monocyte-derived SAMs and TEMs. FIG. 15A shows concatenated visualization of flow cytometry values of FSC-A (size) and SSC-A (granularity) in monocyte-derived macrophages from mTmG mice (n=4 replicates) that differentiated in the presence of EpCAM+ tumor epithelial or PDGFRA+CD24^{neg} stromal-like cells. Data are shown as mean±SD. Student t-test. FIG. 15B shows immunoprofiling of monocyte-derived macrophages co-cultured for seven days with the indicated cell types or left alone treated with M-CSF as control. Cell proportions were quantified (n=3 replicates). Data are shown as mean±SD., 2-way ANOVA test, Bonferroni-corrected.

FIG. 16A-16B shows CD206 and CD11c expression in adipose and ductal macrophages of the human mammary gland. FIG. 16A shows immunofluorescence staining for CD206 and CD163 in human normal mammary glands. The numbers of double-positive cells were quantified from 3 tissue samples. Dots represent fields of view (n=24), shown as mean±SD. FIG. 16B shows immunofluorescence staining for CD11c and CD163 in normal, DCIS, and invasive human breast cancer. The adjacent section was stained with H&E (n=4 replicates). Dashed lines highlight healthy glands (upper panel) or non-invasive malignant epithelial areas (middle; DCIS), showing that CD11c is predominantly expressed on CD163+ TEMs. In invasive tumor areas (bottom), CD11c is expressed on all CD163+ macrophages. Data (fields of view; n=25 each) are shown as mean±SD., Mann-Whitney test.

FIGS. 17A-17F shows Notch4 targeting reduces macrophage proliferation in mammary tumors. FIG. 17A shows a Gene Set Enrichment Analysis of Gene Ontology showing the significant enrichment of Notch signaling pathway (GO:0007219; FDR=0.00065; left, enrichment scores; right, rank positions of member genes). FIG. 17B shows immunoblotting of cleaved Notch intracellular domain (NICD), ARG1, SMAD2/3 (TGF-β), MAPK, and PI3K confirming active Notch signaling only in educated BMDMs and not in unperturbed control cells (treated with M-CSF). FIG. 17C shows the impact of γ-secretase inhibition on proliferation of M2-educated macrophages. BMDMs were co-cultured with oTME cells for 7 days and treated either with CompE (101xM) or DMSO as a control for additional 10 days. Cells were analyzed by flow cytometry for Ki67, F4/80, and CD11b. Data (n=3) are shown as mean±SD., 2-way ANOVA test, Bonferroni-corrected. FIG. 17D is a pre-clinical study design. FIG. 17E shows established mammary tumor transplants in mTmG mice (n=8 replicates) were treated with NOTCH4 neutralizing antibodies (15 m/kg body weight, dosed every 3 days, intraperitoneally) or vehicle (PBS) as a control for 20 days. Tumor sizes were recorded by caliper and shown as mean±SD., 2-way ANOVA test, Bonferroni-corrected. At the trial endpoint, lungs were collected and stained with H&E (right). FIG. 17F shows flow cytometry of macrophage proliferation in mammary tumors following NOTCH4 neutralization (n=6 replicates). At study endpoint, tumor transplants from (FIG. 17E) were scored for Ki67 in macrophages and in tumor cells (PyMT; mT^{neg} CD45^{neg}). Data are shown as mean±SD., 2-way ANOVA test, Bonferroni-corrected. Macrophage abundance quantified as percent of CD45+ cells. Data are shown as mean±SD., Mann-Whitney test.

FIG. 18 shows Protease activity of Adam17 is required for macrophage proliferation. EdU incorporation in BMDMs pre-treated with Adam17 protease inhibitor (A17Pro) or PBS as control and plated with oTME cells for seven days. Cells were then labeled with EdU for 48 hrs and analyzed by flow cytometry (n=4 replicates). Data are shown as mean±SD., Mann-Whitney test.

FIGS. 19A-19B show Notch4 neutralization in mammary tumors does not affect macrophage abundance and angiogenesis. FIG. 19A shows Flow cytometric data showing NOTCH4 neutralization effect on the abundance of M-MDSCs, TEM and SAMs, and frequencies of CD206+ macrophages in control and Notch4-treated mammary tumors (n=6 replicates). Data are shown as mean±SD., 2-way ANOVA test, Bonferroni-corrected. FIG. 19B shows immunofluorescence staining of CD31, IBA1, and Ki67 in the indicated tumor transplants from A. Scale bars, 100 μm.

FIGS. 20A-20D show tdTomato expression in tissues from MMTV-Cre LSL-tdTomato mice. FIG. 20A shows flow cytometry analysis for tdTomato in indicated tissues from MMTV-Cre LSL-tdTomato mice. Similar tissues from MMTV-Cre mice were used as control. FIG. 20B are graphs of qPCR analysis of E-cadherin (Cdh1), Vimentin (Vim), and Pymt transgene expression, normalized to CD24int mesenchymal cells (n=4 replicates). Data are shown as mean±SE. FIG. 20C shows tumor growth kinetics of EpCAM+ or EpCAM− orthotopic transplants in the mammary gland of C57BL/6 WT mice (n=5 replicates). Lung and liver were collected, and metastases were visualized by H&E staining. FIG. 20D contains violin plots of annotated single-cell transcriptomes for extracellular matrix and lineage marker genes in the basal and CD24neg stromal-like cells, and Gene Set Enrichment Analysis (GSEA) of extracellular matrix pathway.

FIG. 21 shows tumor-induced immunosuppressive effect of macrophages on NK and T cells. Splenic EGFP NK cells (from mTmG Flt3-Cre mice) were FACS-sorted and plated with oTME for 72hsr in the presence or absence of macrophages (tdTomato). The number of NK cells per field of view was quantified (n=3).

FIG. 22 shows growth inhibition of CD4 and CD8 T cells by oTME macrophages. Splenic isolated T-cells were labeled with CFSE, stimulated with CD3/CD28 activating antibodies for 1 hr, and plated as indicated. Cell proliferation and lineages of T-cells were analyzed by flow cytometry after 72hrs (n=4).

FIG. 23 shows a stable isotope labeling by amino acids in cell culture (SILAC)-based strategy for analysis of intracellular and secrets proteins in oTME macrophages. Intracellular proteins, left: Macrophages are differentiated with M-CSF separately in growth medium supplemented with either light or heavy amino acid (AA) for seven days. Then heavy AA-labeled macrophages are added to heavy AA labeled oTME culture for another seven days, FACS-purified, and mixed 1:1 with light AA-labeled macrophages for MS analysis. Secreted proteins, right: Macrophages are differentiated separately in heavy AA, then added to light AA-labeled oTME culture, conditioned media are collected and analyzed by MS. Heavy peptides are macrophage-secreted proteins.

### DETAILED DESCRIPTION

A first aspect of the present disclosure is directed to a method of inhibiting an immunosuppressive phenotype in a population of macrophages. This method involves administering to the population of macrophages, an agent selected from a cyclin-dependent kinase 4 (Cdk4) inhibitor, a tumor necrosis factor related apoptosis-inducing ligand receptor 2 (TRAIL-R2) inhibitor, a protein tyrosine kinase 2 beta (Ptk2b) inhibitor, Notch-4 inhibitor and combinations thereof under conditions effective to inhibit the immunosuppressive phenotype in the population of macrophages.

In accordance with this aspect of the disclosure, the population of macrophages comprises macrophages having an M2 phenotype. Macrophages exhibiting a type-2 (M2) phenotype are often characterized as being anti-inflammatory and immunosuppressive as they suppress T-cell responses and are involved in the Th2-type immune response. The type-2 macrophage phenotype facilitates tissue repair, wound healing, and is profibrotic. Type-2 macrophages often undesirably infiltrate and surround tumors, where they provide an immunosuppressive microenvironment that promotes rather than suppresses tumor progression. Type-2 macrophages are characterized by high surface expression of I1-4R, FecR, Dectin-1, CD136, CD206, and CD209A. Type-2 macrophages include IL-4/IL-13-stimulated macrophages, IL-10-induced macrophages, and immune complex-triggered macrophages. In some embodiments, the administering is carried out to a population of macrophages having an M2 phenotype in vitro. In some embodiments, the administering is carried out to a population of macrophages having an M2 phenotype in vivo.

Administering the Cdk4 inhibitor, the TRAIL-R2 inhibitor, the Ptk2b inhibitor, or combination thereof to the population of macrophages comprising an M2 phenotype induces a change in the macrophage phenotype. In some embodiments, the administering will induce an

M1 phenotype. Macrophages exhibiting a type-1 phenotype are pro-inflammatory, and are capable of either direct (pathogen pattern recognition receptors) or indirect (Fc receptors, complement receptors) recognition of pathogens and tumor antigens (i.e., they exhibit anti-tumor activity). Type-1 macrophages produce reactive oxygen species and secrete pro-inflammatory cytokines and chemokines, such as, for example, but without limitation, TNFα, IL-1, IL-6, IL-IL-18, IL-23, and iNOS. Thus, the conversion of type-2 macrophages to type-1 macrophages in accordance with the methods described herein can be monitored or assessed by assessing the levels of the aforementioned cytokines and chemokines. Type-1 macrophages can also be characterized by their expression of high levels of MHC, costimulatory molecules, and FCγR. The type-1 phenotype is triggered by GM-CSF and further stimulated by interferon-γ (IFN-γ), bacterial lipopolysaccharide (LPS), or tumor necrosis factor a (TNFα), and is mediated by several signal transduction pathways involving signal transducer and activator of transcription (STAT), nuclear factor kappa-light-chain-enhancer of activated B cells (NFKB), and mitogen-activated protein kinases (MAPK). These events enhance the production of agents such as the reactive oxygen species and nitric oxide (NO) and promote subsequent inflammatory immune responses by increasing antigen presentation capacity and inducing the Thl immunity through the production of cytokines such as IL-12.

In accordance with this aspect of the disclosure, suitable Cdk4 inhibitors for use in the method of inhibiting an immunosuppressive phenotype in a population of macrophages include, without limitation, palbociclib (6-acetyl-8-cyclopentyl-5-methyl-2-[(5-piperazin-1-ylpyridin-2-yl)amino]pyrido[2,3-d]pyrimidin-7-one), ribociclib (7-cyclopentyl-N,N-dimethyl-2-[(5-piperazin-1-ylpyridin-2-yl)amino]pyrrolo[2,3-d]pyrimidine-6-carboxamide), abemaciclib (N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine), voruciclib (2-[2-chloro-4-(trifluoromethyl)phenyl]-5,7-dihydroxy-8-[(2R,3S)-2-(hydroxymethyl)-1-methylpyrrolidin-3-yl]chromen-4-one), and trilaciclib (4-[[5-(4-methylpiperazin-1-yl)pyridin-2-yl]amino]spiro[1,3,5,11-tetrazatricyclo[7.4.0.02,7]trideca-2,4,6,8-tetraene-13,1′-cyclohexane]-10-one).

In accordance with this aspect of the disclosure, suitable Ptk2B inhibitors for use in the method of inhibiting an immunosuppressive phenotype in a population of macrophages include, without limitation, PF-00562271 (N-methyl-N-[3-[[[2-[(2-oxo-1,3-dihydroindo1-5-yl)amino]-5-(trifluoromethyl)-4-pyrimidinyl]amino]methyl]-2-pyridinyl]methanesulfonamide is a member of indoles), conteltinib (2-[[2-[2-methoxy-4-[4-(4-methylpiperazin-1-yl)piperidin-1-yl]anilino]-6,7-dihydro-5H-pyrrolo[2,3-d]pyrimidin-4-yl]amino]-N-propan-2-ylbenzenesulfonamide), and NVP-TAE226 (2-[[5-chloro-2-(2-methoxy-4-morpholin-4-ylanilino)pyrimidin-4-yl]amino]-N-methylbenzamide).

In accordance with this aspect of the disclosure, the TRAIL-R2 inhibitor for use in the method of inhibiting an immunosuppressive phenotype in a population of macrophages is a monoclonal antibody inhibitor. In one embodiment, the TRAIL-R2 inhibitor is TRAIL-R2 or Tnfrsfl2a receptor (TWEAK) monoclonal antibody. Other genes and proteins involved in enhancing macrophage immunosuppressive

phenotype in the tumor environment are identified in Table 1. Accordingly, the method of inhibiting immunosuppressive phenotype in a population of macrophages can also involve administering an inhibitor of one or more of the genes and it encoded protein identified in Table 1. Known inhibitors of these modulators are also provided in Table 1. In any embodiment, one or more inhibitors identified in Tablel are used alone or in combination with each other or in combination with a Cdk4 inhibitor, a TRAIL-R2 inhibitor, or a Ptk2b inhibitor as described supra to inhibit the immunosuppressive phenotype in a population of macrophages. Such inhibition can be carried out in vitro or in vivo.

The method of inhibiting an immunosuppressive phenotype in a population of macrophages can be carried out in vivo to treat a variety of conditions in a subject where the immunosuppressive phenotype of macrophage plays a causative role in the progression of the condition or contributes to one or more symptoms of the condition. For example, the method of inhibiting an immunosuppressive phenotype in a population of macrophages can comprise administering Cdk4 inhibitor, the TRAIL-R2 inhibitor, the Ptk2b inhibitor, or combination thereof to a subject in need thereof Subjects who would benefit from inhibiting the immunosuppressive phenotype of macrophage using a Cdk4 inhibitor, the TRAIL-R2 inhibitor, the Ptk2b inhibitor, or combination thereof as described herein include those suffering endometriosis (see e.g., Hogg et al., “Endometriosis-associated Macrophages: Origin, Phenotype, and Function,” Front. Endocrinol. 11:7 (2020), which is hereby incorporated by reference in its entirety), systemic sclerosis (see e.g., Bhandari et al., “Profibrotic Activation of Human Macrophages in Systemic Sclerosis,” Arthritis & Rheumatology 72(7): 1160-69 (2020), which is hereby incorporated by reference in its entirety), and idiopathic pulmonary fibrosis (see e.g., Morse et al., “Proliferating SPP1/MERTK-expressing Macrophages in Idiopathic Pulmonary Fibrosis,” Eur. Respir J. 54(2): 1802441 (2019), which is hereby incorporated by reference in its entirety).

In any embodiment, the method is carried out in vivo to a subject having a tumor. In accordance with this method, the Cdk4 inhibitor, the TRAIL-R2 inhibitor, the Ptk2b inhibitor, or combination thereof is administered to macrophages within the tumor microenvironment to induce an immunomodulatory response to the tumor. Subjects that are suitable for such administration, are those subject having a cold tumor. A “cold tumor” is a tumor that contains few if any infiltrating T cells. Exemplary cold tumors that can be treated in accordance with this and other methods described herein include, without limitation, breast tumors, pancreatic tumors, ovarian tumors, prostate tumors, colon tumors, solid tumors, gliomas, myelomas, liver tumors, and kidney tumors.

The status of a subject's tumor (i.e., a hot tumor vs. a cold tumor) is typically determined by immunological parameters, in particular by assessing lymphocyte infiltration and IFN-γ status. This can be determined by employing known immunohistochemical methods to a core needle biopsy. Tumors with low lymphocyte infiltration, and commonly high infiltration of immunosuppressive myeloid cells, such as M2 macrophages are considered “cold tumors” and suitable for treatment in accordance with the methods described herein. M2 macrophage accumulation can be determined using immunohistological methods suitable for the detection of markers, including, but not limited to, CD163, CD68, CD206, or the combination of CD163 and PD-L1.

Administering the Cdk4 inhibitor, the TRAIL-R2 inhibitor, the Ptk2b inhibitor, or a combination thereof will induce an immunomodulatory response or immunomodulatory phenotype in the macrophages surrounding the tumor.

In some embodiments, the Cdk4 inhibitor, TRAIL-R2 inhibitor, or Ptk2b inhibitor is administered to the subject as a part of a combination therapy or therapeutic. In some embodiments, the combination therapeutic comprises the Cdk4 inhibitor, the TRAIL-R2 inhibitor, and/or the Ptk2b inhibitor in combination with a checkpoint inhibitor. In some embodiments, the combination therapeutic comprises the Cdk4 inhibitor, the TRAIL-R2 inhibitor, and/or the Ptk2b inhibitor in combination with a pro-inflammatory agent.

As used herein, the term “combination therapy” or “combination therapeutic” refers to the administration of two or more therapeutic agents, e.g., an agent that inhibits Cdk4, an agent that inhibits TRAIL-R2, and an agent that inhibits Ptk2b, a checkpoint inhibitor, a pro-inflammatory agent, and combinations thereof. In some embodiments, the combination therapy is co-administered in a substantially simultaneous manner, such as in a single capsule or other delivery vehicle having a fixed ratio of active ingredients. In some embodiment, the combination therapy is administered in multiple capsules or delivery vehicles, each containing an active ingredient. In some embodiments, the therapeutic agents of the combination therapy are administered in a sequential manner, either at approximately the same time or at different times. In all of the embodiments, the combination therapy provides beneficial effects of the drug combination in treating cancer, particularly in treatment-resistant cancers as described herein.

In some embodiments, the agents of the combination therapeutic are administered concurrently. In other embodiments, the agent that inhibits Cdk4, the agent that inhibits TRAIL-R2, and/or the agent that inhibits Ptk2b is administered prior to administering the checkpoint inhibitor and/or the proinflammatory agent.

In accordance with this and all aspects of the disclosure, suitable checkpoint inhibitors include, without limitation, a programmed death-ligand 1 (PD-L1) inhibitor, a programmed cell death protein 1 (PD-1) inhibitor, a cytotoxic T-lymphocyte-associated protein 4 (CTLA-4) inhibitor, and combinations thereof.

In some embodiments, the checkpoint inhibitor is a PD-1 inhibitor. Suitable PD-1 inhibitors include, without limitation, the monoclonal antibodies of Pembrolizumab (Merck), Nivolumab (Britsol Myers Squibb), Pidilizumab (Medivation), and Cemiplimab (Regeneron).

In some embodiments, the checkpoint inhibitor is a PD-L1 inhibitor. Suitable PD-L1 inhibitors include, without limitation, the monoclonal antibodies of Atezolizumab (Genentech), Avelumab (Pfizer), Durvalumab (AstraZeneca).

In some embodiments, the checkpoint inhibitor is a CTLA-4 inhibitor. A suitable CTLA-4 inhibitor is the monoclonal antibody, Ipilimumab (Bristol Myers Squibb).

In some embodiments, the subject having a cold tumor is administered a pro-inflammatory agent in combination with the Cdk4 inhibitor, the TRAIL-R2 inhibitor, and/or the Ptk2b inhibitor. In accordance with this embodiment, suitable pro-inflammatory agents, includes, without limitation, GM-CSF, an OX40 (CD134, TNFSRSF4) activation antibody, and a TREM2 (Triggering Receptor Expressed on Myeloid Cells) blocking antibody or inhibitory peptide.

Granulocyte-macrophage colony stimulating factor (GM-CSF) is an immunostimulatory monomeric glycoprotein secreted by macrophages, T cells, mast cells, and natural killer cells. Pharmaceutical analogs of GM-CSF suitable for use in the methods described herein include sargramostim and molgramostim.

OX40 is a co-stimulatory molecule expressed by activated immune cells. Agonist antibodies suitable for administration in accordance with the methods of the present disclosure include, without limitation, INCAGN01949 IgG (Gonzalez et al., “INCAGN01949: A Novel Anti-OX40 Agonist Antibody with the Potential to Enhance Tumor Specific T-cell Responsiveness, While Selectively Depleting Intratumoral Regulatory T Cells,” Cancer Res. 76(14 Suppl) 3204 (2016), which is hereby incorporated by reference in its entirety) and PF-4518600 (El-Khoueiry et al., “Analysis of OX40 Agonist Antibody (PF-4518600) in Patients with Hepatocellular Carcinoma,” J. Clin. Oncol. 38(4): (2020), which is hereby incorporated by reference in its entirety).

Another aspect of the present disclosure relates to a method of inhibiting macrophage proliferation in a population of cells comprising macrophages. This method involves administering a Notch-4 inhibitor or a TYK2 inhibitor to the population of cells under conditions effective to inhibit macrophage proliferation in said population of cells.

The method of inhibiting macrophage proliferation can be carried out in vivo to treat a variety of conditions in a subject where macrophage proliferation plays a causative role or in some way contributes to one or more symptoms of the condition. For example, the method of inhibiting macrophage proliferation in a population of cells can comprise administering the Notch-4 inhibitor or TYK2 inhibitor to a subject in need thereof Subjects who would benefit from inhibition of macrophage proliferation using a Notch-4 inhibitor or TYK2 inhibitor as described herein include those suffering from inflammatory conditions, such as asthma, atherosclerosis, arthritis (e.g., rheumatoid arthritis, osteoarthritis); metabolic diseases, such as diabetes and obesity related adipose inflammation (see e.g., Ponzoni et al., “Targeting Macrophages as a Potential Therapeutic Interventions Impact on Inflammatory Diseases and Cancer, Int. J. Mol. Sci. 19(7): 1953 (2018), which is hereby incorporated by reference in its entirety); autoimmune diseases, such as systemic lupus erythematosus, systemic sclerosis, primary biliary cholangitis, Sjögren's syndrome, and inflammatory bowel disease (see e.g., Ma et a., “The Role of Monocytes and Macrophages in Autoimmune Diseases: A Comprehensive Review,” Front. Immunol. 10:1140 (2019), which is hereby incorporated by reference in its entirety); chronic inflammation and age-related chronic inflammatory disease (see e.g., Oishi and Manabe, “Macrophages in Age-related Chronic Inflammatory Diseases,” Nature: Aging and Mechanisms of Disease 2:16018 (2016), which is hereby incorporated by reference in its entirety); and hyperinflammatory conditions associated with ‘cytokine storm’, such as infections (viral infections), sepsis, systemic juvenile idiopathic arthritis, adult onset Still disease, connective tissue diseases, cancer and cancer immunotherapy (see e.g., McGonagle et al., “Immune Cartography of Macrophage Activation Syndrome in the COVID-19 Era,” Nature Reviews Rheumatology 17:145-157 (2021), which is hereby incorporated by reference in its entirety).

Another condition that would benefit from a decrease in macrophage proliferation is cancer. Accordingly, another aspect of the present disclosure relates to a method of treating a tumor in a subject. This method involves administering, to a subject having a tumor, a Notch-4 inhibitor or a TYK2 inhibitor, wherein said administering induces an anti-tumor immune response in the subject.

In accordance with this aspect of the present disclosure, the subject having a tumor may have a tumor selected from the group consisting of a breast tumor, pancreatic tumor, ovarian tumor, prostate tumor, lung tumor, colon tumor, solid tumor, glioma, melanoma, myeloma, liver tumor, and kidney tumor. In some embodiments, the subject has a cold tumor as described above. In some embodiments, the tumor is characterized by tumor cells overexpressing Notch-4.

Suitable Notch-4 inhibitors include, without limitation protein or peptide Notch-4 inhibitors, e.g., anti-Notch-4 antibody-based molecules; nucleic acid molecule inhibitors, e.g., a Notch-4 antisense oligonucleotide inhibitor; and small molecule inhibitors of Notch-4.

In any embodiment, a suitable Notch-4 inhibitor for use in inhibiting macrophage proliferation or inducing an anti-tumor immune response in a subject having a tumor, is an anti-Notch-4 antibody-based molecule, including, for example, a Notch-4 antibody, Notch-4 binding fragment thereof, or a Notch-4 antibody derivative.

Human Notch-4 has the amino acid sequence the of SEQ ID NO: 1 (UniProt Accession No. Q99466) as provided below.

MQPPSLLLLLLLLLLLCVSVVRPRGLLCGSFPEPCANGGTCLSLSLGQGTCQCAPGFLGE
TCQFPDPCQNAQLCQNGGSCQALLPAPLGLPSSPSPLTPSFLCTCLPGFTGERCQAKLED
PCPPSFCSKRGRCHIQASGRPQCSCMPGWTGEQCQLRDFCSANPCVNGGVCLATYPQIQC
HCPPGFEGHACERDVNECFQDPGPCPKGTSCHNTLGSFQCLCPVGQEGPRCELRAGPCPP
RGCSNGGTCQLMPEKDSTFHLCLCPPGFIGPDCEVNPDNCVSHQCQNGGTCQDGLDTYTC
LCPETWTGWDCSEDVDECETQGPPHCRNGGTCQNSAGSFHCVCVSGWGGTSCEENLDDCI
AATCAPGSTCIDRVGSFSCLCPPGRTGLLCHLEDMCLSQPCHGDAQCSTNPLTGSTLCLC
QPGYSGPTCHQDLDECLMAQQGPSPCEHGGSCLNTPGSFNCLCPPGYTGSRCEADHNECL
SQPCHPGSTCLDLLATFHCLCPPGLEGQLCEVETNECASAPCLNHADCHDLLNGFQCICL
PGFSGTRCEEDIDECRSSPCANGGQCQDQPGAFHCKCLPGFEGPRCQTEVDECLSDPCPV
GASCLDLPGAFFCLCPSGFTGQLCEVPLCAPNLCQPKQICKDQKDKANCLCPDGSPGCAP
PEDNCTCHHGHCQRSSCVCDVGWTGPECEAELGGCISAPCAHGGTCYPQPSGYNCTCPTG
YTGPTCSEEMTACHSGPCLNGGSCNPSPGGYYCTCPPSHTGPQCQTSTDYCVSAPCENGG
TCVNRPGTFSCLCAMGFQGPRCEGKLRPSCADSPCRNRATCQDSPQGPRCLCPTGYTGGS
CQTLMDLCAQKPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPSGYLCQCAPGY
DGQNCSKELDACQSQPCHNHGTCTPKPGGFHCACPPGFVGLRCEGDVDECLDQPCHPTGT
AACHSLANAFYCQCLPGHTGQWCEVEIDPCHSQPCFHGGTCEATAGSPLGFICHCPKGFE
GPTCSHRAPSCGFHHCHHGGLCLPSPKPGFPPRCACLSGYGGPDCLTPPAPKGCGPPSPC
LYNGSCSETTGLGGPGFRCSCPHSSPGPRCQKPGAKGCEGRSGDGACDAGCSGPGGNWDG
GDCSLGVPDPWKGCPSHSRCWLLFRDGQCHPQCDSEECLEDGYDCETPPACTPAYDQYCH
DHFHNGHCEKGCNTAECGWDGGDCRPEDGDPEWGPSLALLVVLSPPALDQQLFALARVLS
LTLRVGLWVRKDRDGRDMVYPYPGARAEEKLGGTRDPTYQERAAPQTQPLGKETDSLSAG
FVVVMGVDLSRCGPDHPASRCPWDPGLLLRFLAAMAAVGALEPLLPGPLLAVHPHAGTAP
PANQLPWPVLCSPVAGVILLALGALLVLQLIRRRRREHGALWLPPGFTRRPRTQSAPHRR
RPPLGEDSIGLKALKPKAEVDEDGVVMCSGPEEGEEVGQAEETGPPSTCQLWSLSGGCGA
LPQAAMLTPPQESEMEAPDLDTRGPDGVTPLMSAVCCGEVQSGTFQGAWLGCPEPWEPLL
DGGACPQAHTVGTGETPLHLAARFSRPTAARRLLEAGANPNQPDRAGRTPLHAAVAADAR
EVCQLLLRSRQTAVDARTEDGTTPLMLAARLAVEDLVEELIAAQADVGARDKWGKTALHW
AAAVNNARAARSLLQAGADKDAQDNREQTPLFLAAREGAVEVAQLLLGLGAARELRDQAG
LAPADVAHQRNHWDLLTLLEGAGPPEARHKATPGREAGPFPRARTVSVSVPPHGGGALPR
CRTLSAGAGPRGGGACLQARTWSVDLAARGGGAYSHCRSLSGVGAGGGPTPRGRRFSAGM
RGPRPNPAIMRGRYGVAAGRGGRVSTDDWPCDWVALGACGSASNIPIPPPCLTPSPERGS
PQLDCGPPALQEMPINQGGEGKK

Suitable Notch-4 antibody-based molecules for use in the methods disclosed herein bind to one or more epitopes in the Notch-4 amino acid sequence. Suitable Notch-4 antibody-based molecules include those known in the art. In some embodiments, the Notch-4 antibody or antibody-based molecule is the Notch-4 antibody or a derivative thereof disclosed in U.S. Pat. No. 9,527,921 to Sakamoto et al., which is hereby incorporated by reference in its entirety, having a heavy chain CDR1 (H-CDR1) sequence of SYGMS (SEQ ID NO: 2); a H-CDR2 sequence of GFTESSYGMS (SEQ ID NO: 3) or a HCDR-2 sequence of TINSNGGRTYYPDSVKG (SEQ ID NO: 4), or a HCDR-2 sequence of TINSNGGRTY (SEQ ID NO: 5); and a HCDR-3 sequence of DQGFAY (SEQ ID NO: 6). In some embodiments, the Notch-4 antibody comprises a light chain CDR 1 (LCDR-1) sequence of KASQDVGTAVA (SEQ ID NO: 7); a LCDR-2 sequence of WASTRHT (SEQ ID NO: 8); and a LCDR-3 sequence of QQYSSYPWT (SEQ ID NO: 9).

In some any embodiment, the Notch-4 antibody-based molecule for use in the methods disclosed herein has a heavy chain variable region amino acid sequence of SEQ ID NO: 10 as provided below or a humanized version thereof as disclosed in U.S. Pat. No. 9,527,921 to Sakamoto et al., which is hereby incorporated by reference in its entirety.

(SEQ ID NO: 10)
EVQLVESGGGLVQPGGSLKLSCAASGFTFSSYGMSWVRQTPDKRLELVA
TINSNGGRTYYPDSVKGRFTISRDNAKNTLYLQMSSLKSEDTAMYYCAR
DQGFAYWGQGTLVTVSA.

In any embodiment, the Notch-4 antibody-based molecule for use in the methods disclosed herein has a heavy chain variable region amino acid sequence that is at least 80%, 85%, 90%, 91%,92%, 93%, 94%, 95%, 96%, 97%, 98% or 99% identical to the amino acid sequence of SEQ ID NO: 10. Suitable variant heavy chain variable region amino acid sequences are disclosed in U.S. Pat. No. 9,527,921 to Sakamoto et al., which is hereby incorporated by reference in its entirety.

In some embodiments, the Notch-4 antibody-based molecule for use in the methods disclosed herein has a light chain variable region amino acid sequence of SEQ ID NO: 11 as provided below or a humanized version thereof as disclosed in U.S. Pat. No. 9,527,921 to Sakamoto et al., which is hereby incorporated by reference in its entirety.

(SEQ ID NO: 11)
DIVMTQSHKFMSTSVGDRVSITCKASQDVGTAVAWDIVMTQSHKFMSTS
VGDRVSITCKASQDVGTAVAWFTLTISNVQSEDLADYFCQQYSSYPWTF
GGGTKLEIK.

In any embodiment, the Notch-4 antibody-based molecule for use in the methods disclosed herein has a light chain variable region amino acid sequence that is at least 80%, 85%, 90%, 91%,92%, 93%, 94%, 95%, 96%, 97%, 98% or 99% identical to the amino acid sequence of SEQ ID NO: 11. Suitable variant light chain variable region amino acid sequences are disclosed in U.S. Pat. No. 9,527,921 to Sakamoto et al., which is hereby incorporated by reference in its entirety. Notch-4 antibody-based molecules suitable for use in the methods described

herein include, without limitation full antibodies, epitope binding fragments of whole antibodies, and antibody derivatives. An epitope binding fragment of an antibody can be obtained through the actual fragmenting of a parental antibody (for example, a Fab or (Fab)2 fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

In any embodiment, the Notch-4 antibody-based molecule suitable for use in the methods described herein is an intact immunoglobulin or a molecule having a Notch-4 epitope-binding fragment thereof As used herein, the terms “fragment”, “region”, and “domain” are generally intended to be synonymous, unless the context of their use indicates otherwise. Naturally occurring antibodies typically comprise a tetramer, which is usually composed of at least two heavy (H) chains and at least two light (L) chains. Each heavy chain is comprised of a heavy chain variable (VH) region and a heavy chain constant (CH) region, usually comprised of three domains (CH1, CH2 and CH3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (VL) region and a light chain constant (C L) region. Light chains include kappa chains and lambda chains. The heavy and light chain variable regions are responsible for antigen recognition, while the heavy and light chain constant regions may mediate the binding of the immunoglobulin to host tissues or factors, including various cells of the immune system (e.g., effector cells) and the first component (Clq) of the classical complement system. The V_{H} and V_{L} regions can be further subdivided into regions of hypervariability, termed “complementarity determining regions,” or “CDRs,” that are interspersed with regions of more conserved sequence, termed “framework regions” (FR). Suitable Notch-4 heavy chain and light chain CDRs are described supra. Each V_{H} and V_{L} region is composed of three CDR domains and four FR domains arranged from amino-terminus to carboxy-terminus in the following order: FR1-CDR1-FR2-CDR2-FR3-CDR3-FR4. Suitable Notch-4 V_{H} and V_{L} regions are described supra. The variable regions of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally-occurring antibody in amino acid sequence.

Fragments of antibodies (including Fab and (Fab)₂ fragments) that exhibit Notch-4 epitope-binding ability are also suitable for use in the methods described herein. Notch-4 epitope binding fragments can be obtained, for example, by protease cleavage of intact antibodies. Single domain antibody fragments possess only one variable domain (e.g., V_{L} or V_{H}). Examples of the epitope-binding fragments encompassed within the present invention include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain, (v) dAb fragments (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a V_{H} or V_{L} domain and also called domain antibodies (Holt et al. “Domain Antibodies: Proteins For Therapy,” Trends Biotechnol. 21(11):484-490 (2003), which is hereby incorporated by reference in its entirety); (vi) nanobodies (Revets et al. “Nanobodies As Novel Agents For Cancer Therapy,” Expert Opin. Biol. Ther. 5(1):111-124 (2005), which is hereby incorporated by reference in its entirety), and (vii) isolated complementarity determining regions (CDR). A suitable Notch-4 epitope-binding fragment for use in the methods described herein may contain 1, 2, 3, 4, 5 or all 6 of the CDR domains of the Notch-4 antibody described supra (i.e., SEQ ID NOs: 2-9).

Such antibody fragments may be obtained using conventional techniques known to those of skill in the art. For example, F(ab′)₂ fragments may be generated by treating a full-length antibody with pepsin. The resulting F(ab′)₂ fragment may be treated to reduce disulfide bridges to produce Fab′ fragments. Fab fragments may be obtained by treating an IgG antibody with papain and Fab′ fragments may be obtained with pepsin digestion of IgG antibody. A Fab′ fragment may be obtained by treating an F(ab′)₂ fragment with a reducing agent, such as dithiothreitol. Antibody fragments may also be generated by expression of nucleic acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). Nucleic acid molecules encoding heavy chain and light chain regions of the Notch-4 antibodies described supra are disclosed in U.S. Pat. No. 9,527,921 To Sakamoto et al., which is hereby incorporated by reference in its entirety. For example, a chimeric gene encoding a portion of a F(ab′)₂ fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Notch-4 antibody derivatives suitable for use in the methods described herein include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region, which may be encoded by separate genes. Such gene sequences or their encoding cDNA are joined, using recombinant methods, by a flexible linker (typically of about 10, 12, 15 or more amino acid residues) that enables them to be made as a single protein chain in which the VL and VH regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., not less than about 9 residues) to enable the V_{L} and V_{H} regions of a different single polypeptide chains to associate together, one can form a bispecific antibody, having binding specificity for two different epitopes.

In another embodiment, the antibody derivative suitable for use in the methods described herein is a divalent or bivalent Notch-4 single-chain variable fragment, engineered by linking two scFvs together either in tandem (i.e., tandem scFv), or such that they dimerize to form diabodies (Holliger et al. “‘Diabodies’: Small Bivalent And Bispecific Antibody Fragments,” pu Proc. Natl. Acad. Sci. (U.S.A.) 90(14), 6444-8 (1993), which is hereby incorporated by reference in its entirety). In yet another embodiment, the antibody is a trivalent single chain variable fragment, engineered by linking three scFvs together, either in tandem or in a trimer formation to form triabodies. In another embodiment, the antibody is a tetrabody of four single chain variable fragments. In another embodiment, the antibody is a “linear antibody” which is an antibody comprising a pair of tandem Fd segments (VH-CH1-VH-CH1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3).

Antibody fragments and derivatives suitable for use in the methods described herein also include antibody-like polypeptides, such as chimeric antibodies and humanized antibodies, and antibody fragments retaining the ability to specifically bind to the Notch-4 (epitope-binding fragments) provided by any known technique, such as enzymatic cleavage, peptide synthesis, and recombinant techniques.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Either of the human light chain constant regions, kappa or lambda, may be used. If desired, the class of a Notch-4 antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In one embodiment, the Notch-4 antibody-based molecules suitable for use in the methods described herein are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A. F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. When non-human antibodies are prepared with respect to a particular antigen, the variable regions can be “reshaped” or “humanized” by grafting CDRs derived from non-human antibody onto the FRs present in the human antibody to be modified. Suitable methods for humanizing non-human antibodies, including those, described herein are known in the art see e.g., Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M.S. et al., “Chimeric And Humanized Antibodies With Specificity For The CD33 Antigen,” J. Immunol. 148:1149-1154 (1992), which are hereby incorporated by reference in their entirety. In some embodiments, humanized Notch-4 antibodies of suitable for use in the methods described herein preserve all CDR sequences (for example, a humanized antibody containing all six CDRs from the mouse antibody). In other embodiments, humanized Notch-4 antibodies suitable for use in the methods described herein have one or more CDRs (one, two, three, four, five, six) which are altered with respect to the original antibody. Suitable humanized Notch-4 antibodies for use in the methods disclosed herein are disclosed in U.S. Pat. No. 9,527,921 To Sakamoto et al., which is hereby incorporated by reference in its entirety.

In another embodiment, the Notch-4 inhibitor is a small molecule Notch-4 inhibitor. Suitable Notch-4 inhibitors include, without limitation, RO4929097 (RG-4733) having the following structure.

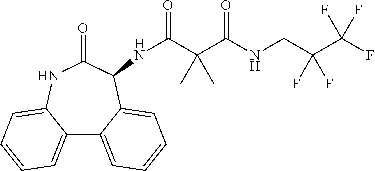 [SMILES: CC(C)(C(=O)NCC(F)(F)C(F)(F)F)C(=O)N[C@@H]1C(=O)Nc2ccccc2-c2ccccc21]

Another small molecule Notch-4 inhibitor that can be utilized in accordance with the methods of the present disclosure, i.e., to inhibit macrophage proliferation and self-renewal in the tumor microenvironment, is Nirogacestat (PF-030840140) having the following structure,

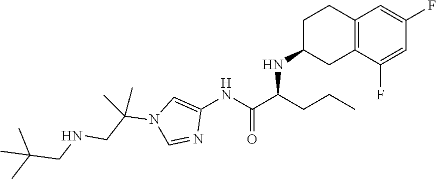 [SMILES: CCC[C@H](N[C@H]1CCc2cc(F)cc(F)c2C1)C(=O)Nc1cn(C(C)(C)CNCC(C)(C)C)cn1]

In another embodiment, the method of inhibiting macrophage proliferation in a population of cells comprising macrophages involves administering a TYK2 inhibitor to the population of cells under conditions effective to inhibit macrophage proliferation in said population of cells. Suitable TYK2 inhibitors for use in accordance with this method of the disclosure include, without limitation, PF-06826647 (3-(cyanomethyl)-3-[4-[6-(1-methylpyrazol-4-yl)pyrazolo[1,5-a]pyrazin-4-yl]pyrazol-1-yl]cyclobutane-1-carbonitrile), NDI-031407 (Gracey et al., J. Clin. Invest. 130(4): 1863-78 (2020), which is hereby incorporated by reference in its entirety), Deucravacitinib (BMS-986165; 6-(cyclopropanecarbonylamino)-4-[2-methoxy-3-(1-methyl-1,2,4-triazol-3-yl)anilino]-N-(trideuteriomethyl)pyridazine-3-carboxamide), and others known in the art.

In some embodiments, the method of treating a tumor in a subject as disclosed herein further comprises administering to the selected subject a checkpoint inhibitor in combination with the Notch-4 inhibitor and/or TYK2 inhibitor. Suitable checkpoint inhibitors are described supra, and include, without limitation a PD-L1 inhibitor, a PD-1 inhibitor, a CTLA-4 inhibitor, and combinations thereof

In some embodiments, the method of treating a tumor in a subject as disclosed herein further comprises administering to the selected subject a pro-inflammatory agent in combination with the Notch-4 inhibitor. Suitable pro-inflammatory agent include those described supra, including without limitation, GM-CSF, an OX40 activation antibody, and a TREM2 blocking antibody.

In accordance with this method and all methods described herein that involve administration of one or more therapeutic agents as described herein to a subject having a tumor, said administration of the agent(s) alone or in combination is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of cancer to be treated.

A therapeutically effective amount of the therapeutic agent(s) alone or in combination in the methods disclosed herein is an amount that, when administered over a particular time interval, increases the subject's immune response to the tumor, which further leads to a slowing or halting of cancer growth, cancer regression, cessation of symptoms, etc. The therapeutic agents for use in the presently disclosed methods may be administered to a subject one time or multiple times. In those embodiments where the therapeutic agents are administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer, e.g., presence of an immune response, or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the therapeutic agents administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

The effectiveness of the methods of the present application in increasing the immune response or decreasing immune-tolerance can be assessed, for example, by assessing changes in cancer burden and/or disease progression following treatment with the therapeutic agents as described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, cancer burden and/or disease progression is evaluated using imaging techniques including, e.g., X-ray, computed tomography (CT) scan, magnetic resonance imaging, multiparametric ultrasound (mpUS), multiparametric magnetic resonance imaging (mpMRI), and nuclear imaging (positron emission tomography [PET]) (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Cancer regression or progression may be monitored prior to, during, and/or following treatment with one or more of the therapeutic agents described herein.

In some embodiments, the effectiveness of the methods described herein may be evaluated, for example, by assessing immunological parameters, such as lymphocyte infiltration and IFN-y status. In some embodiments, the methods described are suitable for increasing the subject's immune response to the tumor, are effective to inhibit disease progression, inhibit cancer growth/spread, relieve cancer-related symptoms, inhibit tumor-secreted factors (e.g., tumor-secreted hormones), delay the appearance of primary or secondary cancer tumors, slow development of primary or secondary cancer tumors, decrease the occurrence of primary or secondary cancer tumors, slow or decrease the severity of secondary effects of disease, arrest tumor growth, and/or achieve regression of cancer in a selected subject.

In some embodiments, the methods described herein reduce the rate of cancer growth in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In certain embodiments, the methods described herein reduce the rate of cancer invasiveness in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In specific embodiments, the methods described herein reduce the rate of cancer progression in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In various embodiments, the methods described herein reduce the rate of cancer recurrence in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In some embodiments, the methods described herein reduce the rate of metastasis in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more.

Another aspect of the present disclosure relates to a combination therapeutic. In some embodiments, the combination therapeutic comprises a Notch-4 inhibitor and a checkpoint inhibitor. Suitable Notch-4 antibodies and checkpoint inhibitors of the combination therapeutic are described supra.

In some embodiments, the combination therapeutic a Notch-4 inhibitor and a pro-inflammatory agent. Suitable Notch-4 antibodies and pro-inflammatory agents of the combination therapeutic are described supra.

Another aspect of the present disclosure is directed to an organotypic tumor microenvironment model (TME) culture system. The TME culture system comprises an isolated population of cells, said population comprising tumor epithelial cells, mesenchymal stromal cells, fibroblasts. In one embodiment, the fibroblasts are immortalized fibroblasts.

The immortalization of fibroblasts can be induced or arise spontaneously as a result of the cultures system. To induce fibroblast immortalization, the fibroblasts can be transformed with one or more reagents to facilitate immortalization. Suitable methods of immortalizing cells are well known in the art and suitable for use in accordance with this embodiment. For example, the cells can be transformed with a viral gene (e.g., large T-antigen of the simian virus (SV-40), HPV E6 or E7 genes) to promote viral gene overexpression which in turn suppresses expression of the endogenous cell cycle modulators (e.g., retinoblastoma and p53 genes) to induce uncontrolled proliferation. Alternatively, fibroblasts can be immortalized by inducing expression of genes that confer immortality, e.g., telomerase (hTERT). hTERT expression prevents normal telomere shortening to abate the senescence process and enables the cell to undergo infinite cell division. In another embodiment, the fibroblasts become immortalized as a result of being in culture with tumor epithelial cells. In either embodiment, the fibroblasts of the TME cell culture system described herein are different from fibroblasts which exist naturally in the tumor environment because they exhibit an immortalized phenotype.

In some embodiments, the TME culture system further comprises one or more additional cell types. Suitable cell types include, without limitation, macrophages, endothelial cells, T cell, NK cells, and dendritic cells. The TME cell culture system, which replicates the tumor environment, is useful for examining and delineating cell-to-cell interactions in the tumor environment as well as test and screen candidate agents and compounds for manipulating these interactions.

In one embodiment, the TME culture system comprises macrophages having an M1 phenotype. In any embodiment, these macrophages of the model have an expression profile of Ki67NegCD11chiArg1Neg indicating a non-proliferative, pro-inflammatory phenotype. In another embodiment, the TME culture system comprises macrophages having an M2 phenotype. In any embodiment, these macrophages of the model have an expression profile of

Ki67+ CD11clowArgl+indicating a proliferative, immunosuppressive phenotype.

In some embodiments, the TME culture system comprises a population of tumor epithelial cells and mesenchymal cells derived from a breast tumor as described herein. In accordance with this embodiment, the tumor epithelial cells are characterized by EpCAM³⁰/CD49f^{high}/CD24^{high}/CD61⁻ expression.

In any embodiment, the population of tumor epithelial cells, mesenchymal stromal and any of the other one or more cell types present in the cell culture are derived from a tumor or tumor environment. In any embodiment, the cells are derived from the tumor or tumor environment associated with a breast tumor, pancreatic tumor, ovarian tumor, prostate tumor, lung tumor, colon tumor, solid tumor, glioma, melanoma, myeloma, liver tumor, and kidney tumor.

In any embodiment, the cells of the TME culture system are primary cells, isolated directly from a tumor or the surrounding tumor environment. In some embodiments, the population of cells in the TME cultures is a syngeneic population of cells. In some embodiments, the population of cells is a population of human cells. In some embodiments, the population of cells is a population of rodent cells, such as murine or rat cells.

In any embodiment, one or more cell populations of the TME culture are modified to express a detectable label. For example, in any embodiment, cells of the model can be modified to express a detectable label, such as fluorescent protein, where the expression of the detectable label is controlled by a cell specific promoter or an inducible promoter system (e.g., cre-recombinase), and useful to identify or track the presence of a particular cell type (e.g., allow the tracking of cell differentiation). In other embodiments, expression of the detectable label can be coupled to the expression of any protein of interest. Such modification may involve transient transfection or stable transductions of the cells of the culture with an expression vector comprising a nucleic acid molecule encoding the detectable label.

The in vitro TME culture model described herein is cultured or maintained using standard tissue culture procedures. Appropriate growth and culture conditions for various mammalian cell types are well known in the art. The cells in the in vitro TME culture model may be seeded onto and/or within a substrate from a suspension so that they are evenly distributed at a relatively high surface and/or volume density. The cell suspensions may comprise approximately about 1×10⁴ to about 5×10⁷ cells/ml of culture medium, or approximately about 2×10⁶ cells/ml to about 2×10⁷ cells/ml, or approximately about 5×10⁶ cells/ml. The optimal concentration and absolute number of cells will vary with cell type, growth rate of the cells, substrate material, and a variety of other parameters. The suspension may be formed in any physiologically acceptable medium, preferably one that does not damage the cells or impair their ability to adhere to the substrate. Appropriate mediums include standard cell growth media (e.g., DMEM with 10% FBS).

Cells of the in vitro TME culture model are cultured in a media that generally includes essential nutrients and, optionally, additional elements such as growth factors, salts, minerals, vitamins, etc., that may be selected according to the cell type(s) being cultured. A standard growth media includes Dulbecco's Modified Eagle Medium, low glucose (DMEM), with 110 mg/L pyruvate and glutamine, supplemented with 10-20% fetal bovine serum (FBS) or calf serum and 100 U/ml penicillin. The culture media may also contain particular growth factors selected to enhance cell survival, differentiation, secretion of specific proteins, etc. In some embodiments, the culture of the TME culture model is performed in a sterile environment under standard culture conditions, e.g., incubation at 37° C. in an incubator containing a humidified atmosphere of 95% air and 5% CO₂.

As described herein, the TME culture system can be utilized to identify targetable mediators of the tumor microenvironment for the development of innate immune cell targeted immunotherapies. In some embodiments, the step of identifying targetable mediators of the tumor microenvironment can further be used to test the efficacy of a therapeutic agent or combination of therapeutic agents. Such assessment comprises performing at least one test or multiple tests to evaluate (qualitatively or quantitatively) a modification of the morphology and/or composition of the organotypic culture at the tissue level (for example at the level of the vessels), at the cellular level and/or at the molecular level (protein, DNA, RNA, etc.) by immunohistochemistry or western Blot, by flow cytometry, by a microscopy technique or by protein analysis. Depending on the results obtained, an anti-cancer therapy is identified. In some embodiments, the method can be adapted to identify a personalized anti-cancer therapeutic regimen by developing a TME culture system utilizing TME cells of a cancer patient. Since the organotypic TME culture model obtained by the methods described herein is more representative of the in vivo situation than a single cell line or a primary cell culture, it is an excellent model for the screening of therapeutic compounds.

Thus, in another aspect, the present disclosure relates to the use of an organotypic TME culture system described herein for the screening compounds which may have immunotherapeutic properties against the tumor. In one embodiment, the disclosure encompasses a method of screening for compounds capable of having immunotherapeutic properties against cancer where the method involves the steps of incubating an organotypic TME culture as described herein in the presence of a test compound, and observing the effects of the test compound on the organotypic culture. In any embodiment, the TME culture is representative of tumor environment selected from a lung tumor, esophagus tumor, stomach tumor, liver tumor, pancreas tumor, colon tumor, breast tumor, ovary tumor, cervix tumor, prostate tumor, testes tumor, skin tumor, thyroid tumor, adrenal gland tumor, etc. As noted supra, the TME culture comprises tumor epithelial cells, immortalized fibroblasts, and stromal cells of the tumor of interest. Further cell types of interest (e.g., macrophages, NK cells, T cells, etc.) are added to the model to examine cell specific contribution to the tumor environment and identify druggable targets of immunotherapeutics.

Accordingly, another aspect of the present disclosure is directed to a method of identifying candidate compounds or agents capable of modulating the tumor microenvironment. In one aspect, the method involves identifying a candidate compounds or agent capable of modulating macrophage activity in a tumor environment. This method involves providing the organotypic tumor microenvironment model (TME) culture system as described herein, wherein said system comprises macrophages, in addition to the fibroblasts, tumor epithelial cells, and stromal cells. The system can optionally comprise one or more other cell types. The method further involves administering the candidate compound to the culture system and assessing one or more markers of macrophage activity in the culture systems before and after said administering. A candidate compound capable of modulating macrophage activity in the tumor environment is identified based on said assessing.

As described herein, the TME culture system was utilized to identify compounds that modulate macrophage phenotype in the tumor environment. Macrophages in the tumor environment often exhibit a type-2 phenotype, which is characterized as being anti-inflammatory and immunosuppressive as they suppress T-cell responses and are involved in the Th2-type immune response. Type-2 macrophages are characterized by high surface expression of Il-4R, FeϵR, Dectin-1, CD136, CD206, and CD209A. The induction of Arg1 is considered as one of the bona fide hallmarks of M2-like macrophages and associated with anti-inflammatory and tissue repair phenotypes. Therefore, as described herein, Arg1 induction is a useful surrogate for TME-education. Type-2 macrophages include IL-4/IL-13-stimulated macrophages, IL-10-induced macrophages, and immune complex-triggered macrophages. Thus, a candidate agent capable of modulating macrophage immunosuppressive tumor phenotype is one that can decrease the expression of the aforementioned proteins and/or promote the expression of markers of Type-1 macrophages. Gene involved in modulating macrophage tumor phenotype and candidate agents identified using this screen are provided in Table 1 below.

In another aspect, the method involves identifying a candidate compound capable of modulating NK cell activity in a tumor environment. This method comprises providing the organotypic TME culture system, wherein said system further comprises NK cells, in addition to the fibroblasts, tumor epithelial cells, and stromal cells. The system can optionally comprise one or more other cell types. The method further involves administering the candidate compound to the culture system and assessing one or more markers of NK cell activity in the culture systems before and after said administering. A candidate compound capable of modulating NK cell activity in the tumor environment is identified based on said assessing. For example, in one embodiment, this method is used for identifying a candidate compound capable of modulating NK cell exhaustion in a tumor environment. NK cell exhaustion is marked by a decrease in the expression of several cytokines (e.g., IFN-γ) and cytolytic molecules (e.g., granzymes, perforin, FasL and TRAIL). Thus, the screening method described herein is useful for identifying genes/proteins involved in modulating NK cell exhaustion as well as drug candidates for reversing such exhaustion.

In another aspect, the method involves identifying a candidate compound capable of modulating T cell activity in a tumor environment. This method comprises providing the organotypic TME culture system, wherein said system further comprises T cells, in addition to the fibroblasts, tumor epithelial cells, and stromal cells. The system can optionally comprise one or more other cell types. The method further involves administering the candidate compound to the culture system and assessing one or more markers of T cell activity in the culture systems before and after said administering. A candidate compound capable of modulating T cell activity in the tumor environment is identified based on said assessing. For example, in one embodiment, this method is utilized for identifying a candidate compound capable of modulating T cell exhaustion in a tumor environment. T cell exhaustion is marked by a decrease in the expression of several surface markers (e.g., CD127 and CD62L) and effector molecule activity (e.g., decrease in IL-2, TNF-α, IFN-γ, and GranB), as well as an increase in inhibitory receptor expression (e.g., PD-1, Tim-3, and Lag-3). Thus, the screening method described herein is useful for identifying genes/proteins involved in modulating T cell exhaustion as well as drug candidates for reversing such exhaustion.

In another aspect, the method involves identifying genetic changes in one or more cell types of the tumor environment. This method comprises providing the organotypic TME culture system, wherein said system may further comprise one or more of macrophages, NK cells, T cells, dendritic cells, and/or endothelial cells in addition to the fibroblasts, tumor epithelial cells, and stromal cells. The method further involves subjecting the cells of the TME culture system to genetic analysis after time in culture. Genetic analysis involves employment of any art known method of analyzing gene expression in the system (e.g., qPCR, RNA-sequence analysis, CRISPR/Cas9 genomic screen, etc.) as well as analysis of genetic changes in any one or more of the cell types (i.e., detection of nucleotide deletions, substitutions, and/or additions that lead to mutations in the resulting protein). This analysis is useful for identifying target genes/proteins of the tumor environment contributing to the tumorigenic process. Further, administering a candidate compound to the culture system and assessing changes in the expression or function of one or more identified genetic elements in the culture system can be used to identify new tumor therapeutics.

### EXAMPLES

The following examples are provided to illustrate embodiments of the present disclosure but are by no means intended to limit its scope

### Materials and Methods of Examples 1-8

Pooled CRISPR sgRNA Libraries Construction: CRISPR single-guide RNAs (sgRNAs) were optimized for on-target activity with minimal off-targets in the human genome using a similar approach as done previously (Meier et al., “GUIDES: sgRNA Design for Loss-of-Function Screens,” Nat. Methods 14(9):831-832 (2017), which is hereby incorporated by reference in its entirety). CRISPR sgRNAs were targeted within —200 nt upstream of the gene's transcription start site (TSS). Four guides per gene were designed including 1000 non-targeting sgRNAs as control. Custom ssDNA oligos were synthesized by Twist Bioscience and cloned into the lentiCRISPRV2 plasmid (Sanjana et al., “Improved Vectors and Genome-Wide Libraries for CRISPR Screening,” Nat. Methods 11:783-784 (2014), which is hereby incorporated by reference in its entirety) using a method similar to the one described in (Shalem et al., “Genome-Scale CRISPR-Cas9 Knockout Screening in Human Cells,” Science 343:84-87 (2014), which is hereby incorporated by reference in its entirety). Individual sgRNAs were cloned as previously described (Parnas et al., “A Genome-Wide CRISPR Screen in Primary Immune Cells to Dissect Regulatory Networks,” Cell 162:675-686 (2015), which is hereby incorporated by reference in its entirety).

Library Lentivirus Production: HEK-293T cells were plated (8×10⁶) in T225 flasks and transfected the day after with a DNA mix consist of: 25 μg lentiCRISPRv2 sgRNA construct, 14 μg pMD2.G, and 20μg psPAX2 in 2.5 mL Opti-MEM+PEI (polyethylenimine) at the ratio of 3 μL per 1 μg DNA mix. Day after transfection, the growth medium was refreshed and cells were incubated for another 48 hrs to allow accumulation of viruses. Supernatants were collected, cell debris were removed by centrifugation at 1000 g for 10 min at 4 C. Virus-containing supernatants were tested for virus titer, aliquoted and stored in −80 C.

Pooled Whole Genome CRISPR Screen of Arg1-EYFP Macrophages: Bone marrow-derived macrophages (BMDMs) (3.2×10⁸) were generated from Arg1-EYFP mice (n=45), seeded at 50% confluency on RetroNectin-coated (50μg/mL) 10cm plates, and infected with pooled lentiviral library at an MOI of 1. The inventors aimed for 500 cells per guide in order to achieve a 500× library representation after —20% infection efficacy puromycin selection. Transduced BMDMs were incubated for 48hrs with puromycin (1 μg/mL) and M-CSF (long/mL). Puromycin-resistant BMDMs were pooled together and a batch equivalent to 1.0× representations of sgRNA libraries (500× per gene) was snapped-frozen as “library representation”, while rest of BMDMs were co-cultured with oTME cells for 10 days to initiate M2-education. Educated Arg1-EYFP BMDMs were collected, immunostained for CD45, CD11b, F4/80, and FACS-sorted into two groups according to EYFP-Arg1: (i) the top 10% of EYFP+ (M2) and (ii) EYFP^{neg} BMDMs (“M2-resistant”). To detect the sgRNAs and gene identities, genomic DNA (gDNA) was purified using Qiagen DNeasy Blood & Tissue Kit according to the manufacturer's instruction. The integrated sgRNA cassettes were amplified by PCR (PCR1), and Illumina sequencing adapters were attached by nested PCR (PCR2) as previously described (Shalem et al., “Genome-Scale CRISPR-Cas9 Knockout Screening in Human Cells,” Science 343:84-87 (2014), which is hereby incorporated by reference in its entirety). The resulting gDNA amplicon library was sequenced at ˜50-75 reads per guide on an Illumina MiSeq 150v3 kit.

Chromium 10× Single-Cell RNA-Seq: BMDMs were co-cultured with oTME cells for 2 (early) and 10-days (late), or left alone with M-CSF for same time intervals. Cells were collected, clumps were removed through a 40 μm filter, and cells (n=4000 per condition) were loaded on Chromium platform to generate cDNA following the manufacturer's protocol. Single-cell cDNA libraries were prepared using the Chromium Single Cell 3′ Library & Gel Bead Kits v2 (PN-120237, PN-120236, PN-120262) according to the manufacturer's instructions. Samples were sequenced at an average of 50,000 reads per cell.

RNA Isolation for qRT—PCR: RNA from cells was extracted using TRIzol™ Reagent (Thermo Fisher Scientific; 15596026) and 1-5ug RNA were used to prepare cDNA using RNA to cDNA EcoDryT™ kit (Clontech; 639549). RNA from FACS-sorted cells were extracted using TRIzol™ LS Reagent (Thermo Fisher Scientific; 10296010) and processed according to manufacturer's protocol. For qPCR reactions, mouse Taqman probes (Applied

Biosystems) were used for quantifying expression of Cdh1 (Mm01247357_m1), Csf1 Mm00432686_m1, Csf2 (Mm01290062_m1), Csf3 Mm00438334_m1, Vim (Mm01333430_m1). Expression values were normalized to both Hprt (Mm03024075_m1) and Gapdh (Mm99999915_g1) as housekeeping genes.

Mouse Work and Strains: Animal procedures were approved by the Institutional Animal Care and Use Committee (IACUC) of the Research Animal Resource Center (RARC) at Weill Cornell Medicine (Protocol: 2016-0058). MMTV-PyMT (Stock No: 022974), Arg1-EYFP (Stock No: 015857), and wild-type C57BL/6J (Stock No: 000664) were purchased from The Jackson Laboratory (USA). Rosa26^{mTmG} (Stock No: 007676, mTmG) mice were a gift from Dr. Johhan Joyce (MSKCC).

Tumor Engraftment and Processing: Females of the MMTV-PyMT model developed spontaneous tumors after 100-120 days post birth. To generate orthotopic tumor transplants, tumor cells (1.0×10⁶) were injected in 50 μL of 50% Matrigel (Corning) in serum-free DMEM, into the 4th mammary gland of 7-10 weeks of wild-type (WT) C57BL/6 female mice. Prior to injection, mice were anesthetized with 2% isoflurane and cells were injected under the nipple into the fat pad. Tumor dimensions (width and length) were measured using a digital caliper and tumor volumes were calculated as V=(L×W²)/2.

Neutralization of NOTCH4 in vivo: To target NOTCH4 in mammary tumors, wild-type C57BL/6 female mice were engrafted with tumor epithelial cells in the 4th mammary glands and allowed to establish palpable tumors. Then, mice were pooled and randomized into two arms: vehicle-treated (PBS) or Notch4-treated with anti-Notch4 monoclonal antibodies (BioXcell, clone HMN4-14). Mice were dosed every 3 days intraperitoneally with 15 μg/kg body weight.

Mouse Bone Marrow Derived Macrophages (BMDMs): Mouse BMDMs were obtained from femurs and tibias of 6-8-week-old B6 mice. Bone marrow cells were flushed onto a 40 μM strainer using a 25G×⅝ needle washed with RPMI. Bone marrow cells were gently meshed through the 40 μm strainer using a 1 ml plunger. After centrifugation at 300 g 4° C., for 5 minutes, cells were resuspended in DMEM medium, 10% FBS and 1% Pen-strep supplemented with 10 ng/mL recombinant murine M-CSF. Growth medium was replenished every other day with fresh 10 ng/mL M-CSF. On day-7 >95% of the cells were CD11b+ Ly6C/Ly6G− (Gr1−) F4/80+ macrophages.

Mouse Bone Marrow Monocytes Isolation: Mouse bone marrow was obtained from femurs and tibias as previously described with differentiation of BMDMs. Bone marrow cells were resuspended with 5 mL of RBC Lysis Buffer (ThermoFisher Scientific) for five minutes incubation on ice to remove red blood cells, washed with serum-free RPMI, and monocytes are purified using negative selection monocytes isolation kit (130-100-629; Miltenyi).

Chemicals and Biological Reagents: 99LN-parental cells were generated as previously described (Quail et al., “Obesity Alters the Lung Myeloid Cell Landscape to Enhance Breast Cancer Metastasis Through IL5 and GM-CSF,” Nat. Cell Biol. 19:974-987 (2017), which is hereby incorporated by reference in its entirety). Isolations of tumor epithelial or stromal cells were performed by FACS-sort using EpCAM and PDGFRA antibodies. Cells were maintained in DMEM-GlutaMAX (Gibco; 10566016) supplemented with 10% FBS (Gibco), and 1% Pen-strep (ThermoFisher Scientific). Cells were routinely verified to be mycoplasma-free using commercial kit (Lonza). M-CSF, IL-4, and IL-13 recombinant proteins were from Peprotech.

EdU Incorporation: Cells were incubated with 10μM EdU (5-ethynyl-2′-deoxyuridine) without changing conditioned growth medium for the required duration and analyzed by flow cytometry according to the manufacturer's instructions (A10202; Thermo Fisher Scientific). For EdU imaging, cells were plated on coverslips, incubated with EdU (10 μM), fixed with 4% PFA, and immunostained with desired antibodies prior to EdU staining protocol (C10337).

Immune Cell Isolation for Flow Cytometry from Mammary Gland or Mammary Tumors: MMTV-PyMT tumors were collected from euthanized female mice, washed in cold PBS and digested by mechanical dissociation, using gentleMACSTM Dissociator (Miltenyi Biotec) and mouse tumor dissociation kit (Miltenyi Biotec) according the manufacturer's instructions. To remove cell clumps and undigested tissues, cell suspensions were passed through 70 μm and then 40 μm filters, and even cell numbers were analyzed by flow cytometry. To isolate cells from murine mammary glands, the 4th and 5th glands were collected and digested with 4 mg/ml collagenase type 4 (porcine origin, Sigma), 4 μg/mlDNase I (Sigma) at 37° C. with periodic vortexing. Cells were further mashed through 100 μm filters, and then passed through 40 μm. Cells were collected, counted, and analyzed by flow cytometry.

Whole Mount Imaging: For whole mount immunofluorescence imaging, approximately 3 mm³ pieces of inguinal fat-pad from 12 weeks old PyMT-MMTV females were incubated in 4% paraformaldehyde (PFA) (Electron Microscopy) diluted in PBS for 30 min at room temperature with agitation, permeabilized with PBS Triton 0.3% (Sigma T8787) 4% BSA for 1 hour at room temperature. Samples were stained with directly conjugated antibodies in PBS Triton for 1 hour. Samples were rinsed with PBS 3X and mounted on cavity slides (Sigma) with Fluoromount G (eBioscience). The antibodies used are the following: Anti-mouse F4/80 eFluor570 (eBioscience, clone BM8 (1/200), anti-mouse EpCAM Alexa Fluor 488 (Biolegend clone G8.8, 1/100), Anti-mouse CD140a Alexa Fluor 647 (eBioscience: APAS; PDGFR-a, 1/100). Z-stacks of 30 μm to 60 μm with 0.8 μm consecutive intervals and tile scans were acquired using LSM880 Zeiss microscope with 40×/1.3 objective(oil).

3D Primary Mammary Gland Culture and Time-Lapse Imaging: After sacrifice, inguinal fat pads were dissected from 12 week old PyMT-MMTV Rosa26mTmG females.

Tissues were minced into small pieces and digested in PBS containing Collagenase II (sigma, ref: C6885-1g), prepared at 0.8 mg/mL in PBS+0.5% BSA+CaCl₂ 5 mmol/L for 20 min at 37° C. Once processed, all samples were filtered through 100 mm strainers and approximately 2×10⁵ total cells were resuspended in 1001iL drop of Growth Factor Reduced Matrigel on ice, incubated 30 min at 37° C. and cultivated in DMEM F12 10%FBS with Alexa Fluor 647 anti-mouse F4/80 (Biolegend 123122, 1/200) in Ibidi 24 wells microplate. Imaging was performed on day 3 of culture using LSM-880 in imaging chambers maintained at 37° C. 5% CO₂, 20% O₂ and 90% relative humidity. Seven consecutives stacks of 3 mm intervals were captured every 5 min per position using 20× objective for >12 hours. At endpoint, cell cultures were fixed for 10 min in 4% PFA after imaging, stained as indicated, and imaged as for whole mount imaging.

Flow Cytometry and Fluorescence-Activated Cell Sorting (FACS): Flow cytometry data were collected using BD LSRFortessa, BD FACSCanto II, and BD FACSAria III was used for FACS-sort. FlowJo X was used for data analysis and generation of flow plots for figures. For analyzing live cells from tissues by flow cytometry, mice were anaesthetized with ketamine/xylazine cocktail and perfused with 30 mL cold PBS using cardiac puncture. Cells from dissociated tissues were filtered through 70 μm then 40 μm filters to generate a single-cell suspension. Cells (1-2×10⁶) were then incubated with 2× Fc Block solution (1:50, CD16/32 BD Biosciences) in FACS-buffer (2% FBS-PBS, 2 mM EDTA for 20 min in 8° C.). Conjugated antibodies were added to cells and incubated for 30 min in ° C. in the presence of the FC-blocking solution. Stained cells were washed twice and resuspended with a FACS-buffer containing DAPI (1 m/mL) to exclude dead/compromised cells. Mammary gland eosinophils were defined as CD45⁺CD11b⁺F4/80^{low} SiglecF⁺ and excluded together with DAPI⁺ dead cells by staining with BV421-SiglecF.

CFSE T-Cell Proliferation Assay: T-cell proliferation was measured using CFSE assay (ThermoFisher Scientific). CD3+ T-cells were negatively isolated (EasySep Mouse T Cell Isolation Kit) from spleen of WT mouse, labeled with 5μM CSFE and stimulated with CD3e (1:100) and CD28 (1:500) activating antibodies (ThermoFisher Scientific) in serum-free RPMI for 20 minutes, 37 C. Activated CFSE-labeled T cells were seeded either alone or with: (i) M-CSF-treated BMDMs, (ii) oTME CM-educate BMDMs, or (iii) co-cultured with oTME/BMDMs in DMEM growth media contained 10% FBS (Gibco), 1% P/S. Five days later, CD8+ and CD4+ cell division were analyzed by flow cytometry by quantifying the CFSE dye dilutions.

Immunohistochemistry: Mice were anesthetized with xylazine/ketamine and transcardially perfused with 30 mL cold PBS, followed by 10 mL cold 4% PFA. Tissues were collected and fixed in 4% PFA overnight, washed, transferred to 70% Ethanol, and embedded in paraffin blocks. Paraffin sections (10-12 μm) were mounted on plus slides, de-waxed in xylene and hydrated into graded alcohol solutions. Endogenous peroxidase activity was quenched by immersing the slides in 1% hydrogen peroxide in PBS for 15 minutes, room temp (RT). Antigen retrieval pretreatment was performed in a steamer using the appropriate buffer for 30 minutes. Sections were incubated overnight with primary antibody in 4° C. For DAB staining, sections were washed with PBS and incubated with the appropriate secondary antibody followed by avidin-biotin complexes (Vector Laboratories, Burlingame, CA, Cat. No. PK-6100). Antibody reaction was visualized with 3-3′ Diaminobenzidine (Sigma, Cat. No. D8001) followed by counterstaining with hematoxylin. Tissue sections were dehydrated in graded alcohols, cleared in xylene and sealed with coverslips. For immunofluorescence staining, slides were stained fluorescently-labelled secondary antibodies (Invitrogen) for 1 hr at room temperature, counterstained with DAPI (5 m/mL) for 5 min, washed and sealed with VECTASHIELD® Antifade Mounting media.

Immunofluorescence and Image Processing: Cells were grown on coverslips for 48 hours. After treatment, cells were washed, permeabilized using 0.02% Triton X-100 and fixed for 20 min with 4% PFA. Cells were blocked with 5% BSA and incubation with primary antibodies was overnight in 4° C. Probing with AlexaFluor-488, AlexaFluor-555, or AlexaFluor-647 fluorescent secondary antibodies (Invitrogen) was carried out for 1 hr at room temperature. Slides were sealed (VECTASHIELD® Antifade; H-1000) and images were taken using an inverted fluorescence Nikon microscope, or LSM880 Zeiss confocal microscope. Images were processed using Photoshop (version 21.1.2) and analyzed by Fiji/ImageJ (version 2.1.0) software.

Immunoblotting Analysis: Cell lysates were collected, cleared and processed as previously described (Ben-Chetrit et al., “Synaptojanin 2 is a Druggable Mediator of Metastasis and the Gene is Overexpressed and Amplified in Breast Cancer,” Sci. Signal. 7:8 (2015), which is hereby incorporated by reference in its entirety). Samples were loaded on Mini-PROTEAN®Precast 4-15% gradient gels (BioRad; 456-1083). Antibodies against ARG1 (#79404), Notch Activated Targets Antibody Sampler Kit (#68309), phospho-SMAD2/3 (#8685), CSF-1R (#3152), phospho-AKT Ser473 (#4060), AKT (#9272), phospho-ERK (#9101) were purchased from Cell Signaling.

Cytokine Arrays: Mouse XL Cytokine Arrays were purchased from R&D Systems and used according to the manufacturer's instructions. Briefly, similar volumes of conditioned media were collected, cell debris were removed by centrifugation (2000 g, 10 min 4C), and cleared supernatants were loaded on spotted membranes for 16 hrs, 4° C. with tilting. Secreted cytokines, chemokines and growth factors (111 in total) were probed in duplicated along with positive and negative controls.

### Quantification and Statistical Analysis of Examples 1-8

Parameters such as sample size, precision (mean±SD or SEM), and statistical test significance are reported in the Examples, Figures, and Figure Legends. Statistical significance was calculated by one-way ANOVA when comparing two groups or two-way ANOVA when comparing three or more groups. A p<0.05 was considered statistically significant. Prism v8 software was used for statistical data analysis.

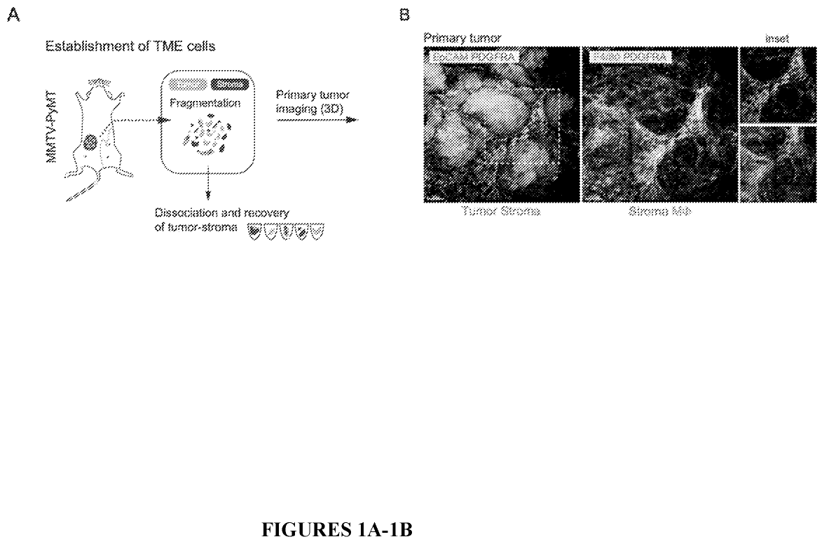

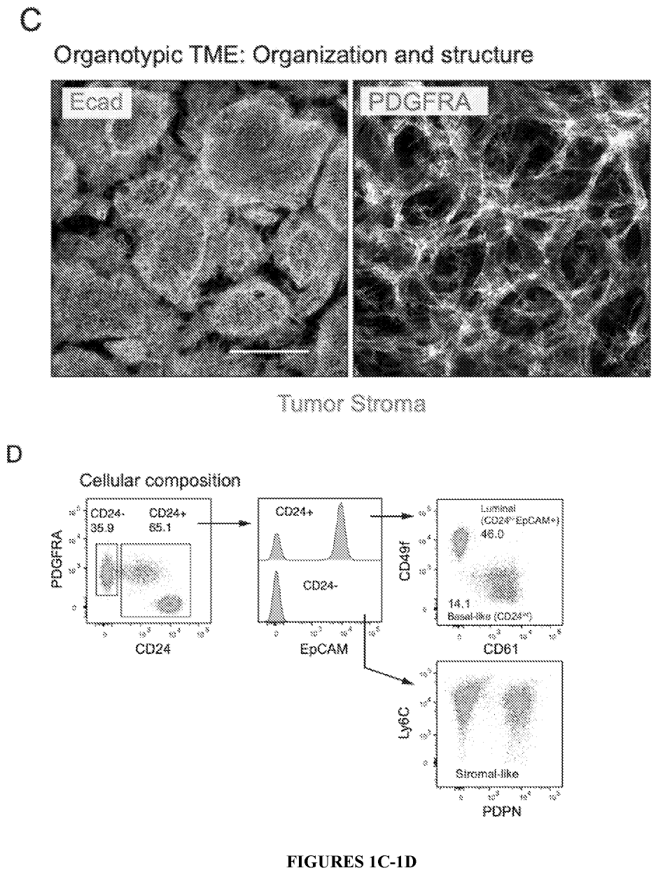

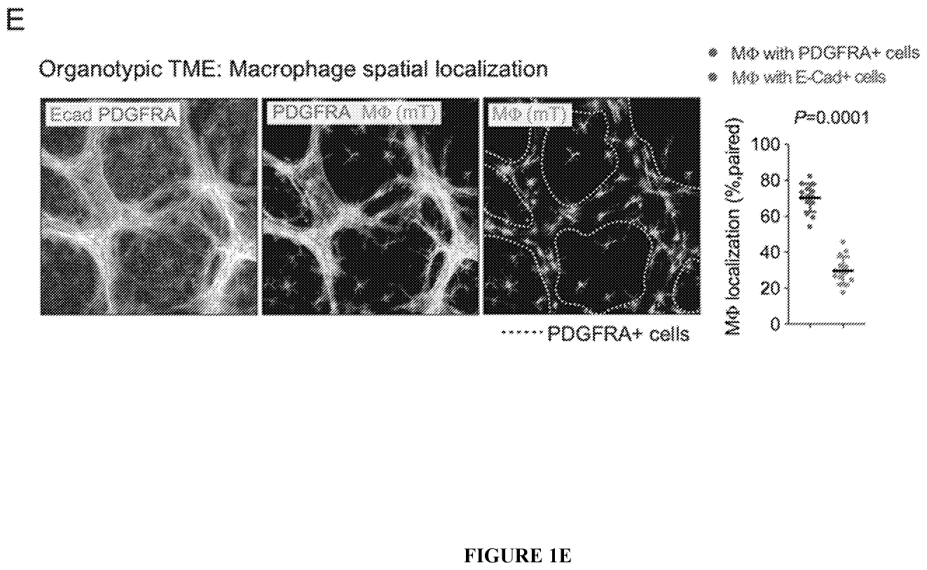

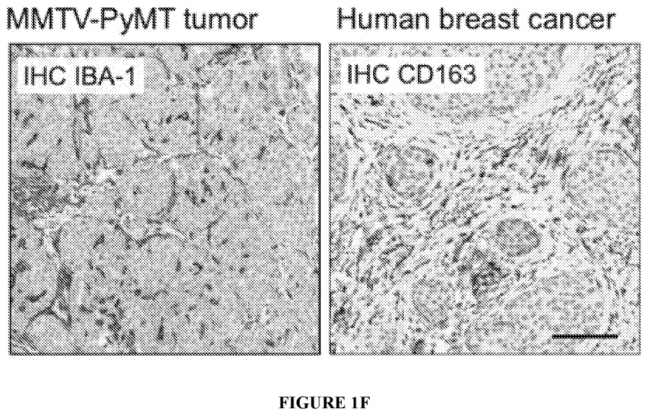

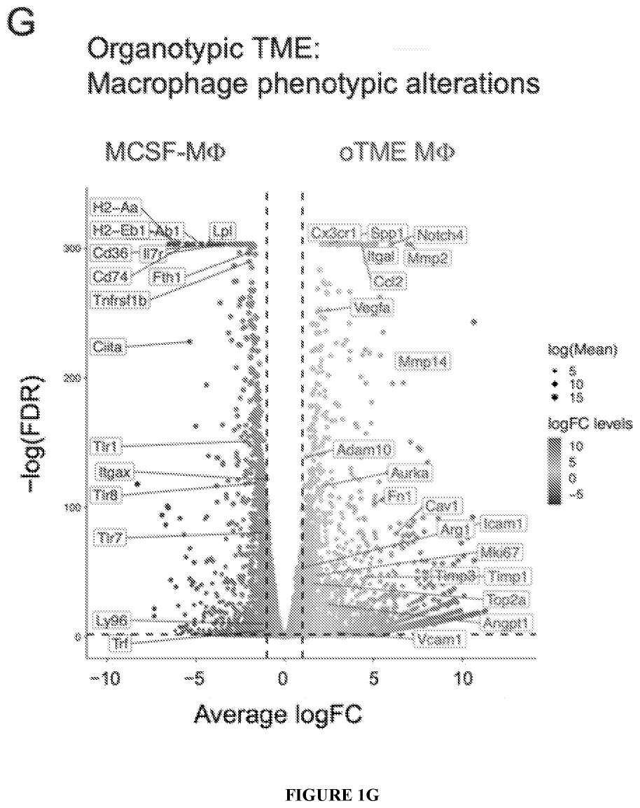

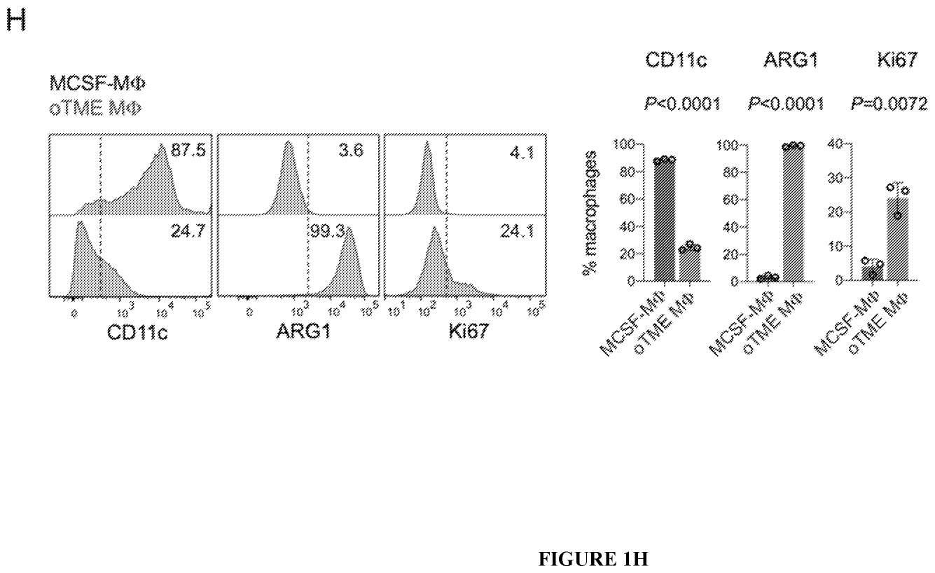

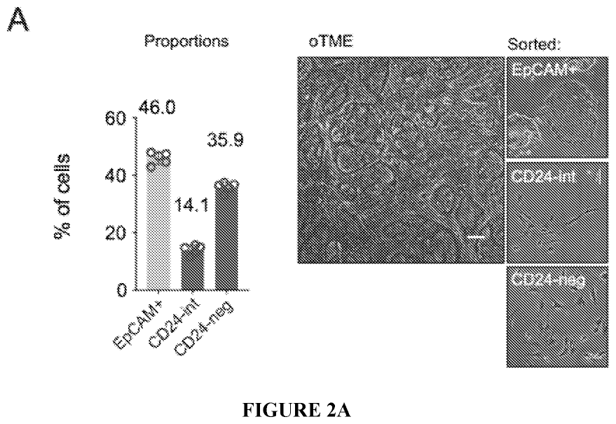

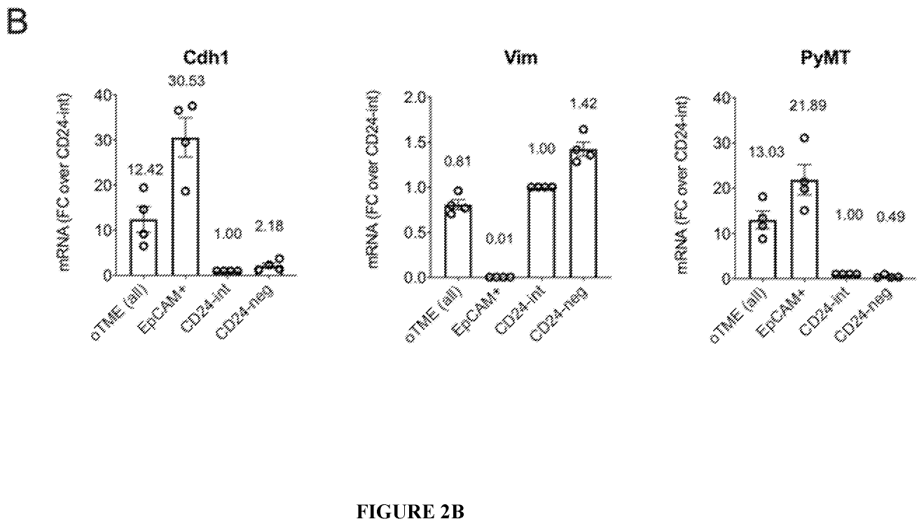

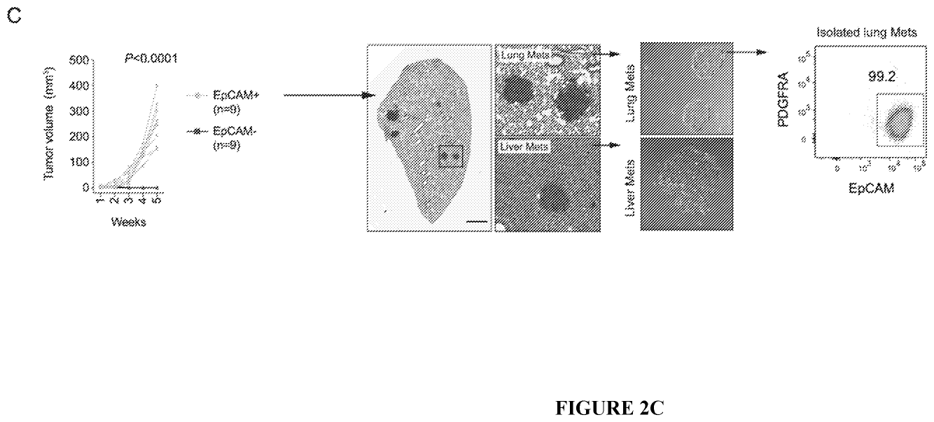

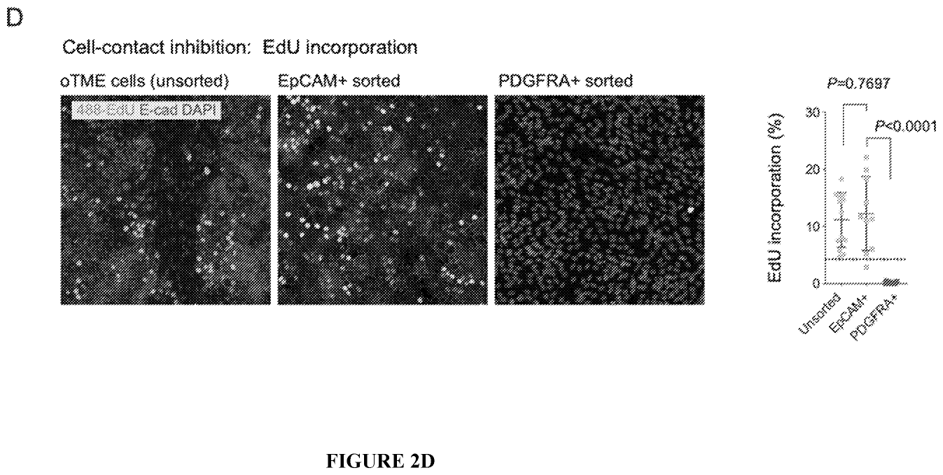

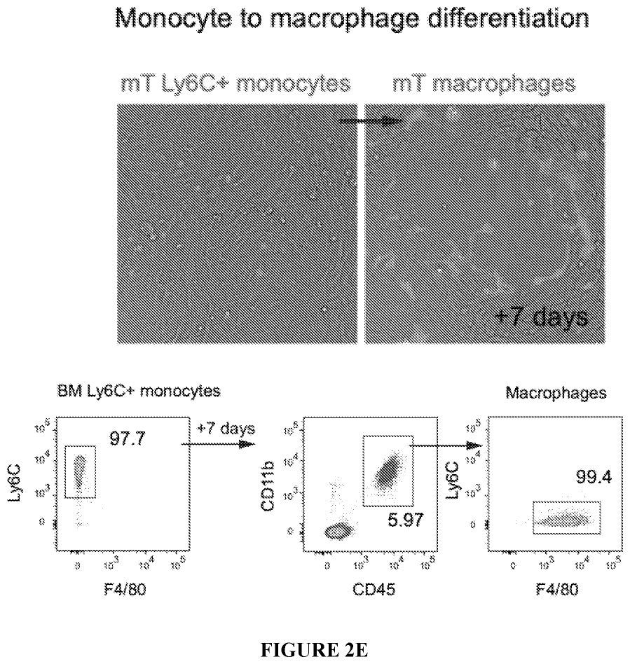

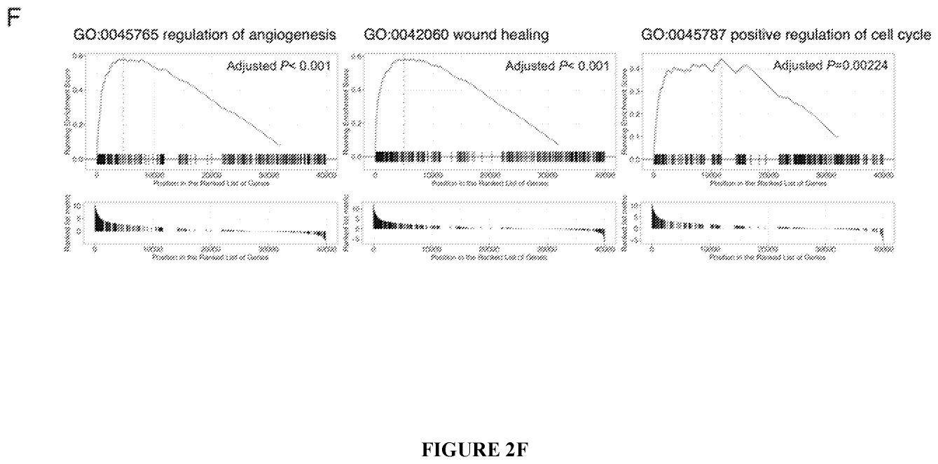

Bulk RNA-Seq Analysis: BMDMs were co-cultured with oTME cells (educated) or left alone with M-CSF for 10 days (n=3 replicates, each). Then, BMDMs were FACS-sorted and RNA was extracted using Trizol-LS. RNA-sequencing libraries were generated with the SMART-Seq preparation kit (CloneTech) with the Nextera XT kit (Illumina), and pair-end 150-bp sequencing was performed by GeneWiz (South Plainfield, New Jersey, USA) on an Illumina HiSeq 2500. FASTQ files were mapped to the mouse genome (mm 10) using STAR (version 2.5.3a) with default parameters (Dobin et al., “STAR: Ultrafast Universal RNA-Seq Aligner,” Bioinformatics 29:15-21 (2013), which is hereby incorporated by reference in its entirety). Transcript count was quantified using STAR -quantMode option with Gencode mouse release M21 annotation GTF files (https://www.gencodegenes.org/mouse/release_M21.html). The resulting count matrix was analyzed and normalized using DESeq2 v1.18.1 (Love et al., “Moderated Estimation of Fold Change and Dispersion for RNA-Seq Data with DESeq2,” Genome Biol. 15:550 (2014), which is hereby incorporated by reference in its entirety). Differential gene expression was assessed with default parameters. Differentially expressed genes were defined as any gene with an absolute log-fold change (logFC) larger than 1 at a false discovery rate (FDR) of 0.01. To generate the volcano plot in FIG. 1G, genes that have NA logFC values were removed, and for the genes that have p values of zero, the -log10 p values were set to 0.1 times the smallest non-zero p-value. Gene Set Enrichment Analysis of Gene Ontology was performed using the gseGO function in the clusterProfiler v3.16.0 R package (Yu et al., “ClusterProfiler: An R Package for Comparing Biological Themes Among Gene Clusters,” OMICS 16:284-287 (2012), which is hereby incorporated by reference in its entirety) with 10000 permutations and other default parameters. The Gene Set Enrichment Analysis results showing the significantly enriched (FDR<0.05) GO terms is provided in FIG. 2F. Next-Generation Sequencing of Guide Abundance Readout: The inventors

sequenced guide abundances after gDNA extraction and PCR of the guide cassette on an Illumina MiSeq 150 cycle v3 with ˜25 million reads. The inventors aimed for 50 reads per sgRNA within each time-point. Resulting FASTQs were trimmed using cutadapt (version 0.12.0) to result in 20 bp sgRNA sequences in two steps. First, the 5′ flanking regions of the 20 bp sgRNA were trimmed off using the recognition sequence GACGAAACACCG (SEQ ID NO: 12) directly 5′ of the sgRNA with a maximum allowable error rate of 0.2 or 20% of base pairs. The trimmed sequences were trimmed again for the 3′ sequence flanking the 20 bp sgRNA using GTTTAAGAGCTA (SEQ ID NO:13) as a recognition sequence, a maximum allowable error rate of 0.2, and a minimum read length of 5 bp. This yielded 20 bp sgRNA sequences. To derive a read count frequency for each of the sgRNAs in the CRISPR library, the reads were aligned to the library using bowtie. A bowtie index for the CRISPR library was constructed using the bowtie-build command on a FASTA file where each sgRNA sequence was a FASTA entry. Bowtie was used to align the trimmed reads to the reference, with arguments −v 1 to allow for only 1 bp mismatch and −m 1 to only keep unique alignments. Unique sgRNAs were counted from the alignment to produce a sgRNA read count table, which is used for all downstream analysis. To ensure an evenly distributed sgRNA representation at the initial, unselected time-point, the inventors calculated a skew, where skew is defined by dividing the 90% read count quantile by the 10% read count quantile, and aimed for a skew no greater than 2. The samples which were taken from the initial library representation were close to 2, while the Arg1-EYFP+ and Arg1-EYFP^{Neg} sorted groups were close to 20. This was expected as sgRNAs that were abundant in Arg1-EYFP+ are expected to have lower abundance or absence in Arg1-EYFP^{Neg}. Two biological replicates (RepA and RepB) of the M2-education CRISPR screen were generated from two batches of mice and each biological replicate was further split into two technical replicates. The inventors sequenced and processed them separately and combined the technical replicates together to generate one raw count matrix containing the two biological replicates.

Analysis of M2-Education CRISPR Screen: The MAGeCK v0.5.8 software (Li et al., “MAGeCK Enables Robust Identification of Essential Genes From Genome-Scale CRISPR/Cas9 Knockout Screens,” Genome Biol. 15:554 (2014), which is hereby incorporated by reference in its entirety) test module was used to identify negatively and positively enriched screen hits. Briefly, the sgRNA counts were normalized using the size factor estimated from the 1000 control (non-targeting) guides. The p-value for each gene is tested by 50,000 rounds of randomized permutation, and adjusted with Benjamini—Hochberg procedure. Log2 fold change (LFC) for each gene is calculated using the second-best sgRNA. MAGeCK produced both guide-level and gene-level enrichment scores using the alpha-robust rank aggregation (RRA). The RRA was specified to consider the top 0.1 percentile of the guides as successful targeting sgRNA and genes that have at least one successful targeting sgRNAs were selected.

Chromium 10× Single-Cell RNA-Seq: Single-cell RNA-sequencing data generated with 10× Genomic Chromium Single Cell 3′ Kit v2 (10× Genomics) and were processed using Cell Ranger (v1.3.1) with default parameters. Samples were sequenced at an average of 50,000 reads per cell. Raw sequencing data were demultiplexed and post-processed following the custom pipelines provided by 10× Genomics. Briefly, raw base calls were demultiplexed into fastq files using the cellranger mkfastq command, followed by alignment to the selected reference mm10 genome. Barcode and UMI counting were performed using the cellranger count command with default parameters.

### Example 1

### Single Cell RNA-Seq Analysis

R markdown HTML documents for the scRNA-seq analysis are provided as the Supplementary Note and corresponding scripts can be downloaded from the Github repository. All six samples were analyzed together as described in the following sections. The oTME cells that were cultured alone (2 and 10-days) were excluded for visualization purposes in this paper.

Preprocessing: The count matrices of all six samples were pooled and normalized using log-transformed transcript per million (logTPM). Specifically, the inventors denoted the UMIcount of jₜₕ gene in iₜₕ cell as Count_{i,j}, and the logTPM was calculated as

$$log {TPM}_{i , j} = log \frac{{Count}_{i , j}}{{10}^{4} \times {\sum }_{j} {Count}_{i , j}} + 1.$$

The Seurat v2.3.4 R package (Butler et al., “Integrating Single-Cell Transcriptomic Data Across Different Conditions, Technologies, and Species,” Nat. Biotechnol. 36, 411-420 (2018), which is hereby incorporated by reference in its entirety) was used for downstream analysis. Low-quality cells with less than 500 genes detected or mitochondrial gene percentage >10% were filtered out. Genes that were expressed in less than 50 cells were also removed. After filtering, the 19,280 cells in total were retained with an average of 13,013±56.49 UMIs per cell (mean±s.e.m.) and an average of 2,947±7.49 (mean±s.e.m.) genes detected across all cells. A set of 1,000 highly variable genes were identified using the FindVariableGenes function with default parameters, which finds variable genes while controlling for the strong relationship between variability and average expression.

Dimensionality reduction: The normalized count data was scaled and centered using ScaleData function with default parameters to calculate z-score for each gene. Principal Component Analysis (PCA) was performed using the RunPCA function with the 1,000 highly variable genes identified from the preprocessing step. The first 15 PCs were selected for downstream clustering and dimensionality reduction analysis based on the observation of the PC “elbow” using the PCElbowPlot function. tSNE (Maaten and Hinton, “Visualizing Data Using t-SNE,” J. Mach. Learn. Res. 9:2579-2605 (2008), which is hereby incorporated by reference in its entirety) was used to visualize the scRNA-seq data. The top 15 PCs were selected and used the RunTSNE function to perform tSNE dimensionality reduction to embed the data into two dimensions. Clustering: The modularity based shared nearest neighbor (SNN) clustering

algorithm was implemented in Seurat's FindClusters function with resolution=1 and other default parameters using the top 15 PCs. Initially the 19 clusters were retrieved. The clustering result were validated by constructing a phylogenetic tree of the 19 clusters using their average expressions. The BuildClusterTree function with the top 15 PCs was used as input to calculate the distance between clusters. The clustering quality was assessed using the AssessNodes function to calculate the out of bag error for a random forest classifier trained on the bottom 25% of the nodes. Nodes with an out of bag error bigger than 0.03 were merged together. The above steps were repeated until all the nodes have an out of bag error below 0.03. One of the clusters expresses heterogeneous lineage markers of epithelial (Epcam), CAF (Fn1, Acta2), and basal-like (Cd24a) cells. It also exhibits higher UMI counts per cell compared with the non-cycling cells. These features are common to doublet/multiplet cells that are considered as artifacts of droplet-based scRNA-seq technologies (DePasquale et al., “DoubletDecon: Deconvoluting Doublets from Single-Cell RNA-Sequencing Data,” Cell Rep. 29(8):1718-1727 (2019) and McGinnis et al., “DoubletFinder: Doublet Detection in Single-Cell RNA Sequencing Data Using Artificial Nearest Neighbors,” (2018), which are hereby incorporated by reference in their entirety). Therefore, this cluster was annotated as doublets and removed it from downstream analysis. Macrophage clusters were taken from the data for further analysis. Cells from one of the clusters expressing Mki67 are the cycling macrophages. They are heterogeneous in that they contain both cycling M-CSF-treated and cycling educated macrophages, but this biological difference was masked by the strong cell cycle effect. Therefore, this cycling population was dissected by following the same procedures as described in the Preprocessing, Dimensionality reduction, and Clustering sections with the top 10 PCs and an out of bag error of 0.07. In addition, the inventors further identified and removed a group of doublet cells of macrophages and CAFs. The macrophages were clustered mainly by education status (M-CSF-treated vs educated), time points (early vs late), and cell cycle phases (non-cycling vs cycling). Thus, they were annotated accordingly as M-CSF-treated CD11c+, M-CSF-treated, M-CSF-treated-cycling, early, early-cycling, late, late-cycling macrophages. In total, 2 clusters of Epcam+tumor cells, 3 clusters of Cd24a+ Epcam− Basal-like cells, 3 clusters of Acta2+ CAFs, and 7 clusters of Cd68+ M-CSF-treated and educated macrophages were identified.

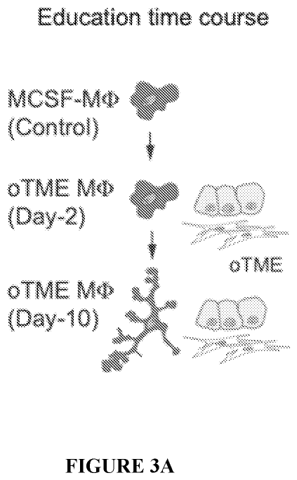

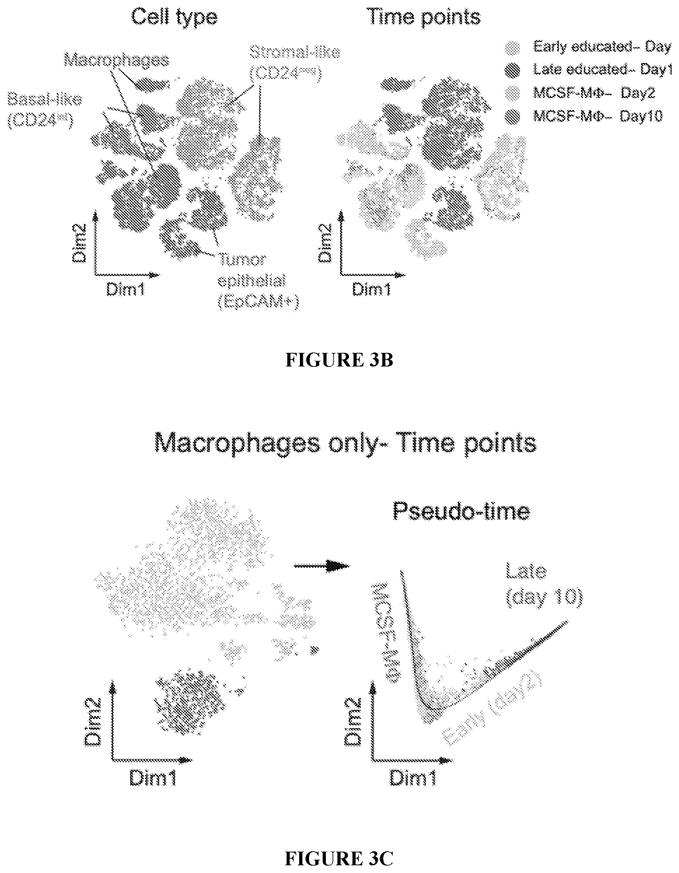

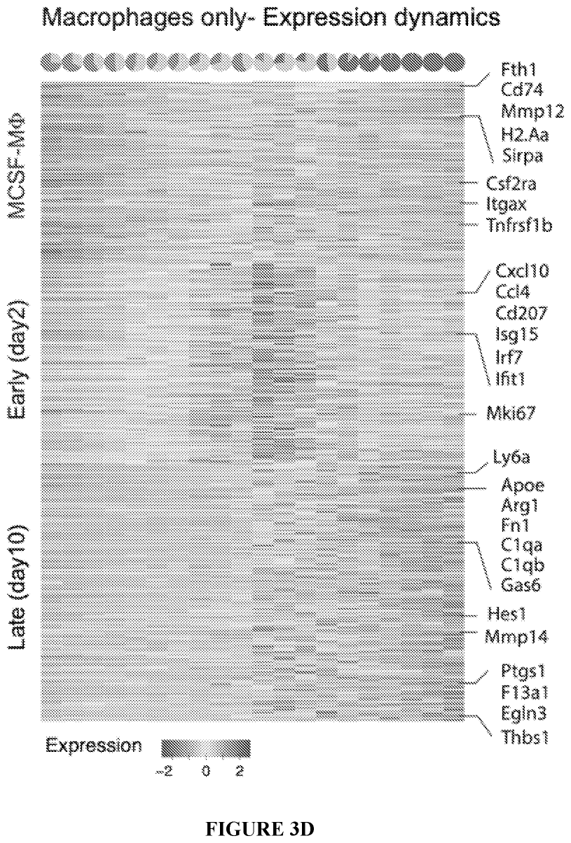

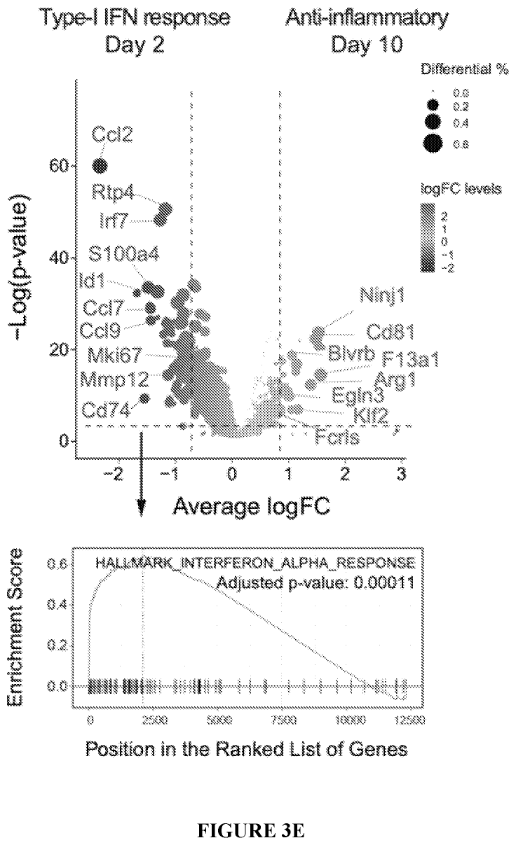

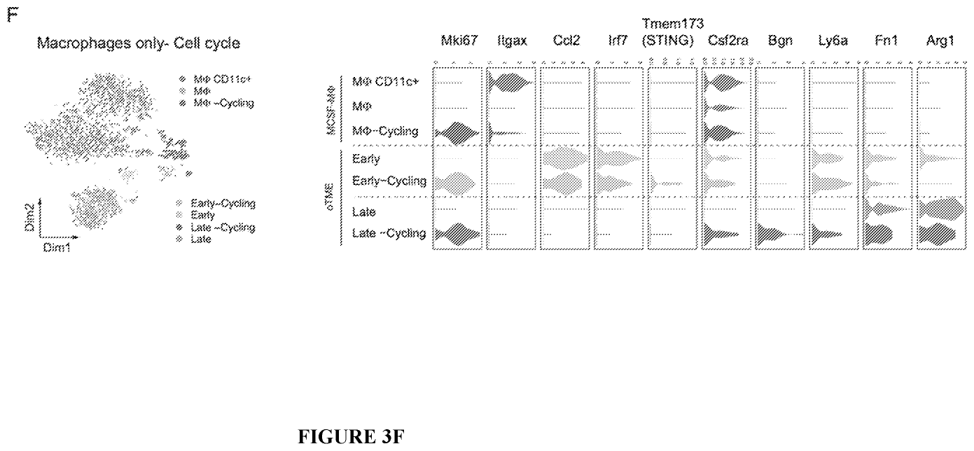

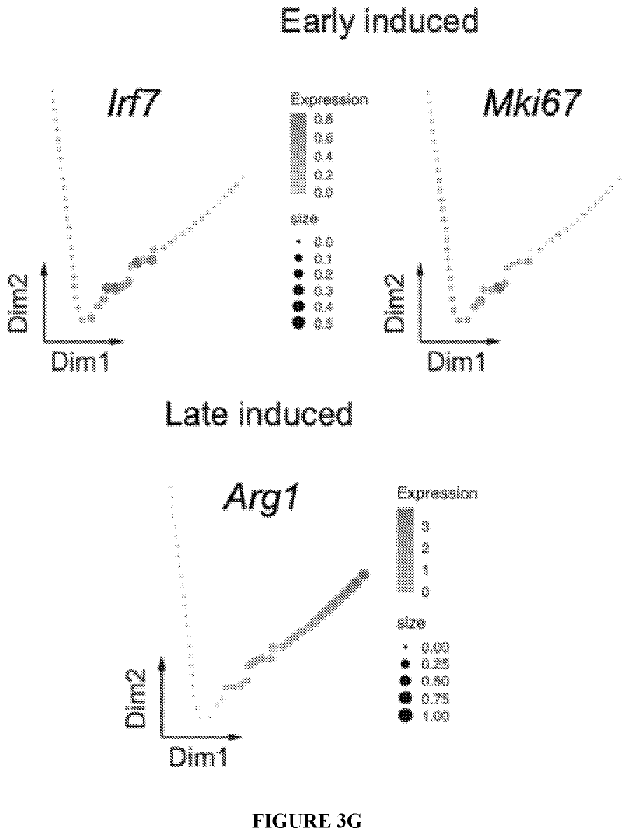

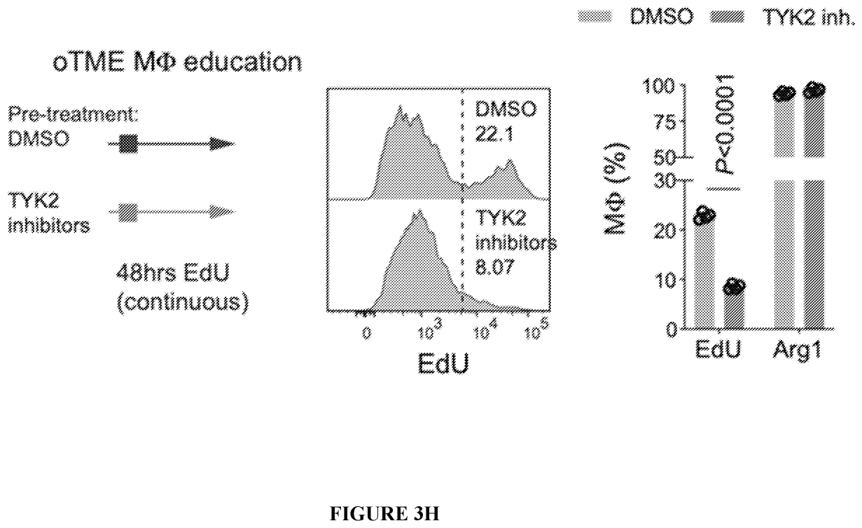

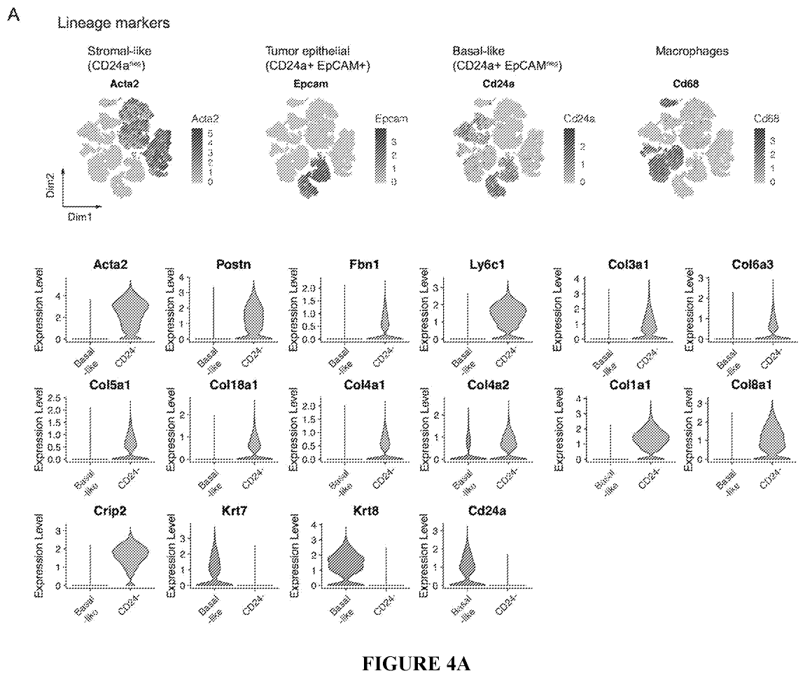

Differential Expression: Differential expression analysis was performed using the Wilcoxon Rank Sum test implemented in Seurat's FindAllMarkers function. Two sets of differential expressed genes were defined. First, 4 cell lineages were defined based on known markers and grouped the clusters into Epcam+ tumor epithelial cells, Acta2+ CAF cells, Cd24+ Epcam− basal-like cells, and Cd68+ macrophages lineages. Differential expression analysis was conducted among these 4 lineages with the parameter min.pct=0.5 which only tests genes that were detected in a minimum percentage of 50% of the cells in each lineage in order to get a consensus list of differential expressed markers. Genes with a log fold change bigger than 1 and a p-value of less than 0.01 are considered as lineage markers. Second, a set of cell-type markers were defined by comparing the clusters within each lineage (i.e., cycling tumor epithelial cells vs all tumor epithelial cells). In addition, non-cycling and cycling macrophage clusters were separately analyzed for differential expression. The parameter min.pct=0.4 was used in order to get subtler differences between similar cell types. Genes with a log fold change greater than 0.75 and a p-value of less than 0.01 are considered as cell-type markers. All the p-values are adjusted in Seurat using Bonferroni correction. To generate the volcano plot in FIG. 3E, all the genes between macrophage populations in early and late time points were compared using Wilcoxon Rank Sum test followed by Benjamini-Hochberg procedure.

Education Trajectory: A diffusion map (Coifman and Lafon, “Diffusion Maps,”Appl. Comput. Harmon Anal. 21:5-30 (2006), which is hereby incorporated by reference in its entirety), a non-linear dimensionality reduction technique, was used to capture the continuous transitions (aka pseudotime) during macrophage education in the scRNA-seq data. Specifically, the differentially expressed markers of M-CSF-treated (Itgax; CD1 1c+) and educated (late) non-cycling macrophage clusters were first identified as described in the Differential expression section but using the MAST method (Finak G., et al., “MAST: A Flexible Statistical Framework for Assessing Transcriptional Changes and Characterizing Heterogeneity in Single-Cell RNA Sequencing Data,” Genome Biol. 16:278 (2015), which is hereby incorporated by reference in its entirety). The inventors then used the scaled and centered macrophage scRNA-seq data of these 74 marker genes as input for the DiffusionMap function in the destiny v2.6.2 R package (Angerer et al., “Destiny: Diffusion Maps for Large-Scale Single-Cell Data in R,” Bioinformatics 32:1241-1243 (2016), which is hereby incorporated by reference in its entirety) with default parameters. The first two eigenvectors (DM1 and DM2) captured the continuous transition between M-CSF-treated and educated macrophages. Therefore, these were used to represent the macrophage education trajectory. Then a principal curve (Hastie and Stuezle, “Principal Curves,” J. Am. Stat. Assoc. 84:502-516 (1989), which is hereby incorporated by reference in its entirety) was fitted using the princurve v2.1.4 R package to the education trajectory (DM1 and DM2). The inventors defined the education pseudotime as projection of each cell onto this principal curve (the arc-length from the beginning of the curve), and normalized the pseudotime into 0-1 range as described previously (Wang et al., “A Single-Cell Transcriptional Roadmap for Cardiopharyngeal Fate Diversification,” Nat. Cell Biol. 21:674-686 (2019), which is hereby incorporated by reference in its entirety). To identify the gene expression dynamics, the gene expression profiles of the 1,000 highly variable were genes smoothed as described above on the education pseudotime by fitting a local polynomial regression, using loess R function with the smoothing parameter span=0.7. The mutual information between the smoothed gene expression profiles and the pseudotime was calculated using the discretize and mutinformation functions in the infotheo v1.2.0 R package (Meyer, “Information-Theoretic Variable Selection and Network Inference From Microarray Data,” Universite Libre de Bruxelles (2008), which is hereby incorporated by reference in its entirety) with default parameters. The results were filtered by removing the genes that have lower mutual information than the 25% quantile of the total mutual information calculated, and retained 750 genes. K-means clustering was applied using the kmeans R function to cluster these gene expression dynamics into 3 clusters. The 3 clusters of genes correspond to M-CSF-treated, transient (early day 2) and educated (late day 10) macrophage signatures according to their expression dynamics on the education pseudotime. To visualized the gene expression dynamics during macrophage education as shown in FIG. 3D and FIG. 3F, the inventors sorted the cells and split them into a number of equal-width bins (n=20 in FIG. 3D; n=50 in FIG. 3F) on the education pseudotime, and average expression profiles were calculated within each pseudotime bin.

Gene Signature Scoring: For a given gene set, the gene signature score in single cells was calculated as described previously (Tirosh et al., “Dissecting the Multicellular Ecosystem of Metastatic Melanoma by Single-Cell RNA-Seq,” Science 352:189-196 (2016), which is hereby incorporated by reference in its entirety). The AddModuleScore function in Seurat was used to calculate the gene signature score. Briefly, signature genes are splitted into 10 bins based on their average expression levels. For each gene, 100 control genes were selected at random within the same expression bin to serve as control sets. The gene signature score was calculated as the differences between the aggregated expression of signature genes and the controls.

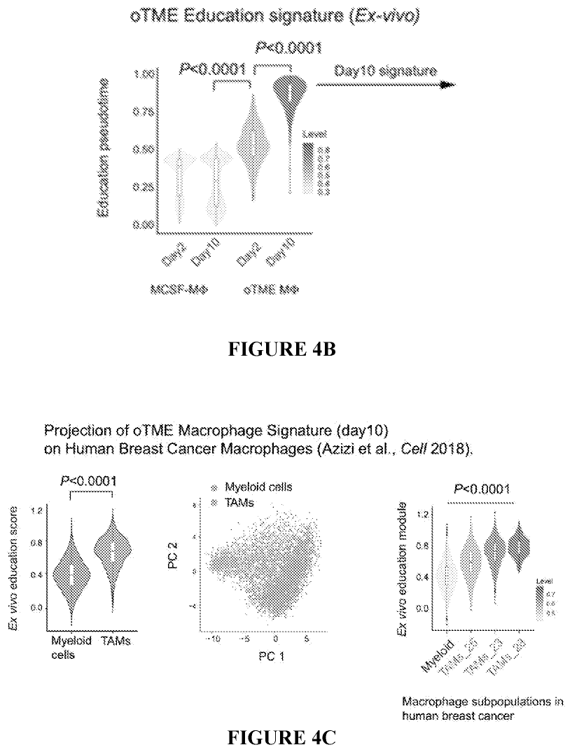

Human Breast Cancer scRNA-Seq: Human breast cancer scRNA-seq data was obtained from GSE114725. The Final Annotation based on bulk combined with differential expressed genes was used as described by Azizi et al., “Single-Cell Map of Diverse Immune Phenotypes in the Breast Tumor Microenvironment,” Cell 174(36):1293-1308 (2018), which is hereby incorporated by reference in its entirety, to select all the monocytic populations including macrophages, monocytes, monocyte precursors, pDCs and mDCs. The inventors normalized the count data to logTPM as described in the Preprocessing section. The inventors defined the ex-vivo education signature as differentially expressed genes between M-CSF-treated (CD11c+) and educated (late) non-cycling macrophages using Wilcoxon Rank Sum test implemented in Seurat's FindAllMarkers function with parameter min.pct=0.4 . Genes with a log fold change bigger than 0.75 and a Bonferroni corrected p-value of less than 0.01 are selected. Similarly, the human M2-signature was defined by performing differential expression analysis between the three TAM clusters (23, 25, 28) with other monocytic populations using the same procedure. To qualitatively visualize the data, the Principal Component Analysis using only the ex-vivo murine signature was performed as shown in FIG. 4C (middle panel). The inventors found the TAMs and other monocytic populations can be largely separated by the first PC using the inventors ex-vivo signature. The gene signature score was calculated using the ex-vivo signatures in this human breast cancer scRNA-seq data as described in the previous section.

### Example 2

### Organotypic TME (oTME) Modeling of Pro-Tumoral Phenotypic Alterations in Breast Cancer Macrophages

The phenotypic transition of macrophages towards a pro-tumorigenic and immune-tolerant phenotype in breast cancer relies on complex interactions with both tumor cells and their supporting stroma (DeNardo et al., “Macrophages as Regulators of Tumour Immunity and Immunotherapy,” Nature Reviews Immunology 19:369-382 (2019), which is hereby incorporated by reference in its entirety). To comprehensively investigate the macrophage-tumor-stroma interactions that fuel this transition, syngeneic breast cancer cells derived from a C57BL/6 MMTV-PyMT tumor model were utilized (FIG. 1A) (Quail et al., “Obesity Alters the Lung Myeloid Cell Landscape to Enhance Breast Cancer Metastasis Through IL5 and GM-CSF,” Nat. Cell Biol. 19:974-987 (2017), which is hereby incorporated by reference in its entirety). Uniquely, this model consists of tumor epithelial cells and mesenchymal cells associated with a stromal-like phenotype (hereafter, this model is referred to as organotypic TME; oTME). In culture, oTME cells grow cohesively to form multilayer tumor nest structures (E-Cad+), surrounded by PDGFRA+cells without the addition of basement membrane proteins, recapitulating the typical organization of mammary tumor nests in the original model (FIG. 1B and FIG. 1C). In murine mammary glands, CD24 marks both luminal (CD24^{high}) and basal (CD24′') epithelial cells, while CD24^{neg} cells are identified as stromal cells (Shackleton et al., “Generation of a Functional Mammary Gland From a Single Stem Cell,” Nature 439:84-88 (2006) and Sleeman et al, “CD24 Staining of Mouse Mammary Gland Cells Defines (2006)Luminal Epithelial, Myoepithelial/Basal and Non-Epithelial Cells,” Breast Cancer Res. 8:R7, which are hereby incorporated by reference in their entirety) or the non-tumor fraction in this tumor model (Drobysheva et al., “Transformation of Enriched Mammary Cell Populations with Polyomavirus Middle T Antigen Influences Tumor Subtype and Metastatic Potential,” Breast Cancer Res. 17:132 (2015), which is hereby incorporated by reference in its entirety). Relying on these established characterizations, flow cytometry analysis incorporating canonical mammary gland markers (FIG. 1D) showed a dominant population of tumor epithelial cells (EpCAM+) that express luminal markers (CD49^{high}CD24^{high}CD61−), and a relatively smaller population of PDGFRA+CD24′ cells with mesenchymal characteristics (CD49^{low}CD61+) (Asselin-Labat et al., “Gata-3 is an Essential Regulator of Mammary-Gland Morphogenesis and Luminal-Cell Differentiation,” Nat. Cell Biol. 9:201-209 (2007) and Lim et al., “Transcriptome Analyses of Mouse and Human Mammary Cell Subpopulations Reveal Multiple Conserved Genes and Pathways,” Breast Cancer Res. 12:R21 (2010), which are hereby incorporated by reference in their entirety) (FIG. 2A). Within the CD24^{neg} fraction, the inventors identified two main populations that express PDGFRA+CD34+ Ly6C+PDPN+ or PDPN− that were previously reported as markers of the inflammatory subset in breast cancer fibroblasts (Bartoschek et al., “Spatially and Functionally Distinct Subclasses of Breast Cancer-associated Fibroblasts Revealed by Single Cell RNA Sequencing,” Nat. Commun. 9:5150 (2018); Friedman et al., “Cancer-associated Fibroblast Compositions Change with Breast Cancer Progression Linking the Ratio of S100A4 and PDPN Cafs to Clinical Outcome,” Nature Cancer 1:692-708 (2020); and Wu et al., “Stromal Cell Diversity Associated With Immune Evasion in Human Triple-Negative Breast Cancer,” EMBO J. e104063 (2020), which are hereby incorporated by reference in their entirety). Although the CD24^{neg} cells (hereafter referred to as ‘stromal-like’) are immortalized, only tumor epithelial cells expressed high levels of the Polyomavirus middle-T antigen transgene (PyMT) that confers tumorigenesis in the original transgenic model (FIG. 2B). Consistently, the inventors found that only tumor epithelial cells (EpCAM⁺) were transformed as reflected by a loss of contact growth inhibition and the ability to form primary and metastatic lesions in immunocompetent C57BL/6 mice (FIG. 2C-D). Thus, the oTME model includes the tumor epithelial, basal-like and stromal-like components, closely recapitulating the major non-immune cellular constituents of the original in vivo model.

Tissue fibroblasts are heterogeneous cells that arise from diverse origins. During wound-healing responses, including breast cancer, bone marrow-derived mesenchymal/progenitor cells (MSCs) infiltrate the primary tumors and differentiate into a distinct subpopulation of cancer-associated fibroblasts.

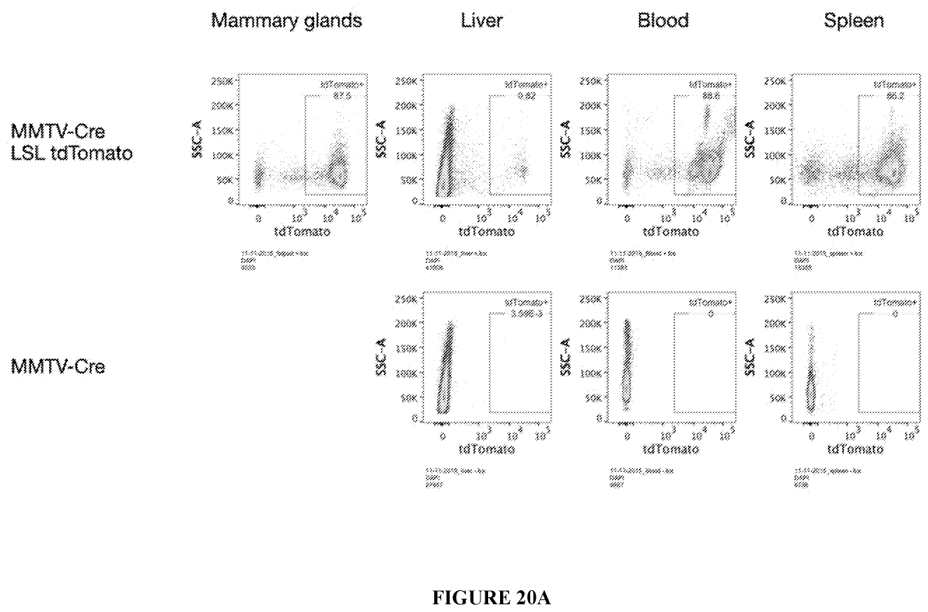

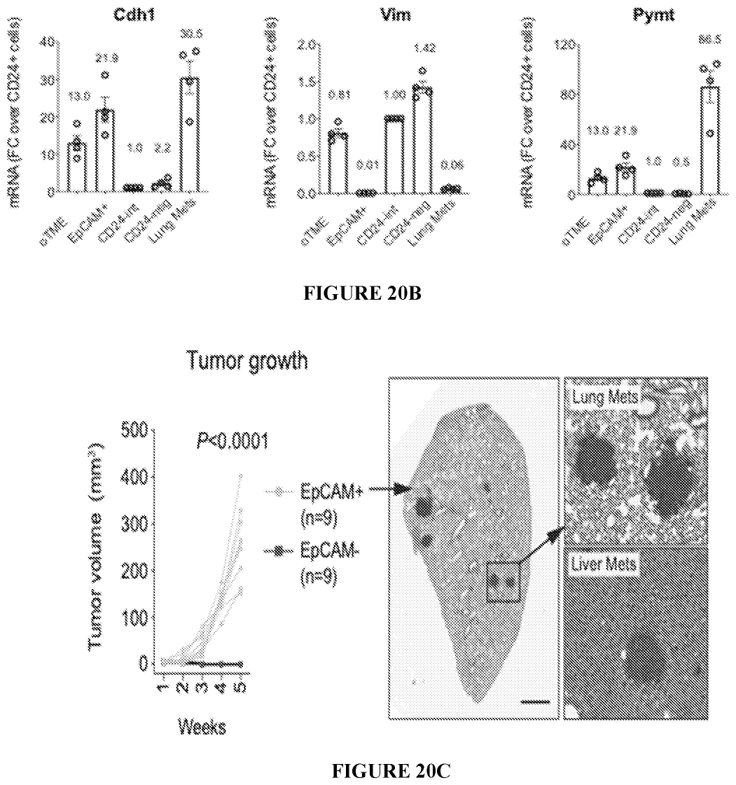

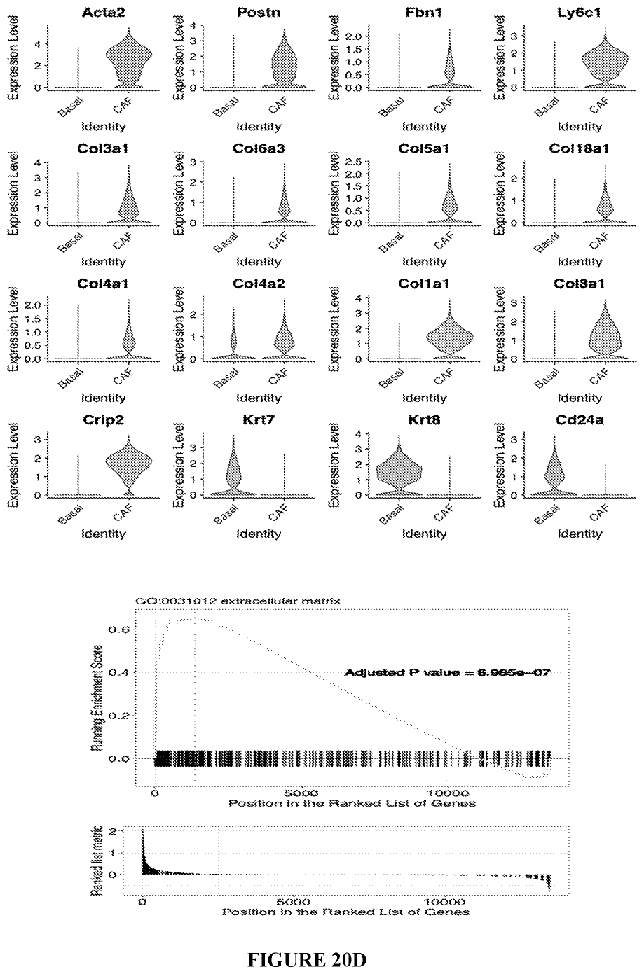

The oTME cells were isolated from a primary tumor of the MMTV-PyMT model that carries the PyMT viral oncogene under the mouse mammary tumor virus (MMTV) promoter. Although the MMTV promoter is active primarily in mammary gland epithelium, the data herein suggest a substantial “leakiness” leading to activation of MMTV promoter in other tissues. MMTV-Cre mice were crossed with LSL-tdTomato reporter mice and the expression of tdTomato in various tissues was analyzed by flow cytometry (FIG. 20A). Surprisingly, tdTomato expression was detected in 88% of blood cells, indicating that bone marrow cells express the PyMT oncogene in the MMTV-PyMT model.

PyMT expression in oTME subpopulations was compared by qPCR and the highest levels were detected in tumor epithelial cells (EpCAM+), but detectable levels were also found in stromal-like cells (PDGFRA+CD24^{neg}) (FIG. 20B).

Importantly, unlike other highly tumorigenic fibroblast lines like the NIH-3T3, the PDGFRA⁺CD24^{neg} stromal-like cells are not transformed (FIG. 20C) but express key lineage and functional markers of fibroblasts (FIG. 20D).

Next, the inventors sought to evaluate whether macrophages cultured in the oTME would recapitulate the spatial localization and tumor-supportive phenotypes observed in breast cancer including, promotion of angiogenesis, matrix remodeling (Azizi et al., “Single-Cell Map of Diverse Immune Phenotypes in the Breast Tumor Microenvironment,” Cell 174(36):1293-1308 (2018); DeNardo et al., “Macrophages as Regulators of Tumour Immunity and Immunotherapy,” Nature Reviews Immunology 19:369-382 (2019); Wagner et al., “A Single-Cell Atlas of the Tumor and Immune Ecosystem of Human Breast Cancer,” Cell 177(18)1330-1345 (2019), which are hereby incorporated by reference in their entirety). To enable visualization of spatial localization, purified Ly6C^{high} bone marrow (BM) monocytes from Rosa26^{mTmG} mice that express membrane-tagged tdTomato (mT) were added (Muzumdar et al., “A Global Double-Fluorescent Cre Reporter Mouse,” Genesis 45:593-605 (2007), which is hereby incorporated by reference in its entirety) (FIG. 2E; left). Flow cytometry confirmed the conversion of Ly6C^{High}F4/80^{neg} monocytes into Ly6C^{neg}F4/80+ macrophages (FIG. 2E, right), indicating that monocytes in this model differentiate into mature macrophages rather than to monocytic myeloid-derived suppressor cells (M-MDSC; Ly6C+F4/80+) (Gabrilovich, “Myeloid-Derived Suppressor Cells” Cancer Immunol Res. 5:3-8 (2017), which is hereby incorporated by reference in its entirety). Immunofluorescence analysis with epithelial (E-cadherin; E-cad) and stromal (PDGFRA) markers showed that macrophages were preferentially co-localized with PDGFRA⁺ cells rather than in E-cad+tumor epithelial nests (70.3% vs. 29.7% respectively; P=0.001, two-tailed paired Wilcoxon test) (FIG. 1E), recapitulating the spatial distribution of macrophages in murine (IBAl; MMTV-PyMT) and human (CD163) breast cancer (FIG. 1F).

To further interrogate the changes in macrophage phenotypes driven by the oTME system, RNA-seq analysis on bone marrow-derived macrophages (BMDMs) cultured for ten days in oTME was performed and compared to M-CSF-treated macrophages left unperturbed as control cells. Differential expression analysis (FIG. 1G) revealed significant phenotypic alterations characterized by downregulation of genes associated with pro-inflammatory activation (Tnfrsf1b, Ly96, T1r1, T1r8, T1r7, Mmp12) (Kratochvill et al., “TNF Counterbalances the Emergence of M2 Tumor Macrophages,” Cell Rep. 12:1902-1914 (2015) and Marchant et al., “A New Transcriptional Role for Matrix Metalloproteinase-12 in Antiviral Immunity,” Nat. Med. 20:493-502 (2014), which are hereby incorporated by reference in their entirety), antigen presentation (H2-Aa, Itgax, Ciita, Cd74), and iron storage (Trf, Fth1) (Leftin et al., “Imaging Endogenous Macrophage Iron Deposits Reveals a Metabolic Biomarker of Polarized Tumor Macrophage Infiltration and Response to CSF1R Breast Cancer Immunotherapy,” Sci. Rep. 9:857 (2019), which is hereby incorporated by reference in its entirety). These changes were coupled with robust induction of genes associated with wound healing and tissue repair (Eming et al., “Inflammation and Metabolism in Tissue Repair and Regeneration,” Science 356:1026-1030 (2017), which is hereby incorporated by reference in its entirety) including, promotion of angiogenesis (Vefga, Egln3, and Cav1), cell-cycle (Mki67, Top2a, and Aurka), scavenging (MerTK, Msr1, Tyro3), and ECM remodeling (Adam10, Mmp14, Fn1, Spp1, and Timp3) (Gene Set Enrichment Analysis; GSEA) (FIG. 2F). Flow cytometry of oTME macrophages confirmed the transition from a non-proliferative pro-inflammatory Ki67^{Neg}CD11c^{hi}Arg1^{Neg} phenotype into a proliferative and immunosuppressive Ki67 + CD11c^{low}Arg1⁺ phenotype (FIG. 1H and FIG. 1I) (Biswas ad Mantovani, “Macrophage Plasticity and Interaction with Lymphocyte Subsets: Cancer as a Paradigm,” Nat. Immunol. 11:889-896 (2010) and Colegio et al., “Functional Polarization of Tumour-associated Macrophages by Tumour-derived Lactic Acid,” Nature 513:559-563 (2014), which are hereby incorporated by reference in their entirety). Of note, while educated macrophages robustly engaged the cell-cycle, the M-CSF-treated macrophages remained predominantly quiescent (FIG. 1G), suggesting that mechanisms other than M-CSF availability license macrophage proliferation in mammary tumors and was successfully captured by the oTME model. Collectively, these results demonstrate that the oTME model faithfully recapitulates the phenotypic alterations of TME macrophages, enabling the high-throughput interrogation of this process.

### Example 3

### Discovery of TME Education Dependencies in Macrophages by Genome-Wide CRISPR/Cas9 Screen

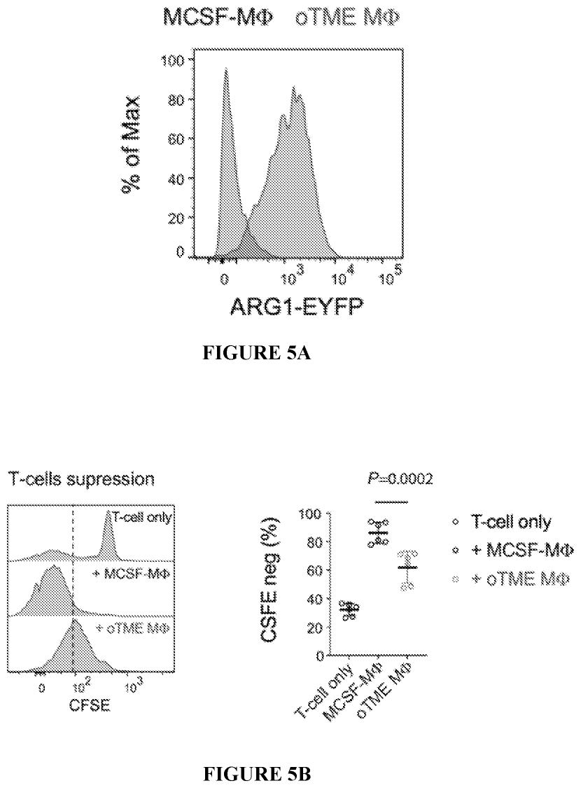

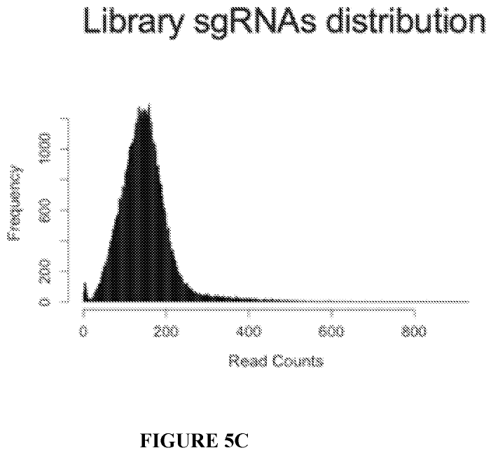

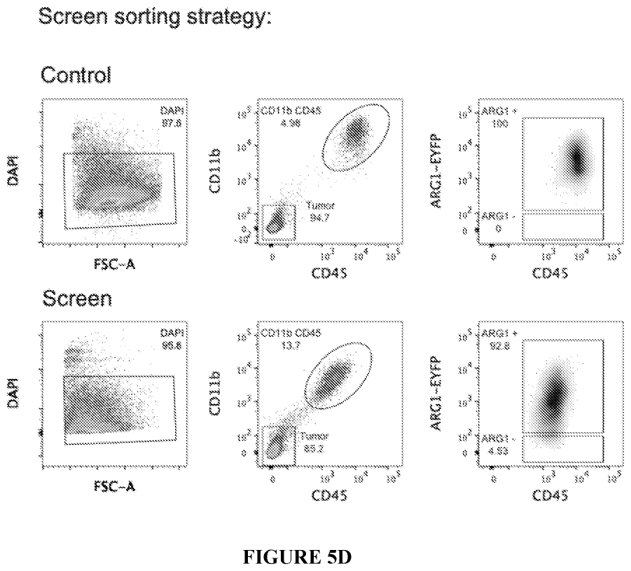

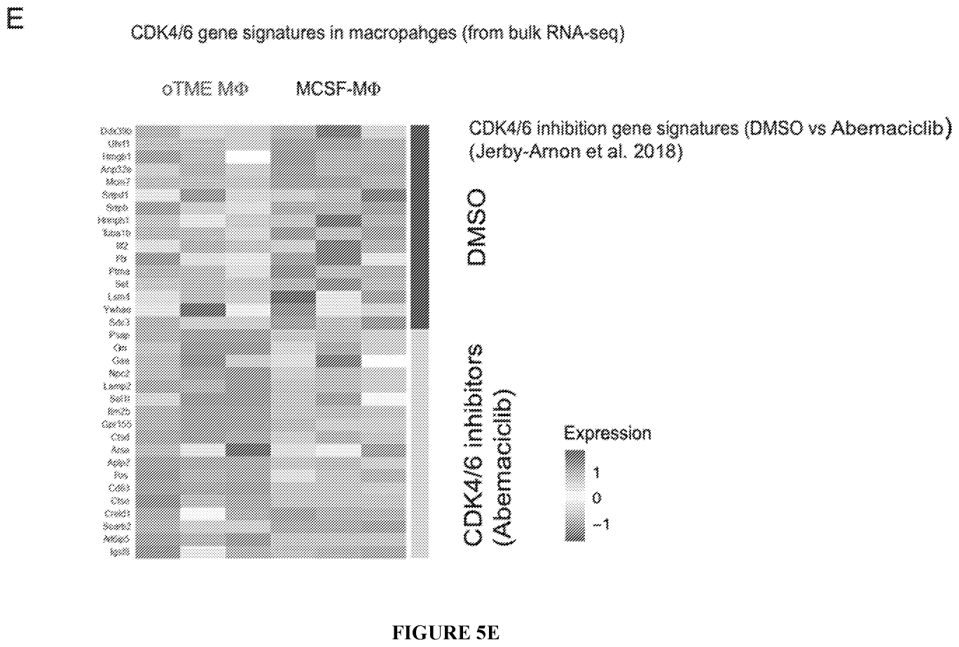

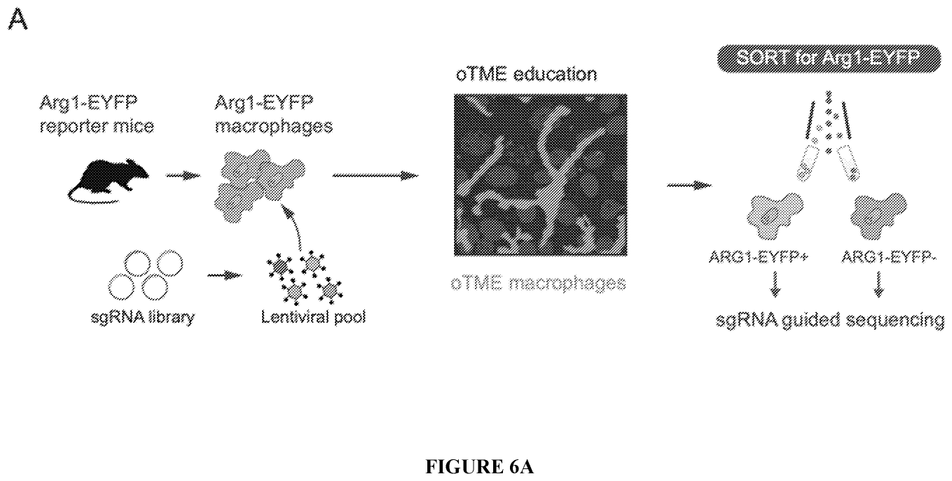

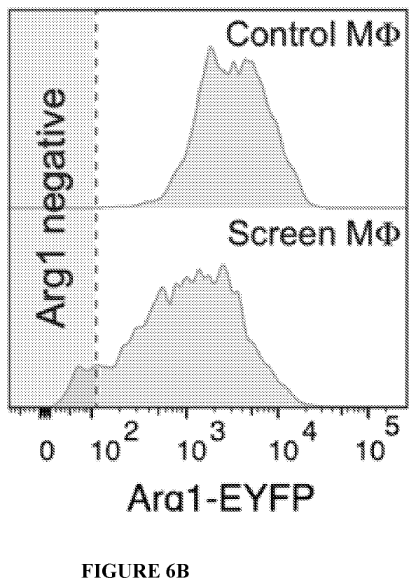

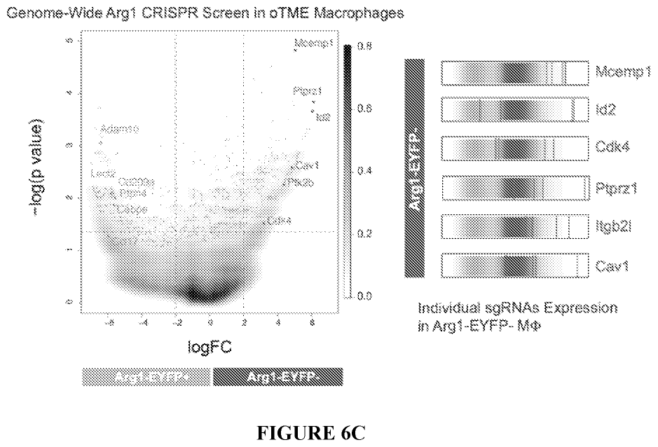

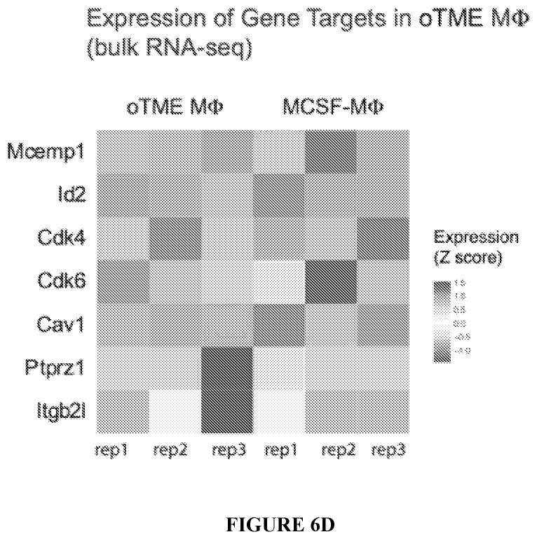

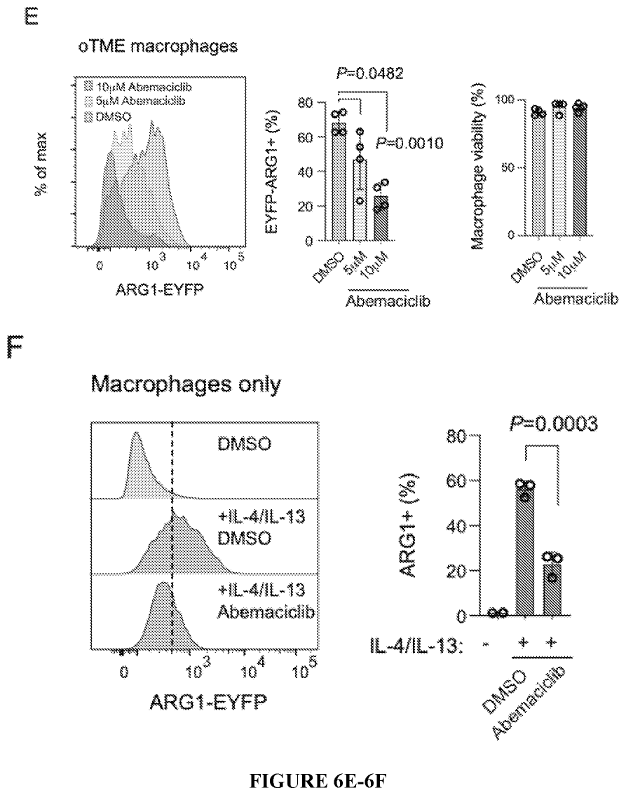

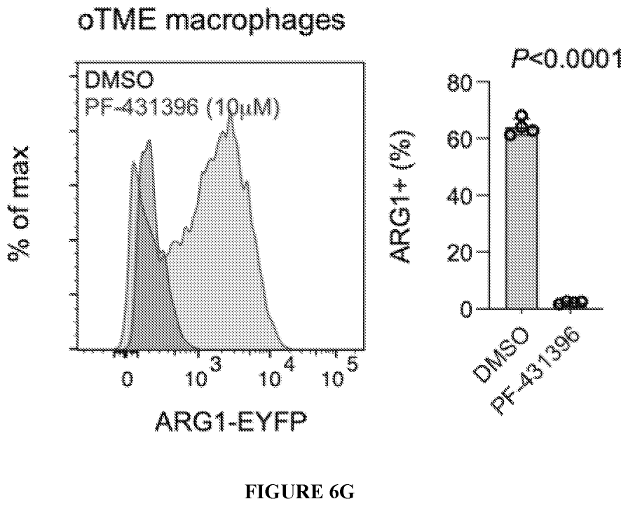

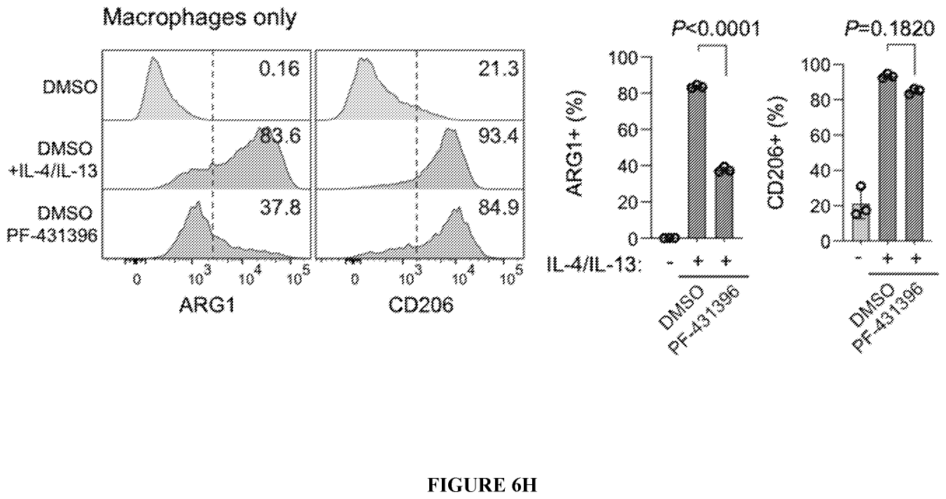

To identify genes essential for TME education of macrophages (often termed M2-like), the inventors leveraged the scalability of the oTME model and performed a genome-wide CRISPR/Cas9 screen in BMDMs. The induction of Arg1 is considered as one of the bona fide hallmarks of M2-like macrophages and associated with anti-inflammatory and tissue repair phenotypes (Biswas ad Mantovani, “Macrophage Plasticity and Interaction with Lymphocyte Subsets: Cancer as a Paradigm,” Nat. Immunol. 11:889-896 (2010); Bosurgi et al., “Macrophage Function in Tissue Repair and Remodeling Requires IL-4 Or IL-13 with Apoptotic Cells,” Science 356:1072-1076 (2017); Colegio et al., “Functional Polarization of Tumour-associated Macrophages by Tumour-derived Lactic Acid,” Nature 513:559-563 (2014), which are hereby incorporated by reference in their entirety). Therefore, Arg1 induction was used as a surrogate for TME-education and screen readout, by utilizing BMDMs from reporter mice that express a yellow fluorescent protein (EYFP) under the control of the Arg1 promoter (Arlauckas et al., “Arg1 Expression Defines Immunosuppressive Subsets of Tumor-Associated Macrophages,” Theranostics 8:5842-5854 (2018) and Reese et al., “Chitin Induces Accumulation in Tissue of Innate Immune Cells Associated with Allergy,” Nature 447:92-96 (2007), which are hereby incorporated by reference in their entirety). First, the induction of EYFP in Arg1-EYFP BMDMs following ten days of culture with oTME cells and their acquired ability to suppress CD8 T cell growth was confirmed (FIG. 5A-B). The inventors cloned whole-genome sgRNA libraries and confirmed a uniform distribution of guides per gene (FIG. 4C; STAR Methods). Next, Arg1-EYFP BMDMs were transduced with sgRNA libraries (aiming for 500 sgRNAs per gene), followed by ten days of co-culture with the oTME system (FIG. 6A). Macrophages were then FACS-sorted based on EYFP expression into EYFP^{neg} (macrophages that failed to induce Arg1) and the top 10% of EYFP^{high} (Argl+macrophages) (FIG. 6B and FIG. 4E). Genomic DNA was extracted and sgRNA abundance was evaluated using targeted DNA sequencing of the sgRNA cassette and MAGeCK package (Li et al., “MAGeCK Enables Robust Identification of Essential Genes From Genome-Scale CRISPR/Cas9 Knockout Screens,” Genome Biol. 15:554 (2014), which is hereby incorporated by reference in its entirety). Through this analysis, gene candidates were defined as sgRNAs that were either enriched in Arg1-EYFP^{neg} cells or depleted in the Arg1-EYFP+ group (FIG. 6C), further intersected with genes that were also expressed in oTME-educated macrophages compared with M-CSF-treated macrophages (RNA-seq in FIG. 1G). These candidates included previously reported mediators of immunomodulatory phenotype in macrophages, including Stat3 (Nakamura et al., “IL10-driven STAT3 Signalling in Senescent Macrophages Promotes Pathological Eye Angiogenesis,” Nat. Commun. 6:7847 (2015) and Yan et al., “STAT3 and STAT6 Signaling Pathways Synergize to Promote Cathepsin Secretion from Macrophages via IRE1α Activation,” Cell Rep. 16:2914-2927 (2016), which are hereby incorporated by reference in their entirety) , the scavenging receptor Marco (Georgoudaki et al., “Reprogramming Tumor-Associated Macrophages by Antibody Targeting Inhibits Cancer Progression and Metastasis,” Cell Rep. 15:2000-2011 (2016), which is hereby incorporated by reference in its entirety), Tnfrsfl2a receptor (TWEAK) that suppress TH1 responses (IL-12) in human macrophages (Maecker et al., “TWEAK Attenuates the Transition From Innate to Adaptive Immunity,” Cell 123:931-944 (2005); Winkles “The TWEAK-Fn14 Cytokine-Receptor Axis: Discovery, Biology and Therapeutic Targeting,” Nat. Rev. Drug Discov. 7:411-425 (2008); and Ye et al., “Enavatuzumab, a Humanized Anti-TWEAK Receptor Monoclonal Antibody, Exerts Antitumor Activity Through Attracting and Activating Innate Immune Effector Cells,” J Immunol Res 2017:5737159 (2017), which are hereby incorporated by reference in their entirety), and Cd200r1 that inhibits the expression of proinflammatory cytokines in myeloid cells including tumor necrosis factor, interferons, and inducible nitric oxide synthase (Liu et al., “CD200R1 Agonist Attenuates Mechanisms of Chronic Disease in a Murine Model of Multiple Sclerosis,” J. Neurosci. 30:2025-2038 (2010); Ritzel et al., “CD200-CD200R1 Inhibitory Signaling Prevents Spontaneous Bacterial Infection and Promotes Resolution of Neuroinflammation and Recovery After Stroke,” J. Neuroinflammation 16:40 (2019); Yoshimura et al., “CD200 and CD200R1 are Differentially Expressed and Have Differential Prognostic Roles in Non-Small Cell Lung Cancer,” Oncoimmunology 9:1746554 (2020), which are hereby incorporated by reference in their entirety) . In addition, several novel candidates including Ptprz1, Itgb21, Id2, Arf6, Ptk2b, Cdk4, and Cdk6 were identified as candidate regulators of the Arg1 induction in macrophages (FIG. 6D).

Notably, CDK4/6 dual inhibitors have been shown to be effective in patients with metastatic breast cancer (Goel et al., “CDK4/6 Inhibition Triggers Anti-Tumour Immunity,” Nature 548:471-475 (2017); Im et al., “Overall Survival with Ribociclib plus Endocrine Therapy in Breast Cancer,” N. Engl. J. Med. 381:307-316 (2019), which are hereby incorporated by reference in their entirety) , acting not only on cancer cells (through cell-cycle arrest) but also unleashing an anti-tumor T-cell-mediated response in mouse models (Deng et al., “CDK4/6 Inhibition Augments Antitumor Immunity by Enhancing T-cell Activation,” Cancer Discov. 8:216-233 (2018), which is hereby incorporated by reference in its entirety). The data herein suggests that CDK4/6 inhibitors may in part trigger anti-tumor immune responses through the disruption of the immunosuppressive phenotype of macrophages in tumors. To further validate CDK4/6 signaling in oTME macrophages, the inventors measured gene signatures that were recently reported with CDK4/6 inhibition (Jerby-Arnon et al., “A Cancer Cell Program Promotes T Cell Exclusion and Resistance to Checkpoint Blockade,” Cell 175(24):984-997 (2018), which is hereby incorporated by reference in its entirety) in the bulk RNA-seq data from oTME vs. M-CSF-treated macrophages. The CDK4/6-associated gene module was predominantly expressed in oTME macrophages, while the CDK4/6 inhibition gene module (generated through treatment with abemaciclib) was enriched in M-CSF-treated macrophages (FIG. 5E). To functionally validate the effect of CDK4/6 inhibition on Arg1 expression, inventors co-cultured Arg1-EYFP BMDMs in the oTME for 10 days and then treated with the CDK4/6 inhibitor abemaciclib or DMSO as control. Abemaciclib-treated macrophages demonstrated a dose-dependent reduction in Arg1-EYFP expression without affecting their viability (FIG. 6E). To further establish a direct effect of CDK4/6 inhibition, macrophages were treated with IL-4/IL-13 to induce Arg1 expression (in the absence of oTME cells) and found that abemaciclib also suppressed Arg1-EYFP expression in this alternative activation mode (FIG. 6F). CDK4/6 inhibition in T-cells resulted in reactivation of the NFAT family of transcription factors that regulate T-cell activation and anti-tumor functions (Schaer et al., “The CDK4/6 Inhibitor Abemaciclib Induces a T Cell Inflamed Tumor Microenvironment and Enhances the Efficacy of PD-L1 Checkpoint Blockade,” Cell Rep. 22:2978-2994 (2018), which is hereby incorporated by reference in its entirety). Interestingly, beyond the upregulation of genes that mediate TH1 response in T-cells, CDK4/6 inhibition also upregulated genes that mediate immunostimulatory responses in antigen-presenting myeloid cells such macrophages and dendritic cells, including MHC-II, Cd86, Ccr2, and Cd40 (Deng et al., “CDK4/6 Inhibition Augments Antitumor Immunity by Enhancing T-cell Activation,” Cancer Discov. 8:216-233 (2018), which is hereby incorporated by reference in its entirety). Together with the screen results, these data suggest that CDK4/6 may act as a molecular switch between immunostimulatory and immunosuppressive phenotypes in TME macrophages.

Another screen candidate and druggable mediator of macrophage education was Ptk2b (Pyk2), a tyrosine kinase previously shown to regulate macrophage inflammasome activation and phagocytosis (Chung et al., “Pyk2 Activates the NLRP3 Inflammasome by Directly Phosphorylating ASC and Contributes to Inflammasome-Dependent Peritonitis,” Scientific Reports 6 (2016); Paone et al., “The Tyrosine Kinase Pyk2 Contributes to Complement-Mediated Phagocytosis in Murine Macrophages,” J. Innate Immun. 8:437-451 (2016); and Rhee et al., “Macrophage Fusion is Controlled by the Cytoplasmic Protein Tyrosine Phosphatase PTP-PEST/PTPN12,” Mol. Cell. Biol. 33:2458-2469 (2013), which are hereby incorporated by reference in their entirety) . Similar to CDK4/6 inhibition, treatment with PTK2B inhibitors (PF-431396) results in robust suppression of Arg1 expression in oTME and IL-4/IL-13-treated macrophages (FIG. 6G and FIG. 6H).

Additional mediators of macrophage education identified in this screen are provided in Table 1 below. These are all druggable mediators. Accordingly, known inhibitors that can be utilized in the methods described herein to inhibit macrophage immunosuppressive phenotypes are also provided.

TABLE 1
Genes Involved in Modulating Macrophage Immunosuppressive Phenotype and Their Known Inhibitors

| | | | | | | | Average | Average | |
|---|---|---|---|---|---|---|---|---|---|
| | | | | | | High | Expression | Expression | |
| | Arg1 | Arg1 | log2 | | | in | Level in | Level in | |
| Gene | EYFP+ | EYFP− | Fold | | | Arg1 | oTME | MCSF | |
| Name | Count | Count | Change | P value | FDR | EYFP− | Macrophage | Macrophage | Known Inhibitor Drug |
| Ppp2ca | 2.0379 | 75.593 | 4.6561 | 2.20E−16 | 1.55E−14 | TRUE | 2494.61 | 2527.25 | LB-100 |
| Psme4 | 14.265 | 127.62 | 3.0748 | 6.34E−16 | 4.31E−14 | TRUE | 1163.65 | 1200.46 | BORTEZOMIB; CARFILZOMIB |
| Mapk1 | 21.398 | 183.58 | 3.0428 | 2.20E−16 | 1.55E−14 | TRUE | 2248.82 | 2650.83 | MINOCYCLINE; PURVALANOL |
| | | | | | | | | | B; MK-8353; CHIR-99021; |
| | | | | | | | | | RAVOXERTINIB; MK8353; |
| | | | | | | | | | ULIXERTINIB; ULIXERTINIB; |
| | | | | | | | | | RAVOXERTINIB; |
| | | | | | | | | | ULIXERTINIB |
| Cdk4 | 15.284 | 115.84 | 2.843 | 7.90E−12 | 3.83E−10 | TRUE | 2215.95 | 1711.23 | FASCAPLYSIN; PALBOCICLIB; |
| | | | | | | | | | AT-7519; ALVOCIDIB; |
| | | | | | | | | | RIVICICLIB; |
| | | | | | | | | | AZD-5438; ALVOCIDIB; |
| | | | | | | | | | RIVICICLIB; |
| | | | | | | | | | PALBOCICLIB; GIT28-1; |
| | | | | | | | | | RIBOCICLIB; ABEMACICLIB; |
| | | | | | | | | | PALBOCICLIB; ABEMACICLIB; |
| | | | | | | | | | AG-24322; RGB-286638; |
| | | | | | | | | | PALBOCICLIB; MILCICLIB; |
| | | | | | | | | | PALBOCICLIB; PHA-793887; |
| | | | | | | | | | LEROCICLIB; BMS- |
| | | | | | | | | | 387032; RIBOCICLIB; |
| | | | | | | | | | RONICICLIB; RIBOCICLIB; |
| | | | | | | | | | ABEMACICLIB; RIBOCICLIB; |
| | | | | | | | | | RIBOCICLIB; ABEMACICLIB; |
| | | | | | | | | | MILCICLIB; PALBOCICLIB; |
| | | | | | | | | | RGB-286638; RIBOCICLIB |
| | | | | | | | | | SUCCINATE; UCN-01; |
| | | | | | | | | | RG-547; VORUCICLIB; |
| | | | | | | | | | PALBOCICLIB; |
| | | | | | | | | | CHEMBL1956070; |
| | | | | | | | | | ALVOCIDIB; FOSTAMATINIB; |
| | | | | | | | | | TRILACICLIB; AT-7519; |
| | | | | | | | | | VORUCICLIB |
| Nfkb1 | 55.024 | 395.64 | 2.8237 | 2.20E−16 | 1.55E−14 | TRUE | 2498.15 | 2843.25 | PSEUDOEPHEDRINE |
| Ptpn6 | 10.19 | 73.63 | 2.7376 | 1.65E−08 | 5.16E−07 | TRUE | 5490.04 | 6461.60 | |
| Neu1 | 5.0948 | 38.287 | 2.6884 | 3.14E−06 | 6.71E−05 | TRUE | 1876.53 | 1760.32 | OSELTAMIVIR |
| Akt3 | 10.19 | 69.703 | 2.6596 | 1.21E−07 | 3.30E−06 | TRUE | 698.95 | 1391.80 | IPATASERTIB; OMIPALISIB; |
| | | | | | | | | | UPROSERTIB; GSK-690693; |
| | | | | | | | | | XL-418; TRICIRIBINE |
| | | | | | | | | | PHOSPHATE; A-443654; |
| | | | | | | | | | CAPIVASERTIB; AFURESERTIB; |
| | | | | | | | | | EVEROLIMUS; |
| | | | | | | | | | MSC-2363318A; GSK- |
| | | | | | | | | | 690693; MK-2206; AZD- |
| | | | | | | | | | 5363; MIRANSERTIB; MK- |
| | | | | | | | | | 2206; IPATASERTIB; LY- |
| | | | | | | | | | 2780301; IPATASERTIB; |
| | | | | | | | | | MK-2201; AFURESERTIB; |
| | | | | | | | | | UPROSERTIB; |
| Daglb | 26.493 | 166.89 | 2.6104 | 3.91E−11 | 1.76E−09 | TRUE | 877.90 | 6196.18 | ORLISTAT |
| Mknk1 | 47.891 | 294.52 | 2.5956 | 6.13E−15 | 3.81E−13 | TRUE | 1181.27 | 1695.29 | DORSOMORPHIN;; |
| | | | | | | | | | FOSTAMATINIB; TOMIVOSERTIB |
| Birc5 | 3.0569 | 22.58 | 2.5391 | 0.000137 | 0.002104 | TRUE | 1206.26 | 372.71 | |
| Ndufa13 | 34.645 | 202.24 | 2.5114 | 4.47E−11 | 1.99E−09 | TRUE | 1140.63 | 1422.21 | NV-128; METFORMIN |
| | | | | | | | | | HYDROCHLORIDE; ME- |
| | | | | | | | | | 344 |
| Hipk1 | 68.271 | 384.84 | 2.4777 | 3.14E−15 | 2.00E−13 | TRUE | 2203.29 | 2073.33 | STAUROSPORINE |
| Itgb7 | 38.721 | 218.93 | 2.4691 | 4.92E−11 | 2.18E−09 | TRUE | 627.95 | 1314.24 | VEDOLIZUMAB; NATALIZUMAB |
| Cd52 | 18.341 | 106.03 | 2.4682 | 1.37E−07 | 3.68E−06 | TRUE | 2921.43 | 4296.21 | ALEMTUZUMAB |
| Casp8 | 16.303 | 92.283 | 2.4306 | 7.97E−07 | 1.91E−05 | TRUE | 1279.99 | 939.87 | EMRICASAN; NIVOCASAN; |
| | | | | | | | | | EMRICASAN |
| Ccnd1 | 50.948 | 251.32 | 2.2801 | 1.24E−09 | 4.56E−08 | TRUE | 5561.25 | 3228.76 | ENCORAFENIB; PALBOCICLIB;; |
| | | | | | | | | | BRICICLIB |
| Lgmn | 52.986 | 260.16 | 2.2743 | 9.46E−10 | 3.57E−08 | TRUE | 8072.65 | 14865.05 | |
| Tbxas1 | 20.379 | 102.1 | 2.2698 | 4.86E−06 | 0.0001 | TRUE | 2346.82 | 2636.55 | OZAGREL; DAZOXIBEN; |
| | | | | | | | | | RIDOGREL; |
| Pak2 | 11.209 | 52.032 | 2.119 | 0.000732 | 0.009338 | TRUE | 1936.33 | 2588.47 | PF-03758309; |
| | | | | | | | | | FOSTAMATINIB; |
| | | | | | | | | | STAUROSPORINE |
| Usp14 | 14.265 | 62.831 | 2.064 | 0.000612 | 0.008015 | TRUE | 1054.17 | 1206.54 | |
| Gaa | 75.403 | 318.08 | 2.0622 | 1.61E−08 | 5.05E−07 | TRUE | 2202.35 | 2498.41 | MIGALASTAT; |
| | | | | | | | | | MIGLITOL; |
| | | | | | | | | | MIGLITOL; ACARBOSE |
| Tlr8 | 14.265 | 57.922 | 1.9485 | 0.002133 | 0.023407 | TRUE | 628.69 | 1921.71 | |
| Psmc3 | 21.398 | 84.429 | 1.9313 | 0.000681 | 0.008827 | TRUE | 2443.47 | 2079.28 | BORTEZOMIB; CARFILZOMIB; |
| | | | | | | | | | OPROZOMIB; |
| | | | | | | | | | CARFILZOMIB; BORTEZOMIB; |
| | | | | | | | | | IXAZOMIB CITRATE |
| Ulk2 | 8.1517 | 33.379 | 1.9094 | 0.010926 | 0.083007 | TRUE | 1369.91 | 1326.78 | FOSTAMATINIB |
| Cdk16 | 27.512 | 106.03 | 1.9083 | 0.000339 | 0.004769 | TRUE | 1317.89 | 885.40 | HESPERADIN; RONICICLIB; |
| | | | | | | | | | AT-7519; PHA-793887; |
| | | | | | | | | | FOSTAMATINIB; AZD-5438 |
| Pold1 | 34.645 | 132.53 | 1.9054 | 0.000126 | 0.00196 | TRUE | 1358.83 | 906.38 | GEMCITABINE |
| | | | | | | | | | HYDROCHLORIDE; |
| | | | | | | | | | CLOFARABINE; CYTARABINE; |
| | | | | | | | | | FLUDARABINE PHOSPHATE |
| Psma6 | 13.247 | 52.032 | 1.8962 | 0.004255 | 0.040919 | TRUE | 1560.53 | 1439.15 | CARFILZOMIB; CARFILZOMIB; |
| | | | | | | | | | OPROZOMIB; BORTEZOMIB; |
| | | | | | | | | | MARIZOMIB; IXAZOMIB |
| | | | | | | | | | CITRATE; BORTEZOMIB |
| Dpp7 | 21.398 | 81.484 | 1.8807 | 0.001215 | 0.014582 | TRUE | 583.28 | 1898.57 | |
| Parp1 | 23.436 | 85.41 | 1.8222 | 0.001693 | 0.019482 | TRUE | 2360.84 | 2381.84 | RUCAPARIB; NIRAPARIB; |
| | | | | | | | | | VELIPARIB; INIPARIB; |
| | | | | | | | | | VELIPARIB; INIPARIB; |
| | | | | | | | | | OLAPARIB; RUCAPARIB; |
| | | | | | | | | | OLAPARIB; OLAPARIB; |
| | | | | | | | | | NIRAPARIB; RUCAPARIB; |
| | | | | | | | | | VELIPARIB; VELIPARIB; |
| | | | | | | | | | TALAZOPARIB; OLAPARIB; |
| | | | | | | | | | NIRAPARIB; RUCAPARIB |
| | | | | | | | | | CAMSYLATE; TALAZOPARIB; |
| | | | | | | | | | TALAZOPARIB; RUCAPARIB; |
| | | | | | | | | | NIRAPARIB; NIRAPARIB; |
| | | | | | | | | | TALAZOPARIB TOSYLATE; |
| | | | | | | | | | VELIPARIB |
| Cat | 28.531 | 100.14 | 1.776 | 0.001455 | 0.017002 | TRUE | 1726.70 | 6709.64 | NABIXIMOLS; FOMEPIZOLE; |
| | | | | | | | | | CANNABIDIOL; |
| Cdk18 | 37.702 | 128.61 | 1.7437 | 0.000782 | 0.009912 | TRUE | 3513.22 | 7982.22 | AZD-5438; PHA-793887; |
| | | | | | | | | | RONICICLIB; AT-7519 |
| Aph1a | 124.31 | 416.25 | 1.7354 | 1.24E−06 | 2.83E−05 | TRUE | 1272.99 | 1371.97 | BEGACESTAT; SEMAGACESTAT; |
| | | | | | | | | | AVAGACESTAT; |
| Psmd11 | 17.322 | 59.886 | 1.7325 | 0.008773 | 0.071158 | TRUE | 1065.92 | 843.03 | BORTEZOMIB; IXAZOMIB |
| | | | | | | | | | CITRATE; CARFILZOMIB; |
| | | | | | | | | | CARFILZOMIB; BORTEZOMIB; |
| | | | | | | | | | OPROZOMIB |
| Rps6ka4 | 17.322 | 59.886 | 1.7325 | 0.008773 | 0.071158 | TRUE | 1416.01 | 1163.85 | HESPERADIN; |
| Cdk6 | 146.73 | 483.99 | 1.715 | 5.51E−07 | 1.36E−05 | TRUE | 1618.75 | 1166.23 | AT-7519; PHA-793887; |
| | | | | | | | | | ALVOCIDIB; RIBOCICLIB; |
| | | | | | | | | | AT-7519; VORUCICLIB; |
| | | | | | | | | | PALBOCICLIB; ABEMACICLIB; |
| | | | | | | | | | RIBOCICLIB SUCCINATE; |
| | | | | | | | | | RGB-286638; AZD-5438; |
| | | | | | | | | | TRILACICLIB; GIT28-1; |
| | | | | | | | | | UCN-01; ABEMACICLIB; |
| | | | | | | | | | RONICICLIB; LEROCICLIB |
| Vwf | 55.024 | 181.62 | 1.7047 | 0.000278 | 0.003995 | TRUE | 290.53 | 3256.48 | CAPLACIZUMAB |
| Map3k7 | 61.138 | 201.25 | 1.7026 | 0.000177 | 0.002653 | TRUE | 1039.51 | 900.08 | RGB-286638; |
| | | | | | | | | | CHEMBL1077979; |
| | | | | | | | | | CEP-11981 |
| Fos | 79.479 | 252.3 | 1.6542 | 0.000105 | 0.001675 | TRUE | 3523.76 | 7295.35 | NADROPARIN |
| Slc25a4 | 59.1 | 187.51 | 1.6492 | 0.000441 | 0.006051 | TRUE | 2832.06 | 2562.03 | ETIDRONIC ACID; |
| Capn2 | 83.555 | 262.12 | 1.6378 | 0.000107 | 0.001691 | TRUE | 1967.04 | 2182.56 | |
| Bptf | 59.1 | 182.6 | 1.6111 | 0.000728 | 0.009314 | TRUE | 1575.48 | 1916.94 | |
| Ptk2b | 62.157 | 190.46 | 1.6 | 0.00069 | 0.008927 | TRUE | 7057.36 | 6339.63 | FOSTAMATINIB; DEFACTINIB; |
| | | | | | | | | | BARICITINIB; HESPERADIN; |
| | | | | | | | | | PF-00562271; ALOISINE; |
| Kenn4 | 44.834 | 137.44 | 1.5948 | 0.002331 | 0.025275 | TRUE | 4974.27 | 5902.77 | CLOTRIMAZOLE; |
| | | | | | | | | | QUININE; HALOTHANE |
| Impa2 | 30.569 | 93.264 | 1.5782 | 0.007981 | 0.066651 | TRUE | 1038.35 | 391.97 | LITHIUM ION |
| Atp1b3 | 31.588 | 96.21 | 1.5768 | 0.007449 | 0.06379 | TRUE | 3991.41 | 4791.00 | ACETYLDIGITOXIN; |
| | | | | | | | | | DIGITOXIN; DESLANOSIDE; |
| | | | | | | | | | DIGOXIN |
| Fkbp1a | 77.441 | 232.67 | 1.5748 | 0.000395 | 0.005467 | TRUE | 1984.42 | 2488.37 | OLOCROLIMUS; TACROLIMUS; |
| | | | | | | | | | SIROLIMUS; SIROLIMUS; |
| | | | | | | | | | ZOTAROLIMUS; |
| | | | | | | | | | TACROLIMUS; TACROLIMUS; |
| | | | | | | | | | TEMSIROLIMUS; |
| | | | | | | | | | PIMECROLIMUS; EVEROLIMUS |
| Nampt | 37.702 | 113.88 | 1.5697 | 0.004993 | 0.046565 | TRUE | 1687.98 | 1854.49 | TEGLARINAD CHLORIDE |
| Fgfr1 | 35.664 | 107.01 | 1.5587 | 0.006394 | 0.056571 | TRUE | 1130.32 | 935.93 | PEMIGATINIB; ENMD-981693; |
| | | | | | | | | | INFIGRATINIB; ERDAFITINIB; |
| | | | | | | | | | INFIGRATINIB; |
| | | | | | | | | | DOVITINIB; SURUFATINIB; |
| | | | | | | | | | DEBIO-1347; LENVATINIB; |
| | | | | | | | | | ORANTINIB; XL-228; TG100- |
| | | | | | | | | | 801; PD-0166285; |
| | | | | | | | | | PAZOPANIB |
| | | | | | | | | | HYDROCHLORIDE; CEP- |
| | | | | | | | | | 11981; XL-999; |
| | | | | | | | | | PEXMETINIB; PAZOPANIB; |
| | | | | | | | | | PONATINIB; FOSTAMATINIB; |
| | | | | | | | | | NINTEDANIB; RG-1530; |
| | | | | | | | | | DERAZANTINIB; LUCITANIB; |
| | | | | | | | | | LY-2874455; DOVITINIB; |
| | | | | | | | | | LUCITANIB; SORAFENIB; |
| | | | | | | | | | DERAZANTINIB; MASITINIB; |
| | | | | | | | | | SUNITINIB; NINTEDANIB; |
| | | | | | | | | | BRIVANIB ALANINATE; |
| | | | | | | | | | BRIVANIB; |
| | | | | | | | | | ORANTINIB; DOVITINIB; |
| | | | | | | | | | PONATINIB; ROGARATINIB; |
| | | | | | | | | | RABEPRAZOLE SODIUM; |
| | | | | | | | | | NINTEDANIB ESYLATE; |
| | | | | | | | | | ROGARATINIB; |
| | | | | | | | | | INFIGRATINIB; NINTEDANIB; |
| | | | | | | | | | BRIVANIB; CP-459632; |
| | | | | | | | | | SORAFENIB; HESPERADIN; |
| | | | | | | | | | INFIGRATINIB; |
| | | | | | | | | | REGORAFENIB; FUTIBATINIB; |
| | | | | | | | | | LY-2874455; XL-228; |
| | | | | | | | | | ORANTINIB; SURUFATINIB; |
| | | | | | | | | | AZD-4547; NINTEDANIB; |
| | | | | | | | | | ERDAFITINIB; LUCITANIB; |
| | | | | | | | | | SU-014813; REGORAFENIB; |
| | | | | | | | | | PEMIGATINIB; MK-2461 |
| Nfkb2 | 43.815 | 128.61 | 1.5321 | 0.004689 | 0.044106 | TRUE | 1076.50 | 1206.66 | BORTEZOMIB; CHEMBL282093; |
| | | | | | | | | | GLYCYRRHIZIN; DONEPEZIL; |
| Eng | 91.707 | 266.05 | 1.5264 | 0.000383 | 0.005312 | TRUE | 613.72 | 1620.18 | CAROTUXIMAB |
| Cdk11b | 131.45 | 373.06 | 1.4979 | 0.000112 | 0.001767 | TRUE | 1176.42 | 1310.88 | RONICICLIB; AT- |
| | | | | | | | | | 7519; PHA-793887; AZD- |
| | | | | | | | | | 5438 |
| Egln2 | 23.436 | 67.739 | 1.4921 | 0.02624 | 0.144939 | TRUE | 988.91 | 1259.76 | ROXADUSTAT; DAPRODUSTAT; |
| Slc29a1 | 58.081 | 163.95 | 1.4813 | 0.00343 | 0.034394 | TRUE | 1120.07 | 863.46 | TROGLITAZONE; DRAFLAZINE; |
| | | | | | | | | | DIPYRIDAMOLE; DILAZEP; |
| | | | | | | | | | DIPYRIDAMOLE; |
| | | | | | | | | | FOSTAMATINIB; TICAGRELOR |
| Stat3 | 130.43 | 363.24 | 1.4706 | 0.000183 | 0.002735 | TRUE | 2706.93 | 2586.65 | |
| Rrm2 | 55.024 | 154.13 | 1.4694 | 0.004522 | 0.042881 | TRUE | 2222.14 | 753.07 | HYDROXYUREA; |
| | | | | | | | | | FLUDARABINE |
| | | | | | | | | | PHOSPHATE; GALLIUM |
| | | | | | | | | | NITRATE; CLADRIBINE; |
| | | | | | | | | | CLOFARABINE; HYDROXYUREA; |
| | | | | | | | | | GEMCITABINE; |
| | | | | | | | | | GEMCITABINE |
| | | | | | | | | | HYDROCHLORIDE; |
| | | | | | | | | | TEZACITABINE; FLUDARABINE; |
| | | | | | | | | | GALLIUM NITRATE; |
| | | | | | | | | | CLOFARABINE |
| Hipk2 | 81.517 | 226.78 | 1.4649 | 0.001367 | 0.016135 | TRUE | 1166.21 | 2805.06 | FOSTAMATINIB; |
| | 75.403 | 208.13 | 1.4527 | 0.002059 | 0.022819 | TRUE | 1794.75 | 1899.95 | LEVOMILNACIPRAN |
| | | | | | | | | | HYDROCHLORIDE; |
| | | | | | | | | | SERTRALINE |
| | | | | | | | | | HYDROCHLORIDE; |
| | | | | | | | | | LUMATEPERONE; |
| | | | | | | | | | MAZINDOL; |
| | | | | | | | | | TRAZODONE |
| | | | | | | | | | HYDROCHLORIDE; |
| | | | | | | | | | DESVENLAFAXINE |
| | | | | | | | | | SUCCINATE; |
| | | | | | | | | | PROTRIPTYLINE |
| Htt | | | | | | | | | HYDROCHLORIDE; |
| | | | | | | | | | PAROXETINE |
| | | | | | | | | | HYDROCHLORIDE; |
| | | | | | | | | | ESCITALOPRAMOXALATE; |
| | | | | | | | | | TEDATIOXETINE; |
| | | | | | | | | | AMITRIPTYLINE |
| | | | | | | | | | HYDROCHLORIDE; |
| | | | | | | | | | NEFAZODONE |
| | | | | | | | | | HYDROCHLORIDE; |
| | | | | | | | | | VENLAFAXINE |
| | | | | | | | | | HYDROCHLORIDE; |
| | | | | | | | | | METHAMPHETAMINE |
| | | | | | | | | | HYDROCHLORIDE; |
| | | | | | | | | | PAROXETINE |
| | | | | | | | | | MESYLATE; |
| | | | | | | | | | LIAFENSINE; |
| | | | | | | | | | AMOXAPINE; |
| | | | | | | | | | SIBUTRAMINE |
| | | | | | | | | | HYDROCHLORIDE; |
| | | | | | | | | | CLOMIPRAMINE |
| | | | | | | | | | HYDROCHLORIDE; |
| | | | | | | | | | DESVENLAFAXINE; |
| | | | | | | | | | IMIPRAMINE |
| | | | | | | | | | HYDROCHLORIDE; |
| | | | | | | | | | NORTRIPTYLINE |
| | | | | | | | | | HYDROCHLORIDE; |
| | | | | | | | | | FLUOXETINE |
| | | | | | | | | | HYDROCHLORIDE; |
| | | | | | | | | | VILAZODONE |
| | | | | | | | | | HYDROCHLORIDE; |
| | | | | | | | | | FLUVOXAMINE |
| | | | | | | | | | MALEATE; |
| | | | | | | | | | DASOTRALINE; |
| | | | | | | | | | VORTIOXETINE |
| | | | | | | | | | HYDROBROMIDE; |
| | | | | | | | | | DULOXETINE |
| | | | | | | | | | HYDROCHLORIDE; |
| | | | | | | | | | FAXELADOL; CITALOPRAM |
| | | | | | | | | | HYDROBROMIDE; |
| Ndufa10 | 47.891 | 132.53 | 1.4496 | 0.007865 | 0.065892 | TRUE | 1086.39 | 1138.64 | NV-128; ME- |
| | | | | | | | | | 344; METFORMIN |
| | | | | | | | | | HYDROCHLORIDE |
| Camk2d | 30.569 | 83.447 | 1.4195 | 0.025931 | 0.14365 | TRUE | 1397.94 | 1171.48 | FOSTAMATINIB |
| Eif4e | 65.214 | 173.77 | 1.4002 | 0.005515 | 0.050436 | TRUE | 1146.60 | 910.83 | |
| Eef2 | 52.986 | 140.39 | 1.389 | 0.010338 | 0.080156 | TRUE | 45293.17 | 50097.95 | DENILEUKIN |
| | | | | | | | | | DIFTITOX; |
| | | | | | | | | | MOXETUMOMAB |
| | | | | | | | | | PASUDOTOX; |
| | | | | | | | | | ESKETAMINE |
| Pik1 | 84.574 | 222.85 | 1.3873 | 0.00296 | 0.030509 | TRUE | 1112.02 | 250.67 | GSK-461364; BI- |
| | | | | | | | | | 2536; GSK-461364; BI- |
| | | | | | | | | | 2536; TAK-960; |
| | | | | | | | | | FOSTAMATINIB; |
| | | | | | | | | | CAFUSERTIB; VOLASERTIB; |
| | | | | | | | | | NMS-1286937; GSK- |
| | | | | | | | | | 579289A; MK-1496; |
| | | | | | | | | | ONVANSERTIB; VOLASERTIB; |
| | | | | | | | | | GSK-461364; HMN-214; |
| | | | | | | | | | GSK-461364; BI-2536; |
| | | | | | | | | | VOLASERTIB; VOLASERTIB; |
| | | | | | | | | | NMS-1286937; |
| Mapk14 | 104.95 | 269.98 | 1.3547 | 0.002154 | 0.023606 | TRUE | 3001.35 | 2997.59 | AMG-548; SC-80036; |
| | | | | | | | | | SEMAPIMOD; RALIMETINIB; |
| | | | | | | | | | R-1487; RWJ-67657; |
| | | | | | | | | | AZD-6703; TALMAPIMOD; |
| | | | | | | | | | DORAMAPIMOD; PG-760564; |
| | | | | | | | | | TA-5493; SCIO-323; |
| | | | | | | | | | LOSMAPIMOD; PH- |
| | | | | | | | | | 797804; RALIMETINIB; |
| | | | | | | | | | DILMAPIMOD; RO- |
| | | | | | | | | | 3201195; ACUMAPIMOD; |
| | | | | | | | | | TAK-715; VX-702; FX- |
| | | | | | | | | | 005; PEXMETINIB; |
| | | | | | | | | | DORAMAPIMOD; GSK- |
| | | | | | | | | | 610677; NEFLAMAPIMOD; |
| | | | | | | | | | CHEMBL1236539; BMS- |
| | | | | | | | | | 582949; PS-516895; AVE- |
| | | | | | | | | | 9940; BMS-582949; |
| | | | | | | | | | DILMAPIMOD; SD- |
| | | | | | | | | | 0006; LY-3007113; |
| | | | | | | | | | PAMAPIMOD; AZD- |
| | | | | | | | | | 7624; SB-85635; LEO- |
| | | | | | | | | | 15520; PAMAPIMOD; |
| | | | | | | | | | TALMAPIMOD; PH- |
| | | | | | | | | | 797804; ARRY- |
| | | | | | | | | | 797; ACUMAPIMOD; PF- |
| | | | | | | | | | 03715455; CHEMBL1951415; |
| | | | | | | | | | FOSTAMATINIB; LOSMAPIMOD; |
| | | | | | | | | | PF-03715455; KC-706 |
| Akr1b10 | 71.327 | 180.64 | 1.3285 | 0.008521 | 0.069738 | TRUE | 639.75 | 1009.55 | EXISULIND; |
| | | | | | | | | | FIDARESTAT; |
| | | | | | | | | | SULINDAC |
| Kif11 | 50.948 | 128.61 | 1.319 | 0.019534 | 0.120946 | TRUE | 1429.92 | 534.59 | AZD-4877; LITRONESIB |
| Sod1 | 57.062 | 142.35 | 1.3039 | 0.017352 | 0.112968 | TRUE | 3051.35 | 4875.99 | NABIXIMOLS; |
| | | | | | | | | | CANNABIDIOL |
| Anpep | 51.967 | 129.59 | 1.3019 | 0.021279 | 0.12813 | TRUE | 1672.97 | 21248.16 | TOSEDOSTAT; |
| | | | | | | | | | ICATIBANT |
| Rptor | 142.65 | 346.55 | 1.2746 | 0.00205 | 0.022737 | TRUE | 1105.26 | 1259.49 | AZD-8055; |
| | | | | | | | | | SAPANISERTIB; OSI-027 |
| Pik3cd | 84.574 | 198.31 | 1.2198 | 0.014662 | 0.101152 | TRUE | 2453.02 | 4629.07 | TASELISIB; APITOLISIB; |
| | | | | | | | | | IDELALISIB; SERABELISIB; |
| | | | | | | | | | BIMIRALISIB; |
| | | | | | | | | | CHEMBL1086377; PWT- |
| | | | | | | | | | 33587; BUPARLISIB; |
| | | | | | | | | | PICTILISIB; GEDATOLISIB; |
| | | | | | | | | | GSK-2269557; |
| | | | | | | | | | PARSACLISIB; AZD- |
| | | | | | | | | | 6482; FIMEPINOSTAT; |
| | | | | | | | | | DUVELISIB; COPANLISIB; |
| | | | | | | | | | APITOLISIB; RECILISIB; |
| | | | | | | | | | PANULISIB; INCB- |
| | | | | | | | | | 40093; DACTOLISIB; |
| | | | | | | | | | SONOLISIB; FIMEPINOSTAT; |
| | | | | | | | | | DS-7423; ME-401; |
| | | | | | | | | | TENALISIB; PICTILISIB; |
| | | | | | | | | | DACTOLISIB; VOXTALISIB; |
| | | | | | | | | | COPANLISIB; AZD- |
| | | | | | | | | | 6482; PF-04691502; |
| | | | | | | | | | ALPELISIB; SF- |
| | | | | | | | | | 1126; OMIPALISIB; |
| | | | | | | | | | UMBRALISIB; GSK-2636771; |
| | | | | | | | | | GEDATOLISIB; NEMIRALISIB; |
| | | | | | | | | | TG100-115; |
| | | | | | | | | | SONOLISIB; AMG- |
| | | | | | | | | | 319; ZSTK-474; |
| | | | | | | | | | DUVELISIB; TG100-115; |
| | | | | | | | | | UMBRALISIB |
| | | | | | | | | | TOSYLATE; IDELALISIB; |
| | | | | | | | | | AZD-8186; CAL-263; |
| | | | | | | | | | COPANLISIB; LENIOLISIB; |
| | | | | | | | | | APITOLISIB; PILARALISIB; |
| | | | | | | | | | AMG-319; PUQUITINIB; |
| | | | | | | | | | PANULISIB; CAFFEINE; |
| | | | | | | | | | AZD-8835; PF-04691502; |
| | | | | | | | | | DEZAPELISIB; PA- |
| | | | | | | | | | 799; PI-103; |
| | | | | | | | | | VOXTALISIB; GDC- |
| | | | | | | | | | 0077; IDELALISIB; |
| | | | | | | | | | SELETALISIB; |
| | | | | | | | | | SAMOTOLISIB; ACALISIB; |
| | | | | | | | | | INFIGRATINIB; RP- |
| | | | | | | | | | 6530; GEDATOLISIB; |
| | | | | | | | | | SAMOTOLISIB; PICTILISIB; |
| | | | | | | | | | GSK-1059615; |
| | | | | | | | | | WORTMANNIN; AZD- |
| | | | | | | | | | 8186; COPANLISIB; |
| | | | | | | | | | QUERCETIN; PUQUITINIB; |
| | | | | | | | | | SAPANISERTIB; COPANLISIB; |
| | | | | | | | | | FOSTAMATINIB; ALPELISIB; |
| | | | | | | | | | PILARALISIB; |
| | | | | | | | | | RG-7666; WX-037; |
| | | | | | | | | | IDELALISIB; VS-5584; |
| | | | | | | | | | PILARALISIB; OMIPALISIB; |
| | | | | | | | | | TASELISIB; ACALISIB; |
| | | | | | | | | | ALPELISIB; IDELALISIB; |
| | | | | | | | | | BUPARLISIB; SONOLISIB; |
| | | | | | | | | | SAR-260301; |
| | | | | | | | | | DACTOLISIB; DUVELISIB; |
| | | | | | | | | | BGT-226; INCB- |
| | | | | | | | | | 40093; VS-5584; |
| | | | | | | | | | OMIPALISIB; PI-103; |
| | | | | | | | | | DACTOLISIB; |
| Slc12a7 | 99.858 | 230.71 | 1.2 | 0.011983 | 0.088607 | TRUE | 1294.77 | 1485.18 | |
| Slc44a1 | 69.29 | 157.08 | 1.1692 | 0.031657 | 0.163545 | TRUE | 1468.84 | 2239.94 | HEMICHOLINIUM-3 |
| Prkcb | 82.536 | 185.55 | 1.1591 | 0.024771 | 0.140449 | TRUE | 1453.74 | 3078.28 | SOTRASTAURIN; GSK- |
| | | | | | | | | | 690693; ENZASTAURIN; |
| | | | | | | | | | MIDOSTAURIN; |
| | | | | | | | | | ENZASTAURIN; ENZASTAURIN; |
| | | | | | | | | | RUBOXISTAURIN; ELLAGIC |
| | | | | | | | | | ACID; ENZASTAURIN; |
| | | | | | | | | | CEP-2563; QUERCETIN; |
| | | | | | | | | | RUBOXISTAURIN; |
| | | | | | | | | | CHEMBL1236539; |
| | | | | | | | | | SOTRASTAURIN; |
| | | | | | | | | | UCN-01; BALANOL |
| Cdk13 | 98.839 | 218.93 | 1.1393 | 0.020313 | 0.124359 | TRUE | 1106.29 | 1221.26 | RONICICLIB; AZD- |
| | | | | | | | | | 5438; AT-7519; PHA- |
| | | | | | | | | | 793887 |
| Notch4 | 244.55 | 537.01 | 1.1316 | 0.002128 | 0.023394 | TRUE | 3194.29 | 47.20 | RG-4733; |
| | | | | | | | | | NIROGACESTAT |
| Psma5 | 85.593 | 187.51 | 1.1223 | 0.030235 | 0.158502 | TRUE | 1112.94 | 1045.08 | CARFILZOMIB; CARFILZOMIB; |
| | | | | | | | | | BORTEZOMIB; BORTEZOMIB; |
| | | | | | | | | | OPROZOMIB; IXAZOMIB |
| | | | | | | | | | CITRATE; MARIZOMIB |
| Fnta | 90.688 | 197.33 | 1.1131 | 0.029136 | 0.153854 | TRUE | 984.83 | 1210.41 | LONAFARNIB; TIPIFAR |
| | | | | | | | | | NIB; TIPIFARNIB |
| Pi4ka | 105.97 | 229.72 | 1.1089 | 0.022491 | 0.132581 | TRUE | 1278.10 | 1820.72 | WORTMANNIN; |
| Pkm | 118.2 | 241.51 | 1.0246 | 0.034681 | 0.172926 | TRUE | 66120.62 | 38895.17 | |
| Hipk3 | 138.58 | 280.77 | 1.0135 | 0.028564 | 0.152177 | TRUE | 1687.51 | 1764.01 | FOSTAMATINIB; |
| | | | | | | | | | SILMITASERTIB |
| Tubb6 | 141.64 | 286.67 | 1.0121 | 0.027763 | 0.150009 | TRUE | 4341.03 | 1466.10 | BRENTUXIMAB VEDOTIN; |
| | | | | | | | | | FOSBRETABULIN |
| | | | | | | | | | DISODIUM; |
| | | | | | | | | | LEXIBULIN; IXABEPILONE; |
| | | | | | | | | | VINORELBINE |
| | | | | | | | | | TARTRATE; VINBLASTINE |
| | | | | | | | | | SULFATE; |
| | | | | | | | | | INDIBULIN; CROLIBULIN; |
| | | | | | | | | | TRASTUZUMABEMTANSINE; |
| | | | | | | | | | VINFLUNINE; |
| | | | | | | | | | CABAZITAXEL; VINCRISTINE |
| | | | | | | | | | SULFATE; ERIBULIN |
| | | | | | | | | | MESYLATE; DOCETAXEL; |
| | | | | | | | | | PLINABULIN; COLCHICINE; |
| | | | | | | | | | PACLITAXEL |
| Ctso | 168.13 | 336.73 | 0.99776 | 0.022619 | 0.132581 | TRUE | 572.54 | 1055.76 | ODANACATIB |

Collectively, these data show that the organotypic TME model described herein can be leveraged for high-throughput target discovery to reveal novel and druggable mediators of the immunosuppressive phenotype in TME macrophages.

### Example 4

### Single-Cell RNA-Seq of Macrophage Education Time-Course Reveals Early Activation of Cell-Cycle Triggered by Transient Activation of Type-I Interferons

The oTME offers a unique opportunity to study the dynamics of macrophage education. The inventors therefore performed time-course single-cell RNA sequencing (scRNA-seq), comparing macrophages educated in the oTME at two (“Early”) or ten days (“Late”) time-points, vs. control macrophages maintained with M-CSF for the same intervals (“M-CSF-treated”) (FIG. 3A). The inventors profiled 19,280 single-cell transcriptomes (10× Genomics; STAR Methods) and clustered the cells into time-point and four major cell lineages (FIG. 3B) — stromal-like cells (α-smooth muscle actin positive; Acta2, Cd24a-, ECM-related genes, Crip2), tumor epithelial cells (Epcam Cd24a+), mesenchymal cells (Epcam-, Cd24a, Krt7), and macrophages (Cd68) (FIG. 4A).

To evaluate the phenotypic fidelity of macrophages in this model to human breast cancer macrophages, the inventors first generated an education signature by comparing differential expression between control and late educated macrophages, and projected this signature (FIG. 4B; day 10) onto a recently published scRNA-seq dataset of human breast cancer macrophages (Azizi et al., “Single-Cell Map of Diverse Immune Phenotypes in the Breast Tumor Microenvironment,” Cell 174(36):1293-1308 (2018), which is hereby incorporated by reference in its entirety) (STAR Methods). Notably, the ex-vivo signatures were able to distinguish intratumoral macrophages from other myeloid cells, but also recapitulated the transcriptional signatures and activation markers of the three subpopulations of macrophages with wound healing phenotype (MRC1, LGALS3, MARCO, APOE, FN1, MMP14), as reported by Azizi et al., “Single-Cell Map of Diverse Immune Phenotypes in the Breast Tumor Microenvironment,” Cell 174(36):1293-1308 (2018), which is hereby incorporated by reference in its entirety (FIG. 4C; Human TAMs clusters 25,23,28). These transcriptional similarities between the oTME and human macrophages further indicate the system's ability to accurately model of the phenotypic alterations in breast cancer macrophages.

To study the dynamics of transcriptional alterations in macrophages during TME education, the inventors reconstructed an “education trajectory” by overlaying controls, early, and late single-cell transcriptomes onto a pseudo-temporal projection (FIG. 3C; STAR Methods). The education process was marked by down-regulation of genes that mediate immunostimulatory responses, including GM-CSF/IL-5 and TNF receptors signaling (Csf2ra, Tnfrsflb, Itgax, Mmp12) (Kratochvill et al., “TNF Counterbalances the Emergence of M2 Tumor Macrophages,” Cell Rep. 12:1902-1914 (2015) and Quail et al., “Obesity Alters the Lung Myeloid Cell Landscape to Enhance Breast Cancer Metastasis Through IL5 and GM-CSF,” Nat. Cell Biol. 19:974-987 (2017), which are hereby incorporated by reference in their entirety) , intracellular iron storage (Fthl), and antigen presentation (H2-Aa, Cd74) (FIG. 3D; M-CSF-Treated). Interestingly, these changes were coupled with elevation of apoptotic cell clearance (Fcgr1, Fcr1s, Ax1, Tgm2), lipid oxidative stress (Tlr2, Cd36, Lp1) (Kim et al., “Carcinoma-Produced Factors Activate Myeloid Cells Through TLR2 to Stimulate Metastasis,” Nature 457:102-106 (2009); Motwani et al., “DNA Sensing by the cGAS-STING Pathway in Health and Disease,” Nat. Rev. Genet. 20:657-674 (2019); and West et al., “Oxidative Stress Induces Angiogenesis by Activating TLR2 With Novel Endogenous Ligands,” Nature 467:972-976 (2010), which are hereby incorporated by reference in their entirety), and a transient pro-inflammatory activation of type-I interferon response (Isg15, Rtp4, Ifit1) mediated by Irf7 (FIG. 3D-E;) (Bidwell et al., “Silencing of Irf7 Pathways in Breast Cancer Cells Promotes Bone Metastasis Through Immune Escape,” Nat. Med. 18:1224-1231 (2012); Honda and Taniguchi, “IRFs: Master Regulators of Signalling by Toll-Like Receptors and Cytosolic Pattern-Recognition Receptors,” Nat. Rev. Immunol. 6:644-658 (2006); and Honda et al., “IRF-7 is the Master Regulator of Type-I Interferon-Dependent Immune Responses,” Nature 434:772-777 (2005), which are hereby incorporated by reference in their entirety) that coincided with cell-cycle activation (Mki67, Top2a) (FIG. 3D and FIG. 3E; Early). As the education process continued, macrophages transitioned from pro-inflammatory into anti-inflammatory phenotype, associated with matrix remodeling (Mmp14, Fn1, Ecm1), ER stress (Xbpl), blood coagulation (Pf4, F13a1), pro-angiogenic (Egln3, Vegfa, Eno1) and immune modulation (Arg1, Thbs1, Mrc1, Apoe, Ptgs1) gene signatures (FIG. 3D and FIG. 3E; Late).

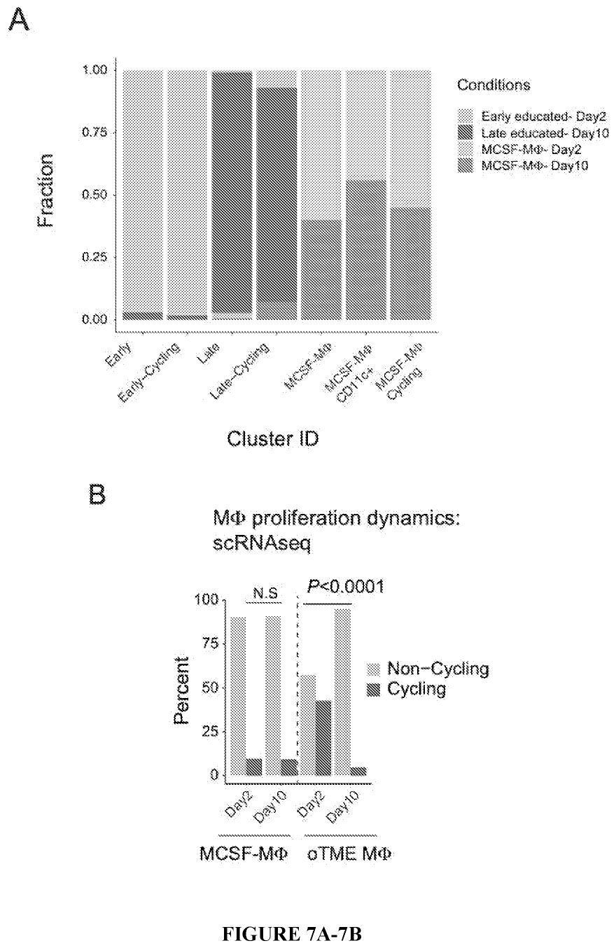

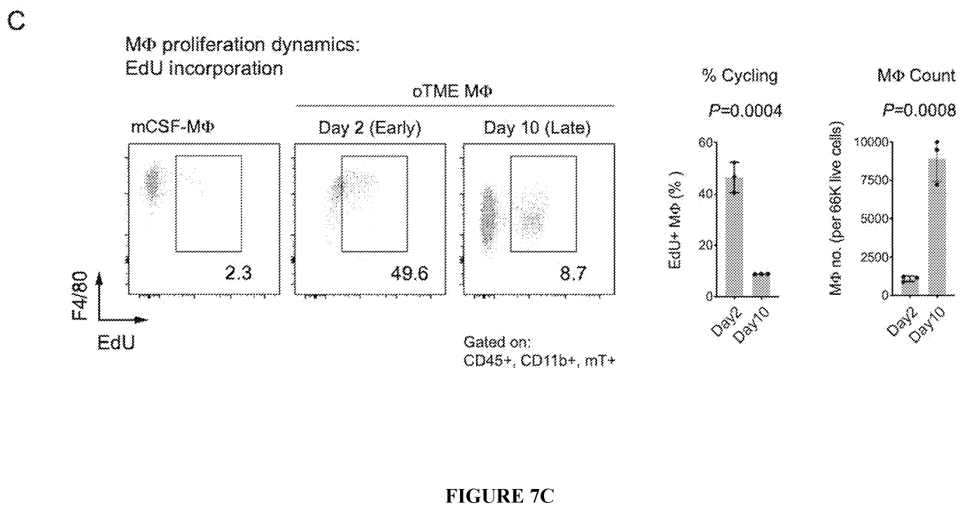

To investigate the cell-cycle activation mechanisms in oTME macrophages, the inventors further annotated single-cell macrophage transcriptomes (from FIG. 3C) based on their education conditions (Control, Early, or Late educated) and cell-cycle states (cycling and non-cycling) (FIG. 7A; STAR Methods). In M-CSF-treated control macrophages, three distinct clusters were identified by unsupervised clustering according to gene expression signatures (FIG. 3F); cycling (Mki67; 9.6%), non-cycling pro-inflammatory Cd11c+(43.2%; Itgax, H2-Aa, Csf2ra), and non-cycling (47.2%) macrophages. Similarly, two clusters of oTME macrophages were identified in each of the early and late time points; cycling (Day2: 45.4%; Day10: 5.1%) and non-cycling cells (Day2: 54.6%; Day10: 94.9%). In the M-CSF-treated macrophages, no significant changes in the fraction of cycling cells were observed between early (Day 2; 9.7%) and late (Day 10; 9.1%) time-points (P=0.6138, two-proportions Z-test; FIG. 7B). In contrast, after two days of oTME education, while macrophages still lacked canonical anti-inflammatory/would healing markers (Arg1, Fn1, F13a 1; FIG. 3F), almost half of them (42.8%) engaged the cell-cycle and uniquely marked by Tmem173, an indication for STING/type-I interferons activation in cycling cells (Motwani et al., “DNA Sensing by the cGAS-STING Pathway in Health and Disease,” Nat. Rev. Genet. 20:657-674 (2019), which is hereby incorporated by reference in its entirety). After ten days of education, most macrophages turned quiescent and anti-inflammatory while a small subset remained proliferative (from 42.8% to 4.7%, P<2.2×10-16, two-proportions Z-test). Interestingly, unlike the quiescent cells, this proliferative subset retained the expression of pro-inflammatory mediators Csf2ra and Bgn, a matrix component act as a signaling molecule through TLR4/2 pathway (FIG. 3F) (Schaefer et al., “The Matrix Component Biglycan is Proinflammatory and Signals Through Toll-Like Receptors 4 and 2 in Macrophages,” J. Clin. Invest. 115:2223-2233 (2005); Xue et al., “Transcriptome-Based Network Analysis Reveals a Spectrum Model of Human Macrophage Activation,” Immunity 40:274-288 (2014), which are hereby incorporated by reference in their entirety). These dynamics were validated orthogonally with EdU labeling (FIG. 7C) and suggest that macrophage proliferation in tumors initiates at early stages and requires a sustained inflammatory activation in a specific subpopulation.

A non-cell cycle gene, Ly6A (Stem Cell Antigen-1; Sca-1), was associated with the cycling subset of oTME macrophages (FIG. 3F). Interestingly, Ly6A is known to be upregulated in response to acute activation of type-I interferons (via IFNAR1 signaling) and mediate a robust reactivation of cell cycle in dormant hematopoietic stem cells (HSCs) (Essers et al., “IFNalpha Activates Dormant Haematopoietic Stem Cells in Vivo,” Nature 458:904-908 (2009); Ito et al., “Hematopoietic Stem Cell and Progenitor Defects in Sca-1/Ly-6A-null Mice,” Blood 101:517-523 (2003); Walter et al., “Exit From Dormancy Provokes DNA-Damage-Induced Attrition in Haematopoietic Stem Cells,” Nature 520:549-552 (2015), which are hereby incorporated by reference in their entirety) . Next, whether early activation of type-I interferons and onset of cell cycle in macrophages (FIG. 3G) were functionally related responses were examined. EdU incorporation in macrophages was measured following 48 hrs education in the presence of Tyk2 inhibitors that block the type-I interferon response (Burke et al., “Autoimmune Pathways in Mice and Humans are Blocked by Pharmacological Stabilization of the TYK2 Pseudokinase Domain,” Sci. Transl. Med. 11 (2019); Prchal-Murphy et al., “TYK2 Kinase Activity is Required for Functional Type I Interferon Responses In Vivo,” PLoS One 7:e39141 (2012), which are hereby incorporated by reference in their entirety). Since Tyk2 inhibition significantly decreased EdU incorporation in oTME macrophages without affecting their Arg1+ expression (FIG. 3H), it was concluded that early activation of type-I interferons was required to induce macrophage proliferation marked by upregulation of Ly6A.

### Example 5

### Macrophage Proliferation is Facilitated Through Cell Contact wWith Stromal Cells

The induction of Ly6A in macrophages from mammary tumors and oTME

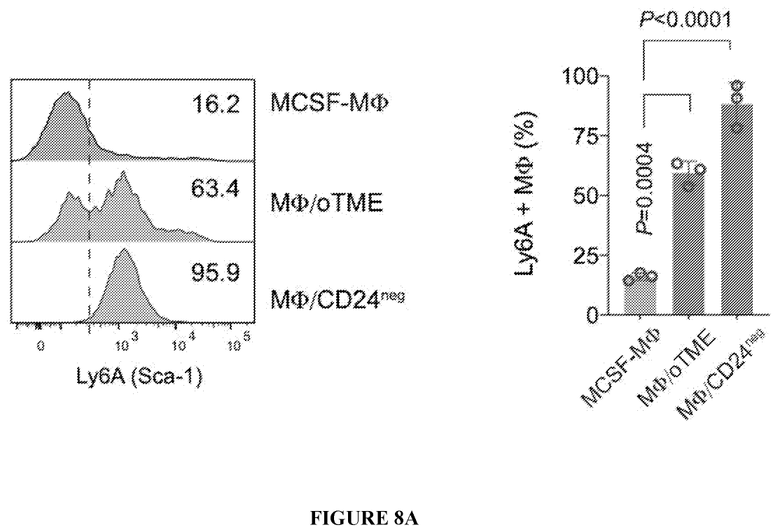

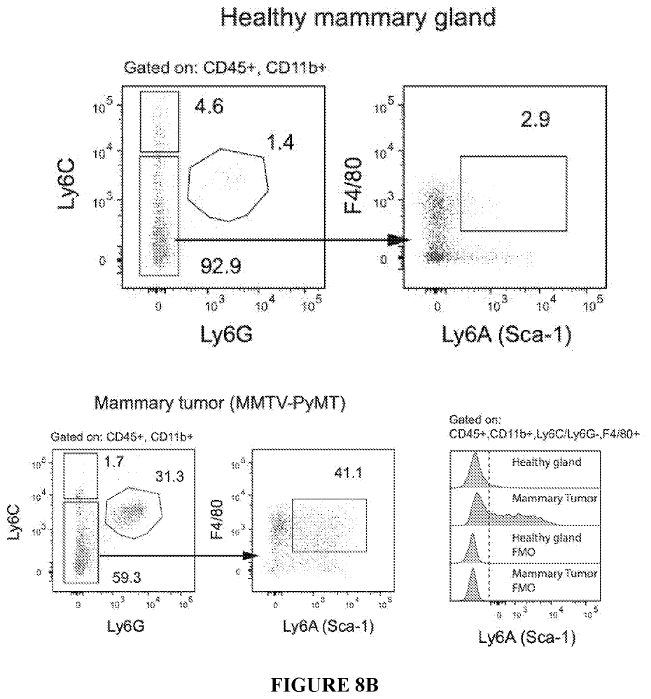

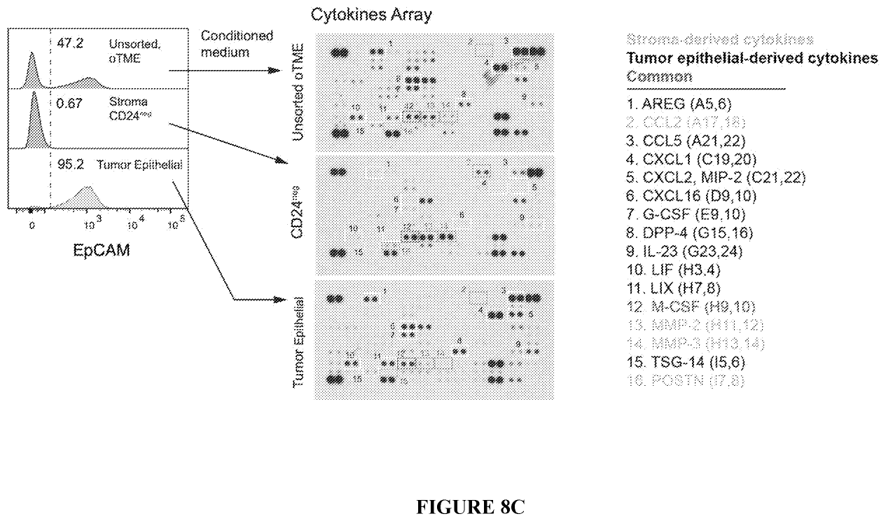

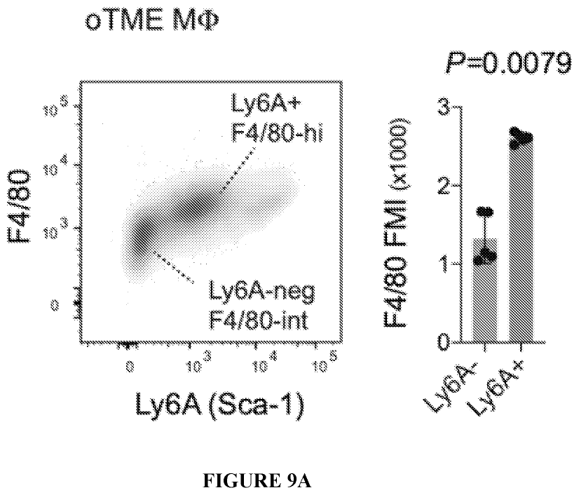

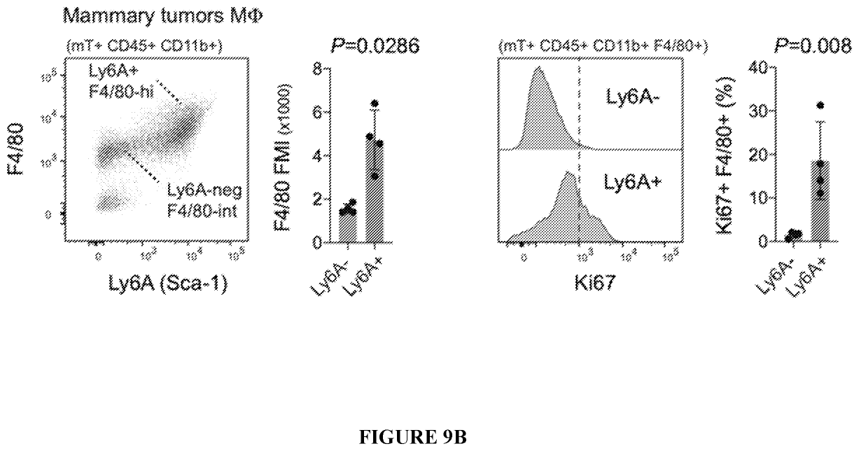

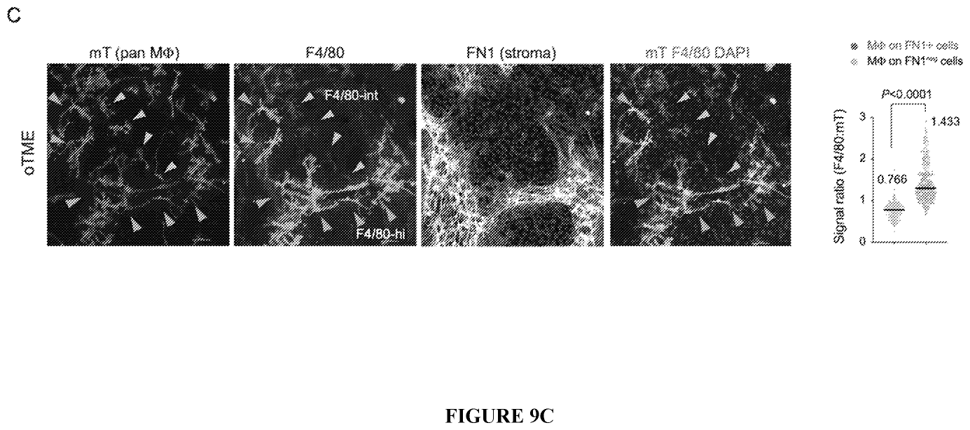

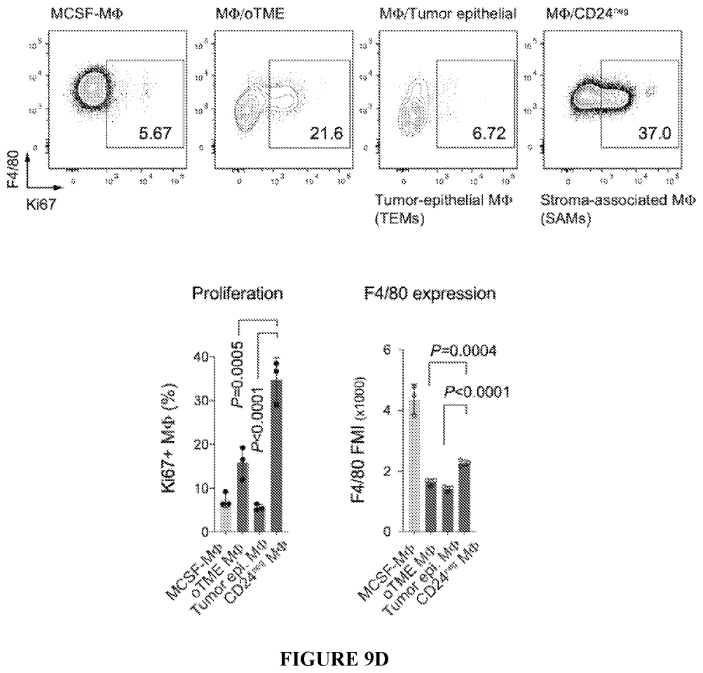

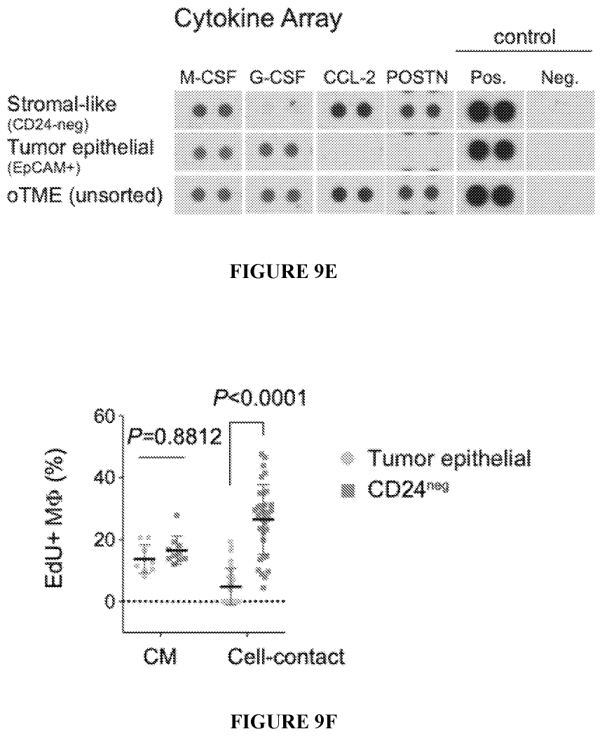

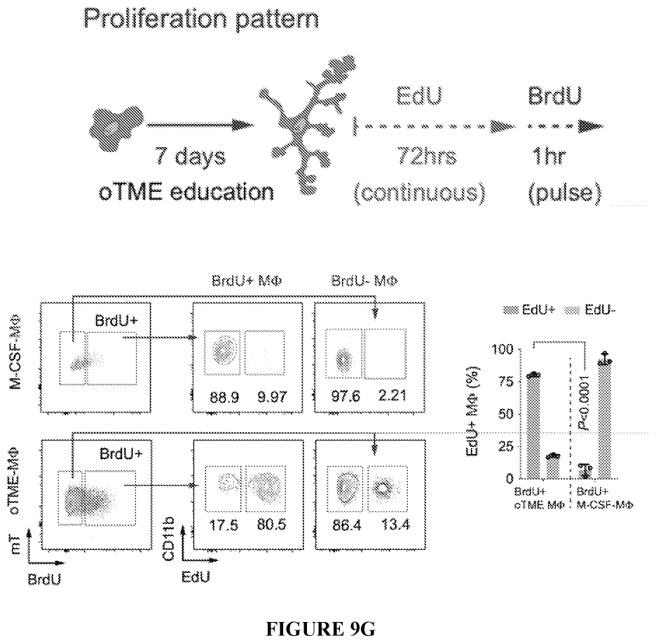

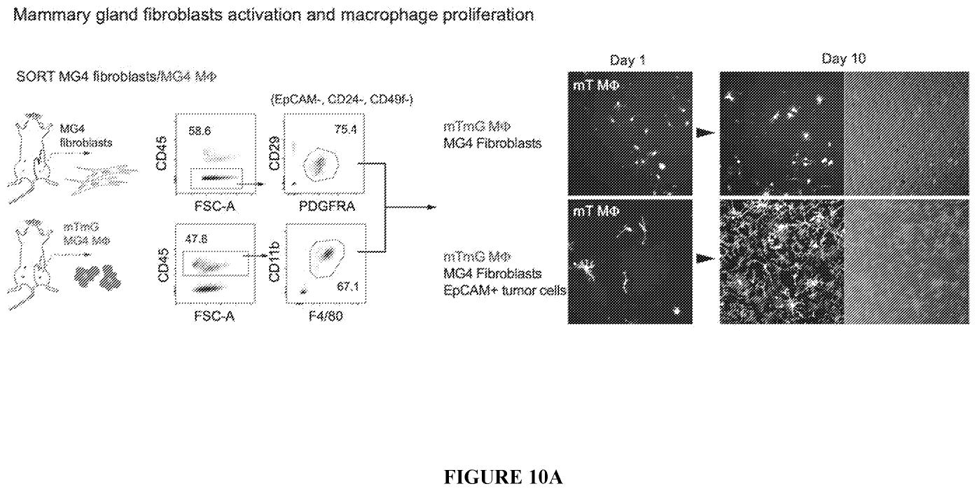

cultures, particularly when cultured with CD24^{neg} cells was confirmed (FIG. 8A and FIG. 8B). Notably, when oTME macrophages were stained with the pan macrophage marker F4/80 and Ly6A (Sca-1), it was observed that Ly6A consistently marked a subpopulation of F4/80^{high} but was absent/low in F4/80^{int} cells (P=0.0079, 2-tailed unpaired Student's t-test) (FIG. 9A). The association between Ly6A with F4/80^{high} macrophages in mammary tumors validated (FIG. 9B, left), and it was observed that the cycling fraction (Ki67+) was predominantly Ly6A^{high} (FIG. 9B, right) (P=0.008, 2-tailed unpaired Student's t-test).

To determine if there are spatio-functional differences between the F4/80^{high} and F4/80^{int} populations, the inventors immunostained oTME mTmG macrophages for F4/80 along with Fibronectin (FN1) to highlight stromal areas (FIG. 9C). Macrophages (tdTomato+) exhibited two populations with intermediate (F4/80^{int}) or high (F4/80^{high}) F4/80 staining, recapitulating the flow cytometry data. Interestingly, whereas the F4/80^{int} macrophages predominantly localized inside tumor epithelial nests (hereafter referred to tumor-epithelial macrophages—TEMs), F4/80^{high} macrophages were exclusively localized in FN1+ stromal areas (hereafter referred to as stroma-associated macrophages —SAMs). The expression differences of F4/80 as a function of physical localization were further quantified with a higher signal ratios of F4/80 over mT in SAMs vs. TEMs (1.43 vs. 0.76; P<0.0001, Mann-Whitney test).

Given the close association between F4/80^{high} Ly6A^{high} and macrophage proliferation in mammary tumors, these findings suggest that SAMs are predominantly responsible for replenishing macrophages in the TME. To identify the cellular components in the oTME that drive macrophage proliferation, the inventors plated quiescent macrophages (Ki67-) together with FACS-sorted tumor epithelial cells (EpCAM+), stromal-like cells (CD24^{neg}PDGFRA+), oTME cells, or M-CSF alone as control. After 10 days, macrophage proliferation was significantly enhanced by CD24^{neg}PDGFRA+ cells compared with tumor epithelial cells or M-CSF alone (FIG. 9D). Moreover, the Ki67+ oTME macrophages were associated with F4/80^{high} (consistent with SAMs) while the Ki67^{neg} were associated with F4/80^{int} (consistent with TEMs). Similar results were observed in macrophages from 3D cultures of partially-digested MMTV-PyMT primary tumors.

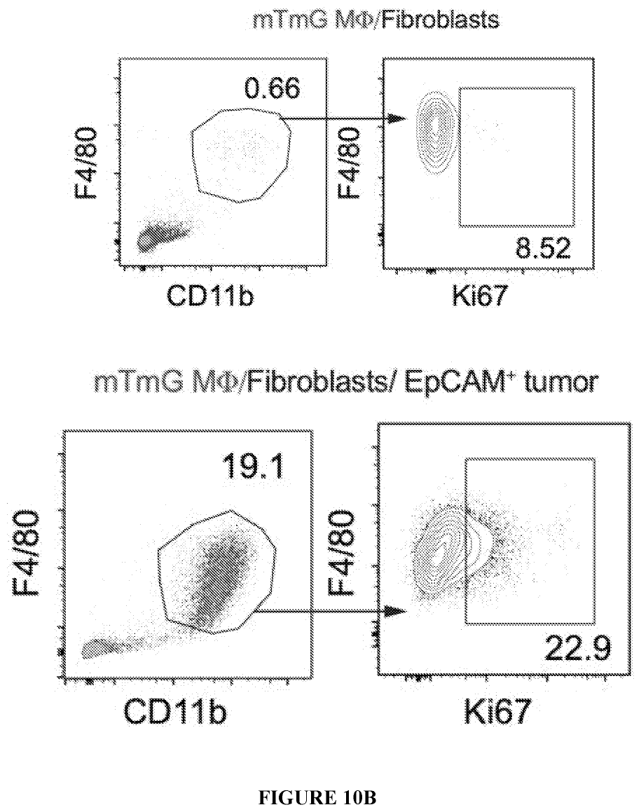

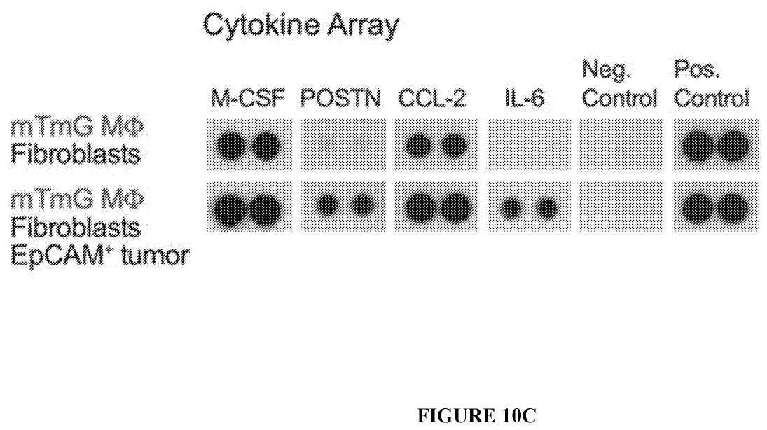

Distinct cytokine production was observed between purified stromal-like and EpCAM+ epithelial cultures, including CCL-2 and POSTN specific production from stromal-like cells, while G-CSF, IL-23, and CCL-5 were derived specifically from tumor epithelial cells (FIG. 9E and FIG. 8C). However, M-CSF, the main cytokine known to drive macrophage proliferation in mammary tissues (Pollard and Hennighausen, “Colony Stimulating Factor 1 is Required for Mammary Gland Development During Pregnancy,” Proc. Natl. Acad. Sci. U.S.A. 91:9312-9316 (1994), which is hereby incorporated by reference in its entirety), was similarly detected across the culture conditions, indicating that M-CSF availability is not sufficient to license enhanced proliferation in macrophages. Critically, the inventors validated similar responses with resting fibroblasts and macrophages that were purified from healthy mammary glands (FIG. 10A through FIG. 10C). An enhanced proliferation of mammary gland macrophages (mT+) was observed when fibroblasts were activated by the presence of tumor epithelial cells (activation cytokines; POSTN+, IL-6+), while M-CSF was evenly detected in resting fibroblasts/macrophage cultures (POSTN^{neg},IL-6^{neg}).

Next, the inventors sought to determine whether macrophage proliferation is licensed by cell contact. The inventors compared EdU incorporation in macrophages that were either allowed physical contact with tumor epithelial, CD24^{neg}PDGFRA+ cells, or treated individually with their conditioned media. EdU incorporation rates were similar in response to either CM, but when physical contact was allowed, SAMs displayed significantly higher EdU incorporation (FIG. 9F). These results indicate that physical interactions between macrophages and fibroblasts promote enhanced proliferation in macrophages. Whether proliferation of educated macrophages occurs in a specific subpopulation (as suggested by the scRNA-seq data; FIG. 3F) was examined. To track proliferation history, a dual pulse-chase labeling strategy with EdU and BrdU was utilized, as it enables scoring the same cells for cell cycle at two different time points. Control (M-CSF-treated) and 7-days educated macrophages were labeled with EdU for 72 hrs followed by lhr pulse of BrdU (FIG. 9G, left). In agreement with leaving the cell cycle upon differentiation (Klappacher et al., “An Induced Ets Epressor Complex Regulates Growth Arrest During Terminal Macrophage Differentiation,” Cell 109:169-180 (2002), which is hereby incorporated by reference in its entirety), the cycling subset (BrdU+) in M-CSF-treated macrophage displayed low proliferative history (EdU+; 6.9%±4.3% mean±SD). However, in oTME macrophages, the BrdU+cycling subset displayed a substantial proliferative history (EdU+; 80.2%±1.1%), while non-proliferating BrdU^{neg} cells showed minimal proliferation history (FIG. 9G, right). The continuous proliferation in a specific subset indicates that macrophage proliferation in tumors has characteristics typical of self-renewal.

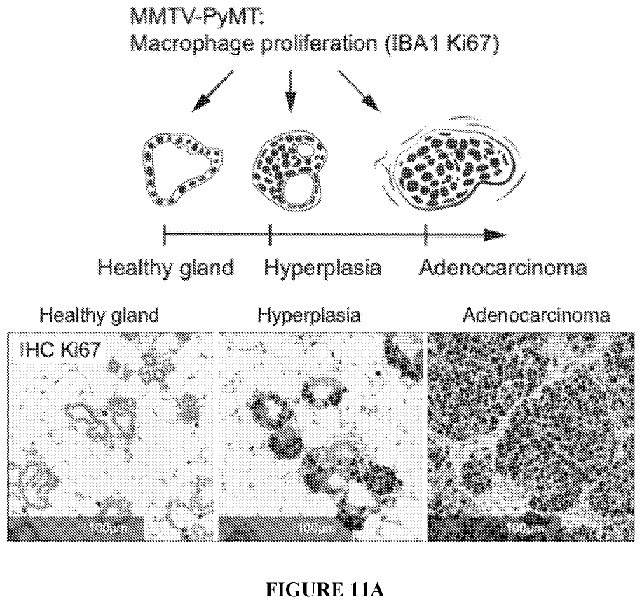

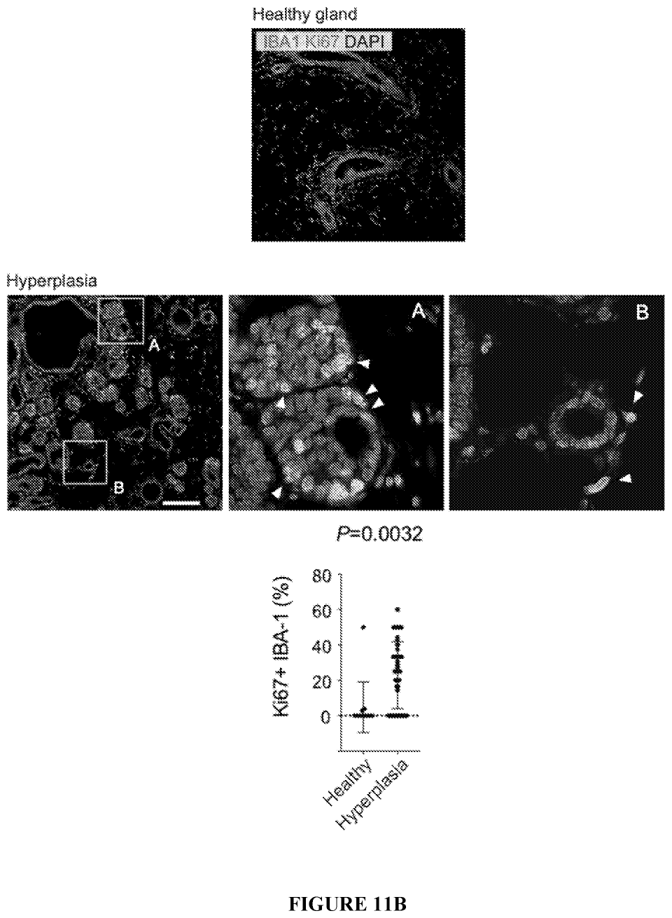

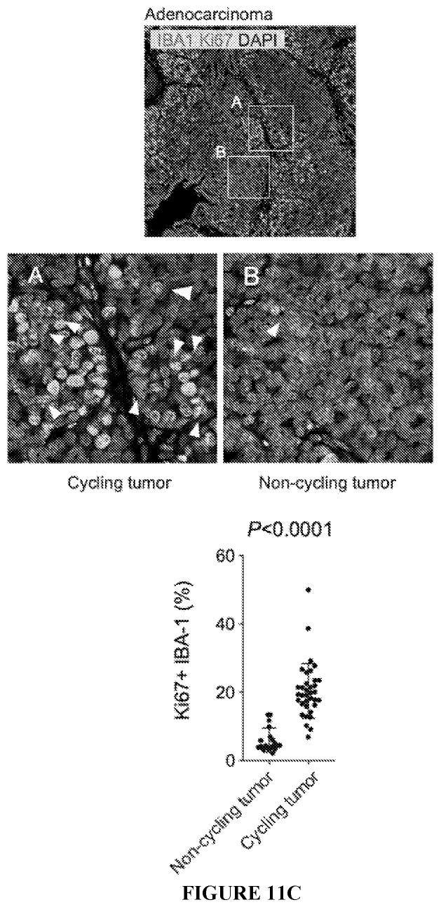

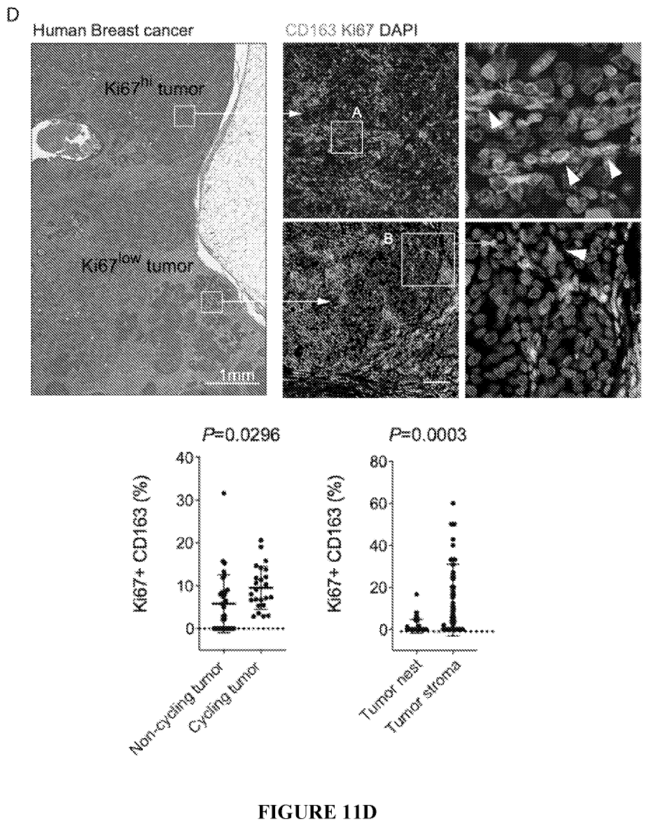

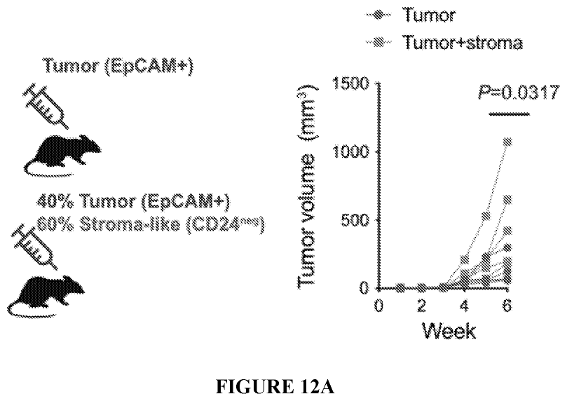

Macrophage long-term accumulation in tumors was previously shown to be unaffected by genetic ablation of monocyte recruitment (Ccr2-KO MMTV-PyMT) (Franklin et al., “The Cellular and Molecular Origin of Tumor-associated Macrophages,” Science 344:921-925 (2014a), which is hereby incorporated by reference in its entirety). Therefore, it was hypothesized that macrophage proliferation would start at the early stages of mammary gland transformation and persist as tumors grow. To address this hypothesis, Ki67 macrophages in mammary tissues from normal, early (hyperplasia), and late (adenocarcinoma) stages of tumor progression were scored in the MMTV-PyMT model (FIG. 11A). In line with a local renewal framework, an early onset of macrophage proliferation (IBA1+Ki67+) was evident already in the pre-cancerous hyperplastic lesions and surrounding stromal areas (22.9%±1.97% mean±SEM) but significantly lower (4.73%±4.13% mean±SEM) in healthy counterparts (FIG. 11B). In late adenocarcinomas, the average number of cycling macrophages persisted in proliferative (Ki67+) tumor areas (20.4%±1.6%) but was significantly lower in non-proliferative (Ki67^{neg}) areas (5.9%±0.80%) (FIG. 11C). The correlation between spatial localization and macrophage proliferations in human specimens of breast cancer was examined. In agreement with the murine data, it was found that Ki67+CD163+ macrophages were preferentially localized in stromal regions (FIG. 11D top panel) but also significantly associated with proliferating tumor areas (Ki67+) (FIG. 11D, bottom panel).

### Example 6

### Identification of Stroma-Associated Macrophages in Mammary Tumors

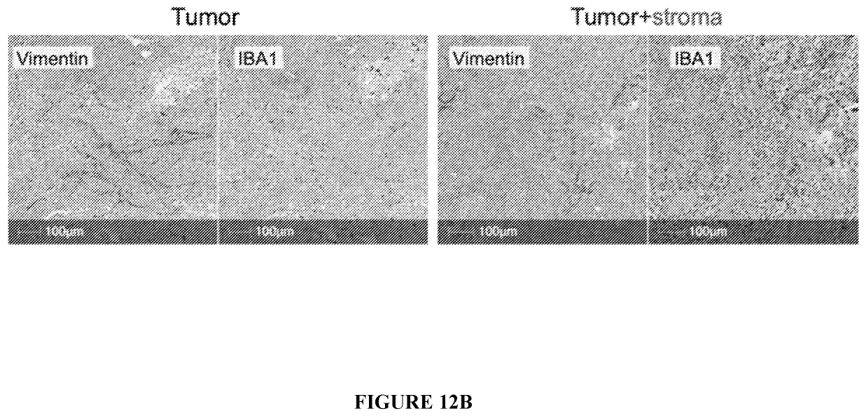

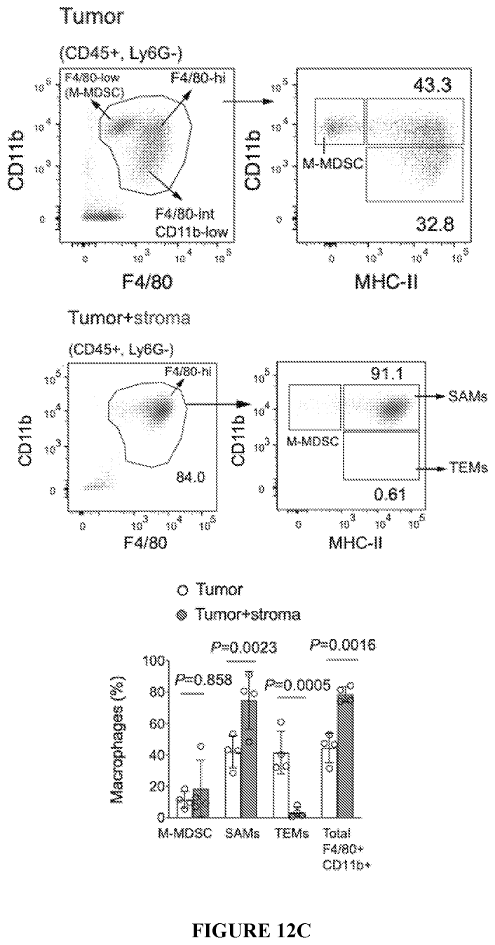

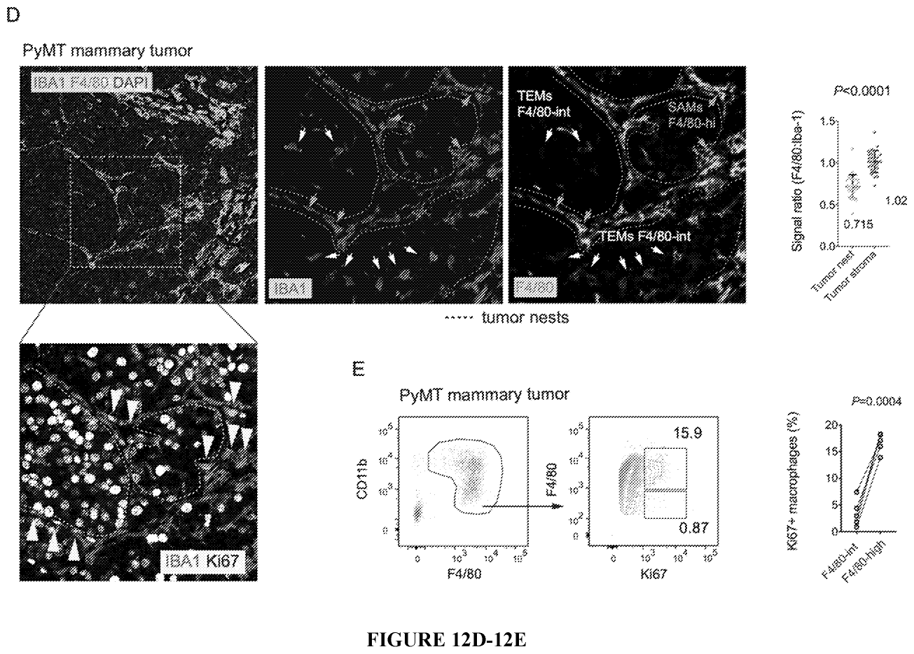

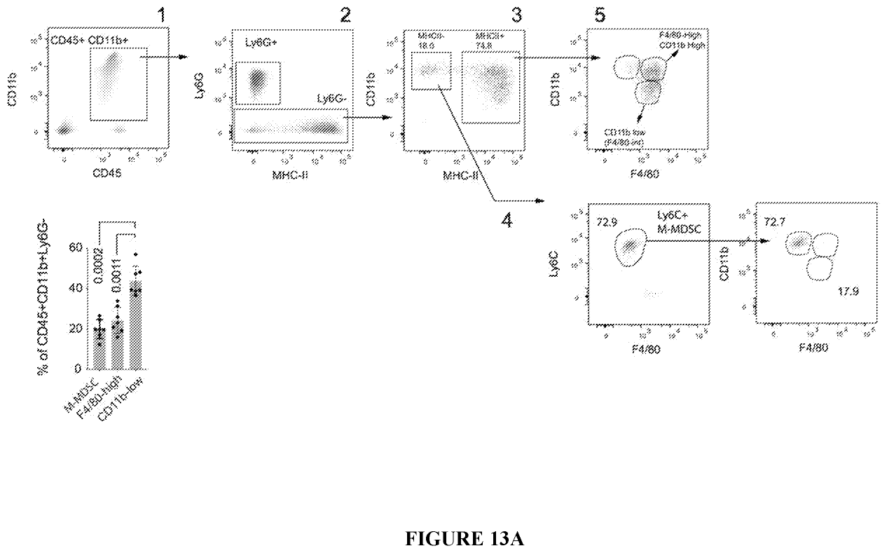

Characterization of the SAM subpopulation in mammary tumors was carried out. It was hypothesized that orthotopic transplantation of tumor epithelial cells enriched with PDGFRA+CD24^{neg} stromal-like cells (40:60% of EpCAM+:CD24^{neg}) would skew the relative proportions of SAMs versus TEMs in these tumors. Accelerated tumor growth was observed in stroma-enriched tumors as previously reported (Orimo and Weinberg, “Stromal Fibroblasts in Cancer: A Novel Tumor-Promoting Cell Type,” Cell Cycle 5:1597-1601 (2006) and Orimo et al., “Stromal Fibroblasts Present in Invasive Human Breast Carcinomas Promote Tumor Growth and Angiogenesis Through Elevated SDF-1/CXCL12 Secretion,” Cell 121:335-348 (2005), which are hereby incorporated by reference in their entirety), in comparison with tumors that originated from tumor epithelial cells-only (FIG. 12A).

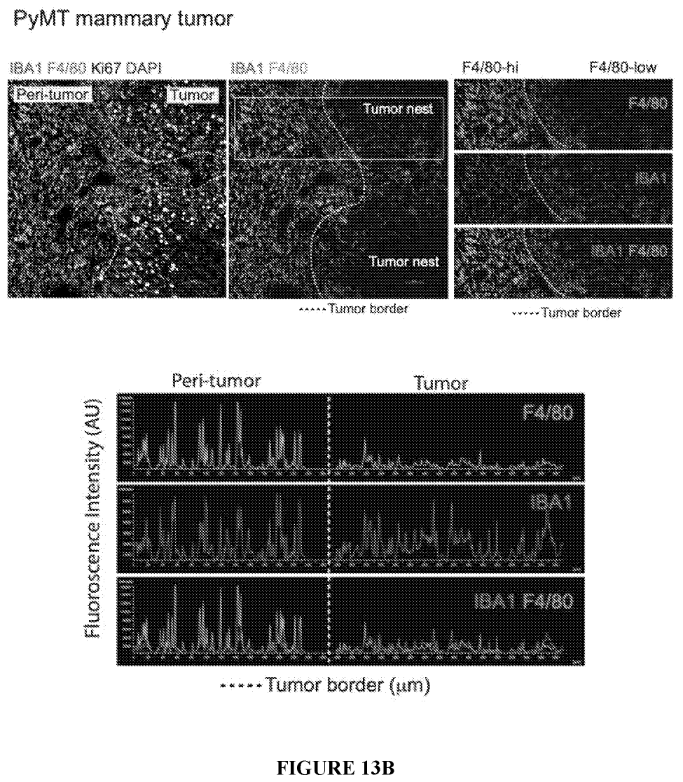

Immunohistochemistry (IHC) staining for vimentin and IBA1 confirmed a substantial enrichment of stromal cells in stroma-enriched tumors and increased accumulation of IBA1+ macrophages (FIG. 12B). To further identify SAMs and TEMs, flow cytometry was used to immunophenotype the myeloid immune landscape of these tumors (FIG. 13A; gating strategy). After exclusion of Ly6G+ granulocytes, the inventors classified a small population (20%±4.7%) of (i) MHC-II^{neg}Ly6C+F4/80^{low} monocytic myeloid-derived suppressor cells (M-MDSC) (Gabrilovich and Nagaraj, “Myeloid-Derived Suppressor Cells as Regulators of the Immune System,” Nat. Rev. Immunol. 9:162-174 (2009), which is hereby incorporated by reference in its entirety), and a major population (74%±1.1%) of MHC-II^{high} macrophages that can be further divided based on CD11b expression: (ii) CD11b^{high}F4/80^{high} or (iii) CD11b^{low}F4/80^{int} (FIG. 12C, left panel). As predicted by the TME modeling, the total frequencies of macrophages in stroma-enriched tumors increased substantially (78.6% vs. 44.5%; P=0.0016, 2-way ANOVA test). Furthermore, significant shifts in the macrophage populations were noticed in the stroma-enriched group without affecting the M-MDSC populations. Whereas the CD11b^{low}F4/80^{int} population was dramatically reduced (from 41.9% to 3.6%, P=0.0023, 2-way ANOVA test), CD11b^{high}F4/80^{high} cells became the predominant population (from 42.3% to 74.9%, P=0.0005, 2-way ANOVA test) suggesting that SAMs in mammary tumors are characterized as CD11b^{high}F4/80^{high}MHC-II^{high} while TEMs are CD11b^{low}F4/80^{Int}MHC-II^{high} (FIG. 12C, right panel).

To further confirm the localization of F4/80^{high} and F4/80^{int} subpopulations in mammary tumors, tumor sections were immunostained with F4/80, IBA1 (pan macrophages marker), and Ki67 to highlight the tumor regions. In agreement with the flow data, tumor nests macrophages (TEMs) displayed a lower signal ratio of F4/80:IBA1, while stromal macrophages were associated with a higher ratio (FIGS. 12D and FIG. 13B) (P=0.0004, two-tailed paired Wilcoxon test) and Ki67 positivity (FIG. 12E). These findings demonstrate that local interactions with neighboring cells may manifest distinct phenotypes in macrophages.

### Example 7

### Spatial Interactions of Macrophages Define Their TME Immunomodulatory Phenotypes

The spatial localization of macrophages in solid tumors confers a significant prognostic value in cancer patients. Clinical data from breast cancer patients showed that infiltration of CD163+ macrophages in tumor stroma (rather than in tumor epithelial nests) correlated with aggressive histopathological characteristics and adverse outcomes (Gwak et al., “Prognostic Value of Tumor-Associated Macrophages According to Histologic Locations and Hormone Receptor Status in Breast Cancer,” PLoS One 10:e0125728 (2015); Medrek et al., “The Presence of Tumor Associated Macrophages in Tumor Stroma as a Prognostic Marker for Breast Cancer Patients,” BMC Cancer 12:306 (2012); Salmi et al., “The Number and Localization of CD68 and CD163 Macrophages in Different Stages of Cutaneous Melanoma,” Melanoma Research 29:237-247 (2019); and Yang et al., “Stromal Infiltration of Tumor-Associated Macrophages Conferring Poor Prognosis of Patients with Basal-Like Breast Carcinoma,” J. Cancer 9:2308-2316 (2018), which are hereby incorporated by reference in their entirety), which further suggests that cellular interactions within the microenvironment may shape their tumor-supportive functions.

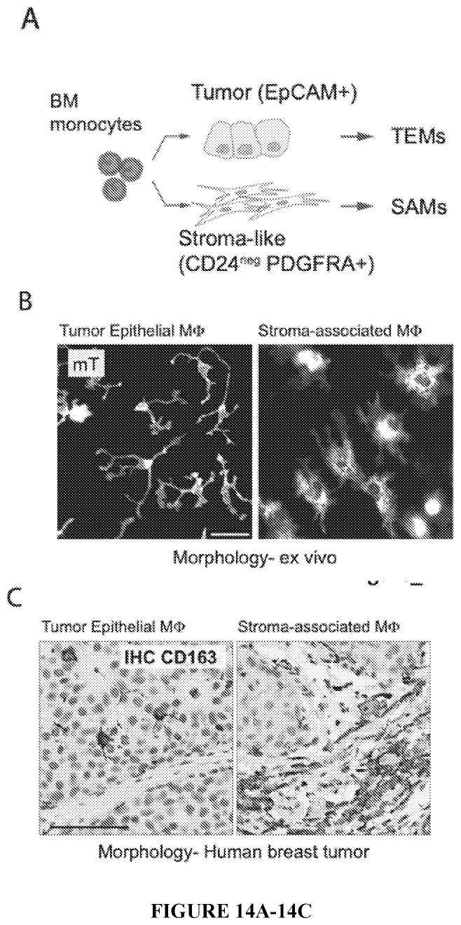

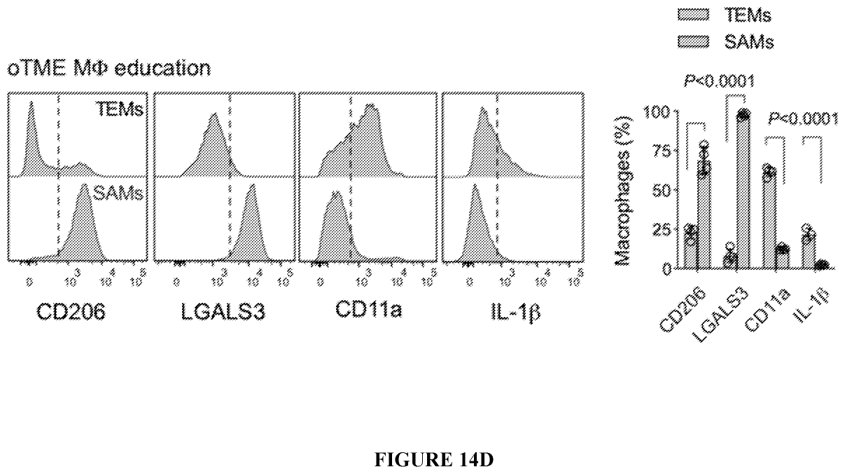

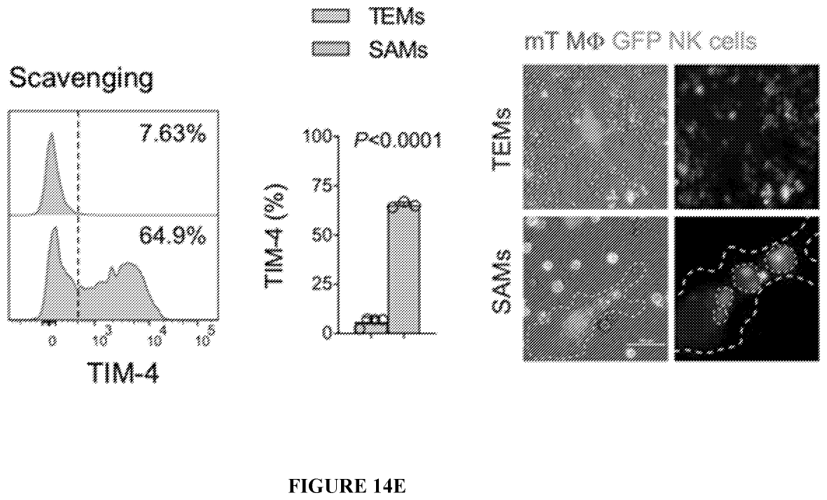

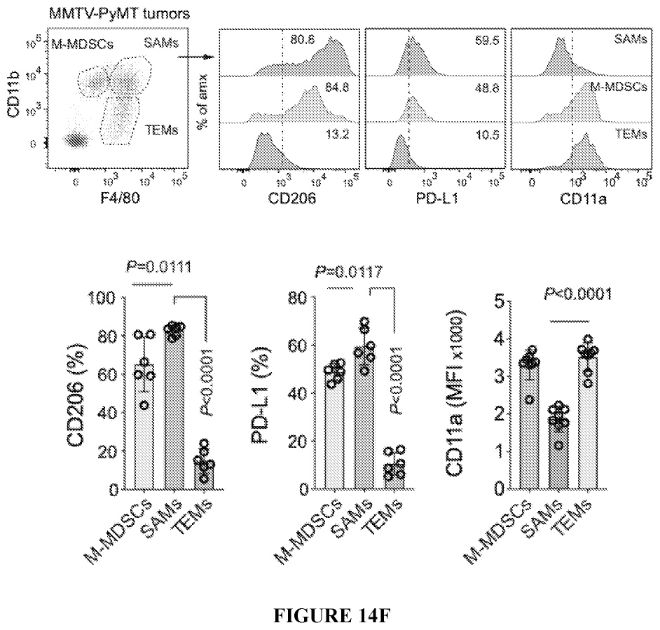

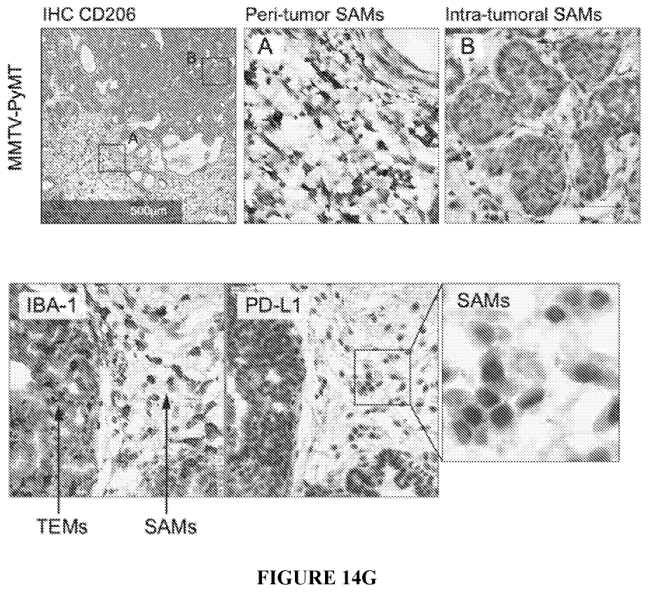

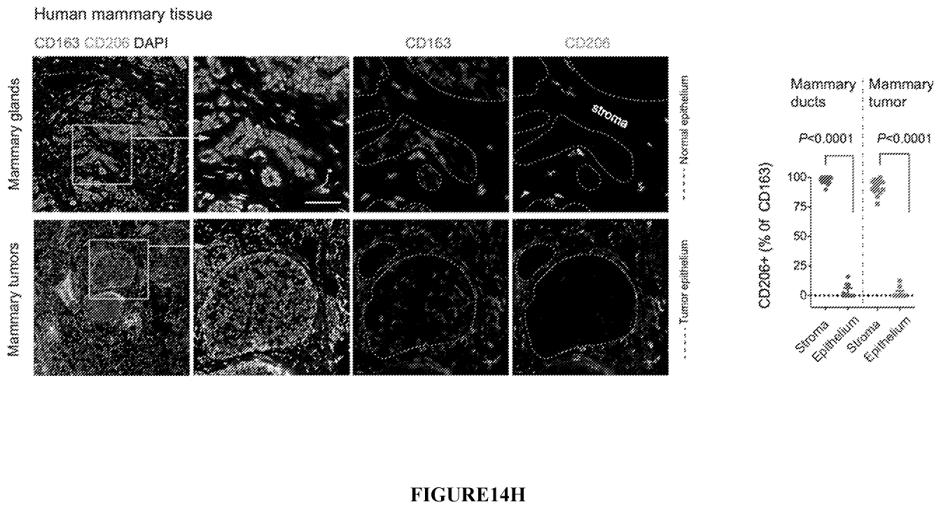

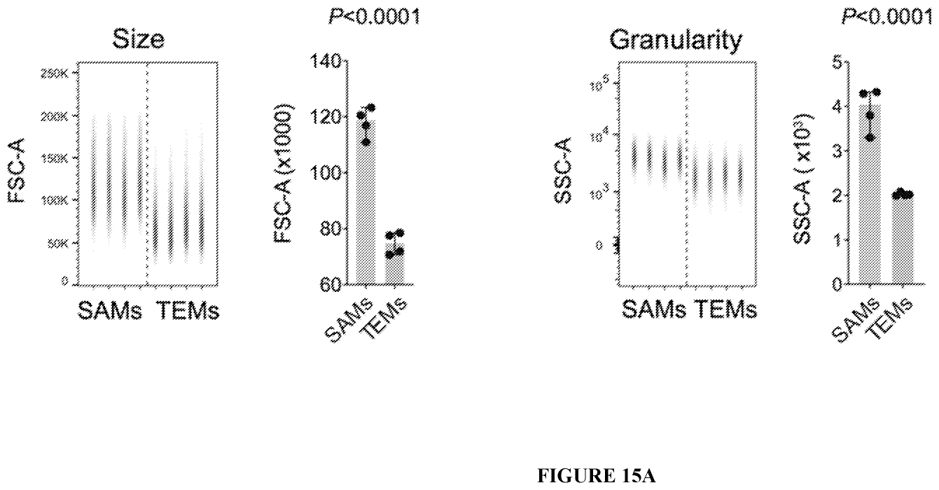

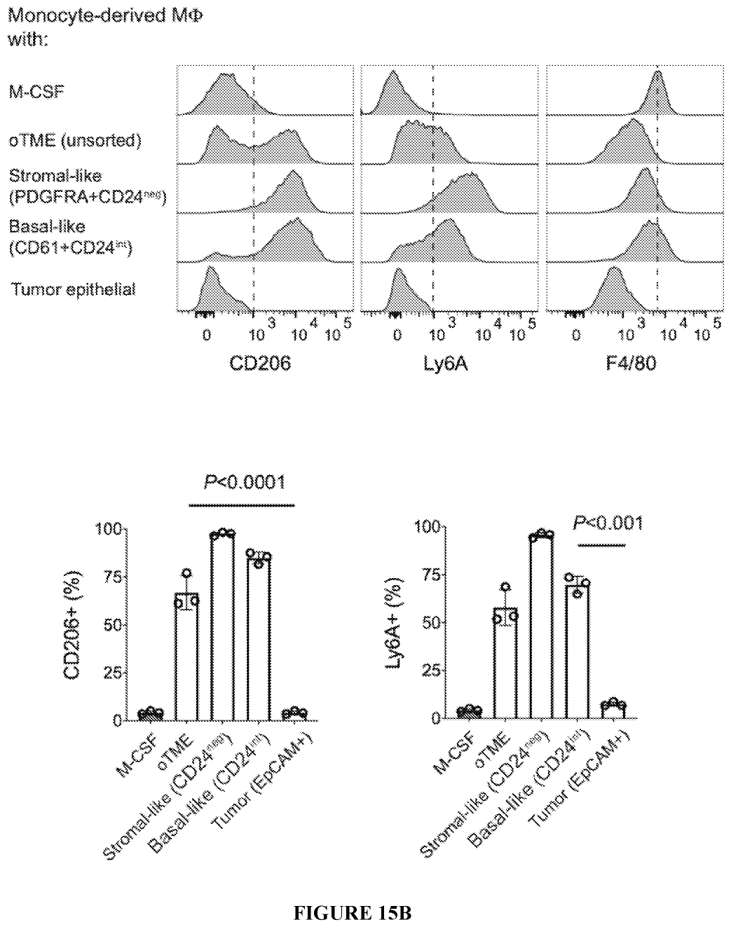

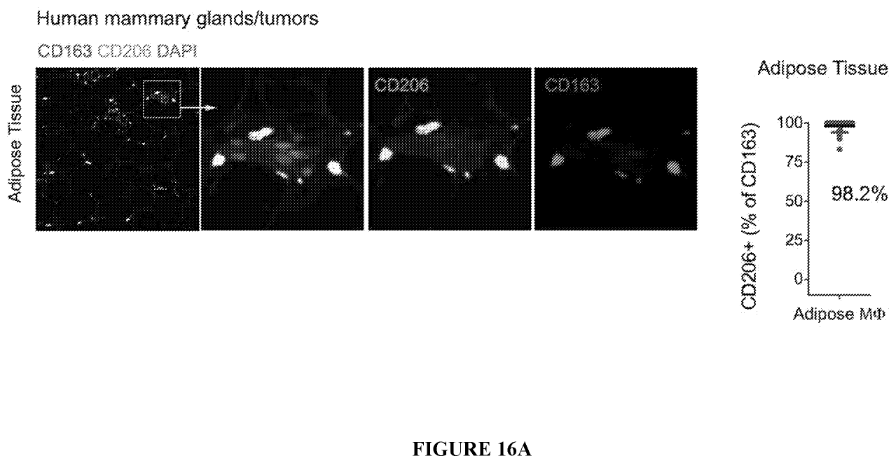

To address this aspect, monocytes were differentiated on tumor epithelial or stromal-like cells and interrogated for functional and immunomodulatory markers (FIG. 14A). First, major morphological differences between monocytes that were differentiated in the presence of tumor epithelial cells or PDGFRA+CD24^{neg} cells was obserced (FIG. 14B). TEM morphology was characterized by extensive and branched dendrites that were localized between epithelial cell junctions. In contrast, SAMs were significantly larger in size (FCS; P<0.0001, two-tailed t-test) and were loaded with phagosomes as reflected by a —100-fold increase in their granularity (SCC; P=0.002, two-tailed t-test) (FIG. 15A). These morphological differences were recapitulated in human breast tumors, demonstrating that CD163+ macrophages exhibited dendritic morphology when localized in tumor nests but were highly granular when localized on tumor stroma (FIG. 14C).

The immunomodulatory and scavenging phenotypes that associate with immune tolerance were further examined (Baghdadi et al., “TIM-4 Glycoprotein-Mediated Degradation of Dying Tumor Cells by Autophagy Leads to Reduced Antigen Presentation and Increased Immune Tolerance,” Immunity 39:1070-1081 (2013); Biswas ad Mantovani, “Macrophage Plasticity and Interaction with Lymphocyte Subsets: Cancer as a Paradigm,” Nat. Immunol. 11:889-896 (2010); Kratochvill et al., “TNF Counterbalances the Emergence of M2 Tumor Macrophages,” Cell Rep. 12:1902-1914 (2015), which are hereby incorporated by reference in their entirety). Immunomodulatory (CD206, LGALS3, PD-L1) and inflammatory markers (CD11a, IL-1b), as well as scavenging mediators (TIM-4) were probed following exposure to GFP-labeled NK cells. Consistent with immunomodulatory impact as a function of spatial localization, the expression of immunosuppressive markers such as CD206, LGALS3, and TIM4 were strongly associated with SAMs, whereas inflammatory markers such as CD11a and IL-1β were associated with TEMs (FIG. 14D-E). Notably, the alteration from inflammatory (CD206^{neg}CD11c^{Hi}F4/80^{int}) to immunosuppressive phenotype (CD206+ CD11^{low}F4/80^{high}) was also evident in cultures with mesenchymal tumor cells (CD61+CD24^{int}EpCAM^{neg}; FIG. 15B), suggesting that epithelial-to-mesenchymal transition (EMT) in tumors can also promote the immunomodulatory characteristics in TME macrophages.

To confirm and visualize the immunomodulatory phenotype of SAMs in vivo, the expression of PD-L1, CD206, and CD11a in macrophages of MMTV-PyMT tumors was analyzed. As shown by flow cytometry, the expression of CD206 and PD-L1 was distinctly compartmentalized (FIG. 14F); CD206 was abundantly detected on SAMs (and M-MDSCs) but was much lower in TEMs (82.9% vs. 14.9%, P=0.001, 2-way ANOVA test). Similarly, PD-L1 was expressed in SAMs (59.47%) but was undetectable in TEMs. This phenotypic compartmentalization was confirmed by IHC staining and CD206+ macrophages were mapped exclusively to tumor stroma or peritumoral areas that were enriched in adipose tissue. However, none of the CD206 positive cells were detected inside tumor nests, consistent with the flow classification of oTME and tumor macrophages (FIG. 14G). The results can explain the earlier observation of downregulation of Mrcl (CD206) in macrophages from tumor of epithelial origin (Franklin et al., “The Cellular and Molecular Origin of Tumor-associated Macrophages,” Science 344:921-925 (2014a), which is hereby incorporated by reference in its entirety) despite that CD206 is being frequently used as a bona fide M2-like marker (Biswas ad Mantovani, “Macrophage Plasticity and Interaction with Lymphocyte Subsets: Cancer as a Paradigm,” Nat. Immunol. 11:889-896 (2010); Murray et al. “Macrophage Activation and Polarization: Nomenclature and Experimental Guidelines,” Immunity 41:14-20 (2014), which are hereby incorporated by reference in their entirety).

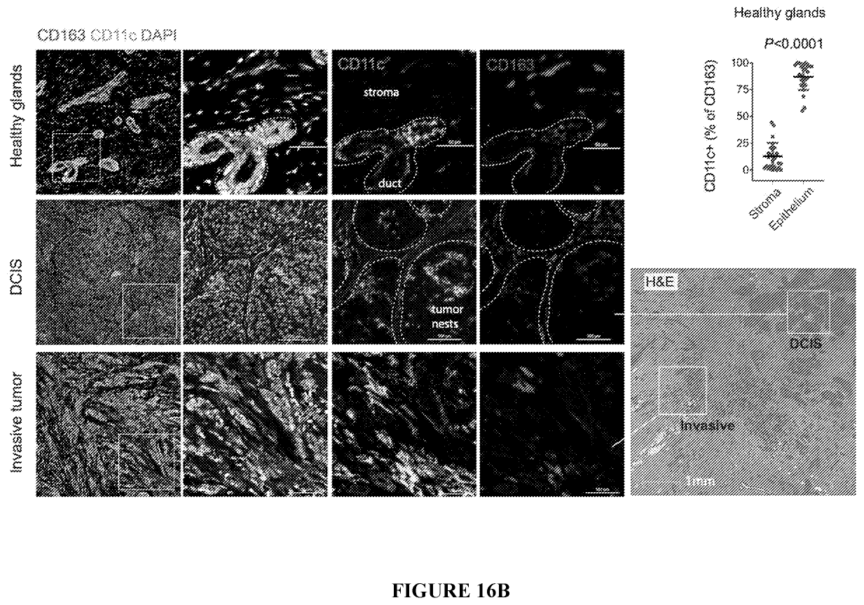

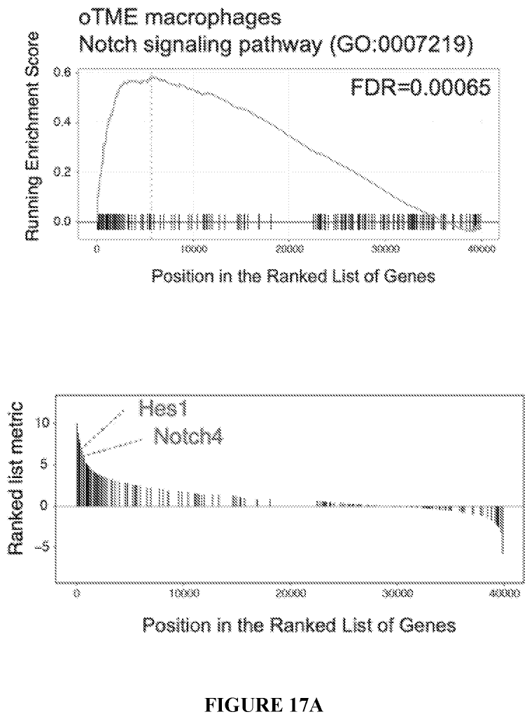

The spatially-defined immunosuppressive phenotype of SAMs was conserved in human breast cancer. As in murine tumors, CD206 was expressed exclusively on CD163+ stromal macrophages of adipose tissue, normal, and tumor stroma regions, but was absent in CD163+ macrophages that associated with normal and tumor epithelia (FIGS. 14H and FIG. 16A). In contrast, the immunostimulatory marker CD11c was predominantly associated with CD163+ macrophages of normal and non-invasive tumor epithelia (DCIS). Notably, when tumors became invasive, CD11c became positive on all macrophages (FIG. 16A). These results suggest that SAMs represent a functionally-distinct population and constitute a major immunosuppressive force in the TME.

### Example 8

### Treatment with NOTCH4 Neutralizing Antibodies Inhibits Macrophage Proliferation and Restrains Tumor Growth In Vivo

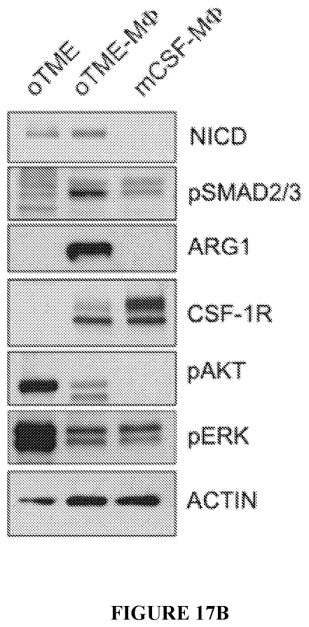

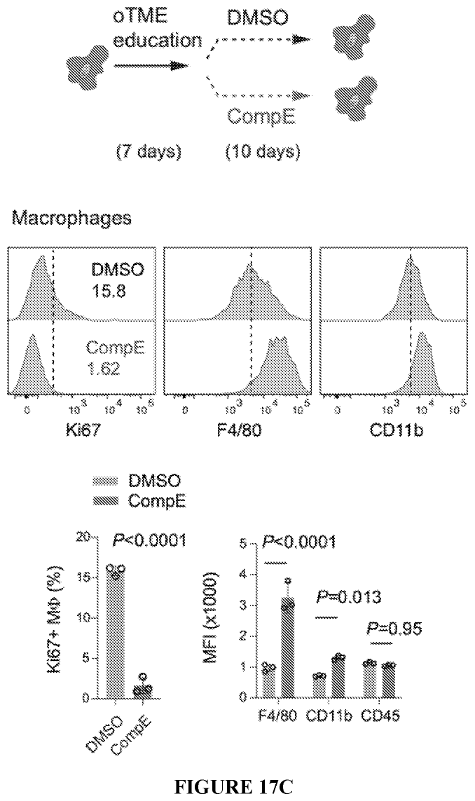

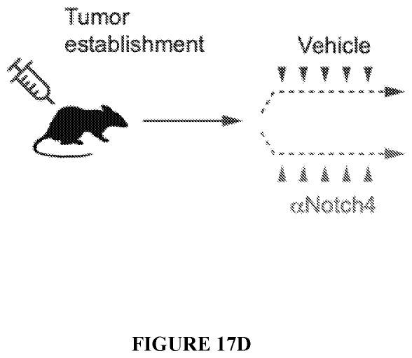

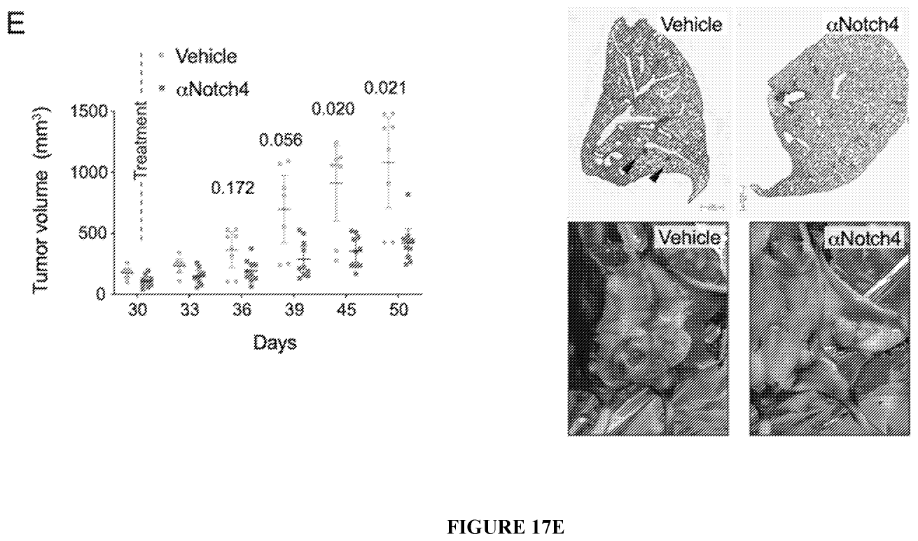

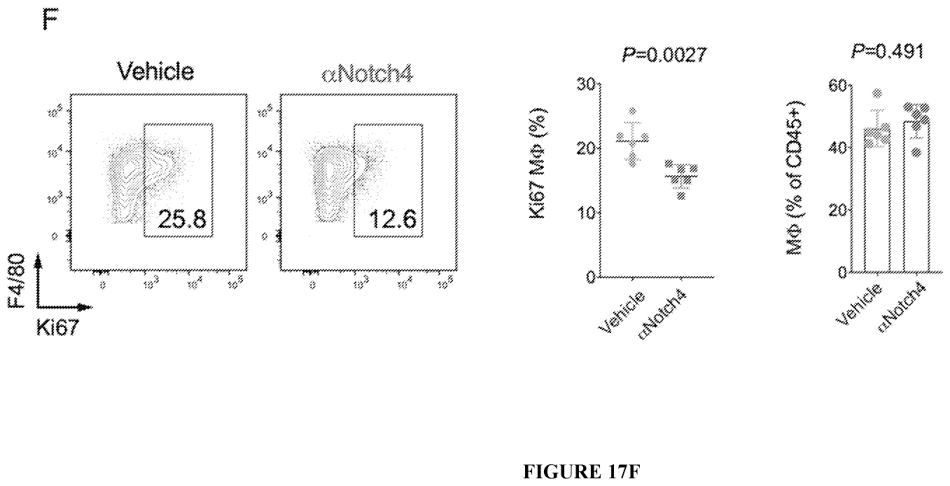

Given that macrophage proliferation was significantly enhanced by cell contact, the relevance of contact-mediated signaling, such as the Notch pathway, to macrophage proliferation was examined. Indeed, activation of Notch signaling in macrophages was previously implicated with inflammation, wound-healing responses, mammary stem cell maintenance, and breast cancer (Palaga et al., “Notch Signaling in Macrophages in the Context of Cancer Immunity,” Front. Immunol. 9:652 (2018); Boniakowski et al., “Macrophage-Mediated Inflammation in Normal and Diabetic Wound Healing,” The Journal of Immunology 199:17-24 (2017); Franklin et al., “The Cellular and Molecular Origin of Tumor-associated Macrophages,” Science 344:921-925 (2014), which are hereby incorporated by reference in their entirety). Gene set enrichment analysis of RNA-seq data from oTME macrophages showed a significant enrichment of the Notch pathway (FIG. 17A; GO:0007219) and upregulation of Notch signaling mediators (Notch4, Dll1, Hesl, Rbpj, Adam17, AdamlO). Notch activation was confirmed in cell lysates from oTME macrophages by probing for the cleaved portion of Notch receptor (NICD) that associated with active PI3K signaling (phospho-AKT) (Kaneda et al., “PI3Kγ is a Molecular Switch that Controls Immune Suppression,” Nature 539:437-442 (2016), which is hereby incorporated by reference in its entirety) and induction of ARG1 (FIG. 17B).

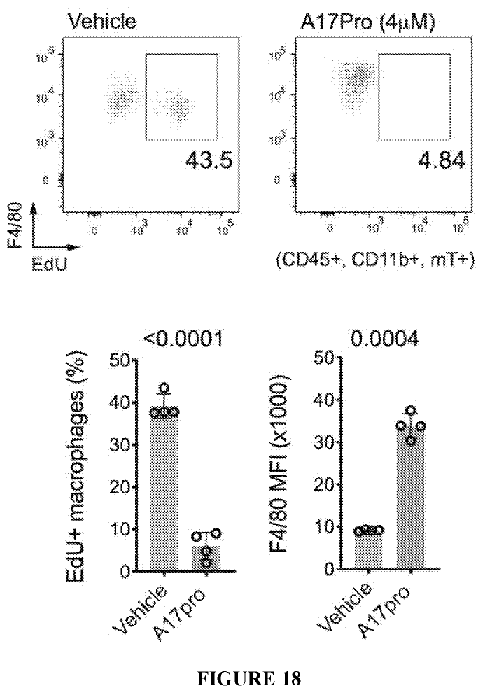

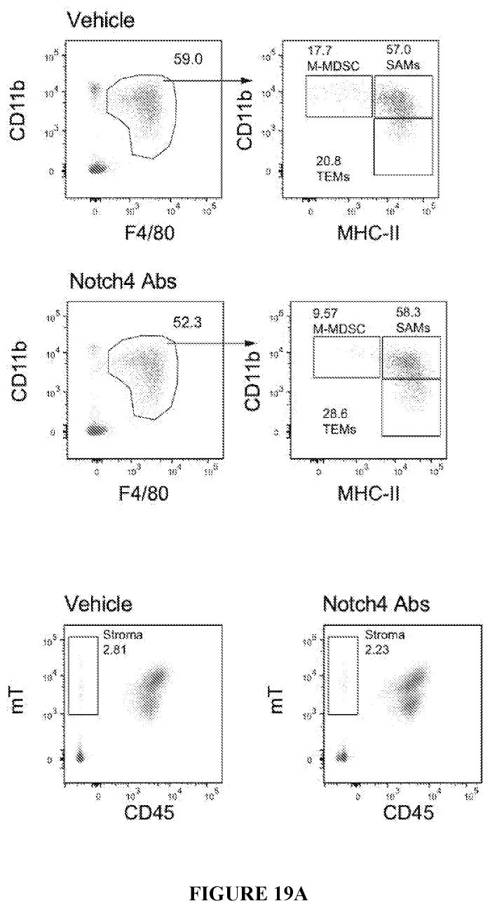

To determine the effect of Notch signaling on macrophage proliferation the inventors scored for EdU incorporation and Ki67 in oTME macrophages following treatment with two protease inhibitors that target Notch activation: (i) metalloproteinase Adaml 7 inhibitors (A17Pro) that inhibit the extracellular cleavage of Notch receptors and macrophage inflammation (Wong et al., “Harnessing the Natural Inhibitory Domain to Control TNFa Converting Enzyme (TACE) Activity In Vivo,” Sci. Rep. 6:35598 (2016), which are hereby incorporated by reference in their entirety), and (ii) γ-secretase protease inhibitors (GSI; Compound-E) that inhibit the subsequent intracellular cleavage of activated Notch receptors. Both treatments resulted in a significant reduction of EdU incorporation (84.8%±3.9% mean±SEM; A17Pro), and in Ki67 positivity (89.6±3.8%; Compound-E) (FIGS. 17C and FIG. 18), and increased expression of F4/80 which is typically down-regulated upon macrophage infiltration into mammary tumors (FIG. 13B). Given that transient type-I interferon activation was important to initiate proliferation in resting macrophages, these results suggest that Notch signaling is required for maintaining macrophage proliferation in TME context.

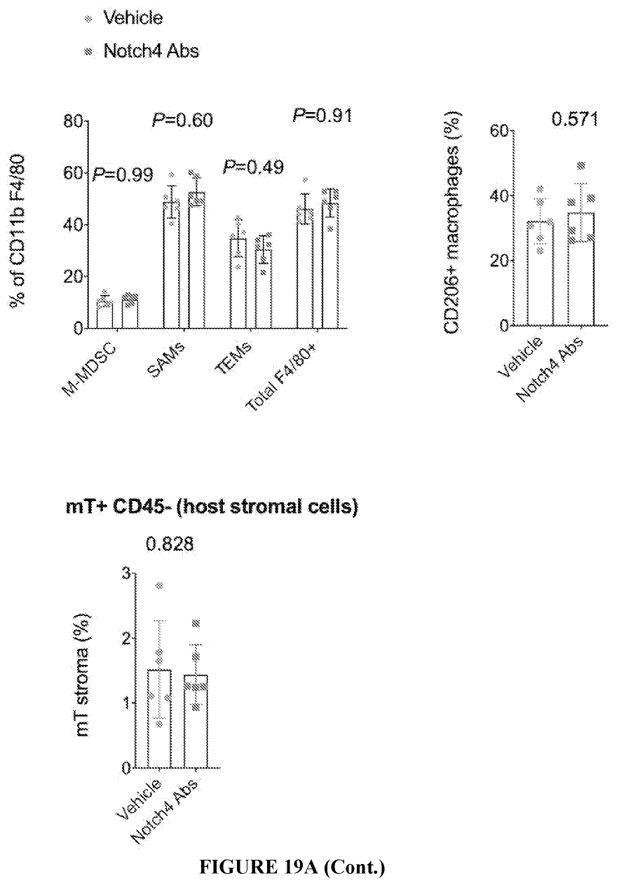

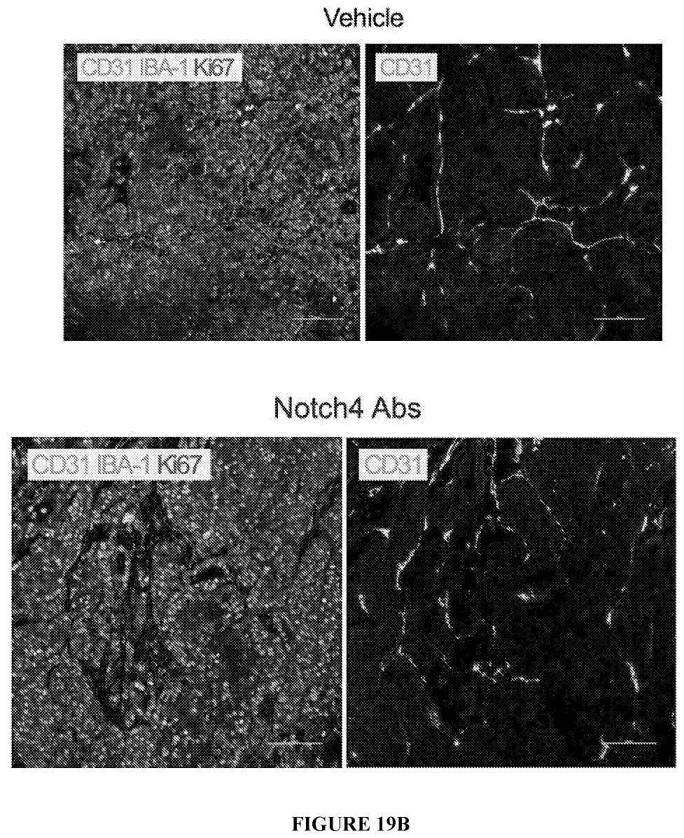

Among the upregulated Notch-related genes, Notch4 was identified as one of the top induced genes in oTME macrophages (FIG. 17A). Therefore, the effects of NOTCH4 neutralization on macrophage proliferation and tumor growth in mice were investigated. Tumor epithelial cells were engrafted orthotopically, and tumors were allowed to establish (˜150 mm³) before treatments were initiated (FIG. 17D; STAR Methods). Treatment with anti-NOTCH4 monoclonal antibodies (15 m/kg body weight, every 72 hrs) resulted in a substantial attenuation of tumor growth and 2.7-fold±0.54 mean±SEM decrease in tumor volume over the experimental trial (FIG. 17E). The cytostatic effect on tumor growth was coupled with a significant reduction in Ki67+ macrophages (FIG. 17F left) without affecting the relative abundance, CD206+ expression (FIG. 19A), or causing adverse effects such as weight loss as typically observed with γ-secretase inhibitor regimens (van Es et al., “Notch/Gamma-Secretase Inhibition Turns Proliferative Cells in Intestinal Crypts and Adenomas into Goblet Cells,” Nature 435:959-963 (2005); Imbimbo, “Therapeutic Potential of Gamma-Secretase Inhibitors and Modulators,” Curr. Top. Med. Chem. 8:54-61 (2008), which are hereby incorporated by reference in their entirety). In addition, IHC staining for CD31 showed no evident effects on tumor vascularization (FIG. 19B) as typically observed following pan macrophage depletion interventions (Bonapace et al., “Cessation of CCL2 Inhibition Accelerates Breast Cancer Metastasis by Promoting Angiogenesis,” Nature 515:130-133 (2014) and Keklikoglou et al., “Periostin Limits Tumor Response to VEGFA Inhibition,” Cell Rep. 22:2530-2540 (2018), which are hereby incorporated by reference in their entirety). Notably, the profound inhibition of tumor growth coupled with selective reduction of Ki67+ macrophages further suggests that the cytostatic effect on mammary tumors following NOTCH4 neutralization is acting directly through inhibition of macrophage self-renewal and highlights Notch4 as a potential therapeutic candidate.

### Discussion of Examples 1-8

In this study, the inventors developed an organotypic TME system to define mechanisms of two fundamental aspects of macrophage biology in mammary tumors: (i) to adopt pro-tumoral phenotypes and (ii) their ability to accumulate through self-renewal. These findings were validated in murine models and primary human breast cancer specimens, demonstrating that the oTME model recapitulates the tumor-stroma-macrophage interactions with a high degree of phenotypic fidelity. First, the inventors leveraged this model's scalability and conducted a phenotypic CRISPR/Cas9 screen in primary macrophages to discover gene targets that disrupt the formation of immunosuppressive macrophages. Using the expression of Arg1 as a surrogate for the M2-like phenotype, known mediators of macrophage education (e.g., Stat3, Marco) were identified in addition to novel targets including Cdk4 and Ptk2b that were essential for adopting the Argl+immunosuppressive phenotype.

The underlying mechanisms that regulate macrophage proliferation in mammary tumors was identified. Although macrophages leave the cell cycle upon differentiation (Aziz et al., “MafB/c-Maf Deficiency Enables Self-renewal of Differentiated Functional Macrophages,” Science 326:867-871 (2009) and Klappacher et al., “An Induced Ets Epressor Complex Regulates Growth Arrest During Terminal Macrophage Differentiation,” Cell 109:169-180 (2002), which are hereby incorporated by reference in their entirety), they are able to proliferate again in response to pathological challenges including inflammatory response, tissue repair, obesity, and infection (Robbins et al., “Local Proliferation Dominates Lesional Macrophage Accumulation in Atherosclerosis,” Nat. Med. 19:1166-1172 (2013); Bosurgi et al., “Macrophage Function in Tissue Repair and Remodeling Requires IL-4 Or IL-13 with Apoptotic Cells,” Science 356:1072-1076 (2017); Minutti et al., “Local Amplifiers of IL-4Rα-mediated Macrophage Activation Promote Repair in Lung and Liver,” Science 356:1076-1080 (2017); Amano et al., “Local Proliferation of Macrophages Contributes to Obesity-Associated Adipose Tissue Inflammation,” Cell Metab. 19:162-171 (2014), which are hereby incorporated by reference in their entirety). To uncover the mechanisms that enable reactivation of cell-cycle in TME macrophages, the inventors performed scRNA-seq time-course analysis and followed the transcriptional changes associated with proliferative and non-proliferative cells. This revealed that macrophages first undergo a transient pro-inflammatory activation, typical of type-I interferons/STING signaling prior to the acquisition of anti-inflammatory phenotype. During this pro-inflammatory phase, activated macrophages engaged the cell-cycle and upregulated Ly6A (Sca-1) that marked a subset of proliferating TME macrophages. Dual EdU/BrdU pulse-chase experiments further revealed an expansion pattern typical of self-renewal, where this cycling subset entered a continuous proliferative mode. Pharmacological inhibition of the early type-I interferons signaling attenuated the onset of macrophage proliferation, suggesting that the transient activation of IFN receptors (IFNAR) was essential for their return into the cell cycle. Interestingly, analogous mechanisms were described during the reactivation of cell cycle in dormant HSCs (Essers et al., “IFNalpha Activates Dormant Haematopoietic Stem Cells in Vivo,” Nature 458:904-908 (2009); Ito et al., “Hematopoietic Stem Cell and Progenitor Defects in Sca-1/Ly-6A-null Mice,” Blood 101:517-523 (2003); Walter et al., “Exit From Dormancy Provokes DNA-Damage-Induced Attrition in Haematopoietic Stem Cells,” Nature 520:549-552 (2015), which are hereby incorporated by reference in their entirety). In response to systemic treatment of IFNα and IFNAR signaling, quiescent HSCs upregulated Ly6A that mediated their return back into the cell cycle.

The expression of Ly6A in TME macrophages revealed informative clues about their spatial localization. The inventors found that Ly6A is a novel marker for macrophages in proximity to stromal fibroblasts (SAMs) and further delineates Ly6A^{high}F4/80^{high}CD11b^{high} as the major proliferative macrophage subset in mammary tumors. Regulated self-renewal of tissue-resident macrophages is critical for their maintenance in healthy tissues, including the mammary glands (Hashimoto et al., “Tissue-Resident Macrophages Self-Maintain Locally Throughout Adult Life With Minimal Contribution From Circulating Monocytes,” Immunity 38:792-804 (2013), which are hereby incorporated by reference in their entirety). Although a substantial portion of TME macrophages can originate from circulating monocytes (Movahedi et al., “Different Tumor Microenvironments Contain Functionally Distinct Subsets of Macrophages Derived From Ly6C(high) Monocytes,” Cancer Research 70:5728-5739 (2010), which is hereby incorporated by reference in its entirety), their abundance in mammary tumors was unaffected by genetic ablation of monocyte recruitment (Franklin et al., “The Cellular and Molecular Origin of Tumor-associated Macrophages,” Science 344:921-925 (2014), which is hereby incorporated by reference in its entirety) further suggesting that mammary tumors hijack their intrinsic ability to self-renew. In agreement with these observations, it was shown that macrophage proliferation began at early stages of mammary gland transformation (hyperplasia) but continues predominantly in proliferative regions of late-stage carcinomas. The inventors validated a similar correlation between macrophage proliferation and tumor cell proliferation in human breast tumor specimens (FIG. 11D), suggesting that Ki67+ macrophages are strong indicators of proliferative tumors. In fact, these observations were further supported by clinical data from breast cancer patients demonstrating that the presence of proliferating macrophages in tumors was robustly associated with poor outcomes and early recurrence, independent of the tumor molecular subtype Campbell et al., “Proliferating Macrophages Associated with High Grade, Hormone Receptor Negative Breast Cancer and Poor Clinical Outcome,” Breast Cancer Res. Treat. 128:703-711 (2011), which is hereby incorporated by reference in its entirety).

Although macrophage proliferation is critically dependent on M-CSF/CSF1R signaling, M-CSF availability was insufficient to license proliferation burst in resting cells (Aziz et al., “MafB/c-Maf Deficiency Enables Self-renewal of Differentiated Functional Macrophages,” Science 326:867-871 (2009), which is hereby incorporated by reference in its entirety), but was significantly enhanced through contact with activated (POSTN+) fibroblasts. Consistent with contact-mediated signaling, it was found that Notch signaling and particularly Notch4 regulate macrophage proliferation in mammary tumors. The inventors demonstrated an effective and significant inhibition of tumor growth in mouse models in response to Notch4 neutralization, coupled with reduction in Ki67+ TME macrophages. Since solid tumors critically rely on abundant presence of macrophages through local renewal, effective disruption of this process resulted in an attenuated expansion of tumors but without depleting the existing macrophages. Therefore, no detectable changes in tumor vascularization (CD31) were observed in Notch4 Abs-treated tumors. This mechanism of action may be clinically important given the fact that pan-macrophage depletion approaches have been associated with a substantial reduction in tumor vasculature (Keklikoglou et al., “Periostin Limits Tumor Response to VEGFA Inhibition,” Cell Rep. 22:2530-2540 (2018); Lobov et al., “WNT7b Mediates Macrophage-Induced Programmed Cell Death in Patterning of the Vasculature,” Nature 437:417-421 (2005); Qian et al., “CCL2 Recruits Inflammatory Monocytes to Facilitate Breast-Tumour Metastasis,” Nature 475:222-225 (2011), which are hereby incorporated by reference in their entirety), and cessation of such interventions led to increased angiogenesis and metastatic resurgence (Bonapace et al., “Cessation of CCL2 Inhibition Accelerates Breast Cancer Metastasis by Promoting Angiogenesis,” Nature 515:130-133 (2014), which is hereby incorporated by reference in its entirety). Therefore, neutralization of Notch4 may represent an effective but also safe strategy for therapeutic intervention in breast cancer patients by targeting macrophage self-renewal.

Phenotypic plasticity is a hallmark of the mononuclear phagocytes including monocytes, macrophages, and dendritic cells (Biswas ad Mantovani, “Macrophage Plasticity and Interaction with Lymphocyte Subsets: Cancer as a Paradigm,” Nat. Immunol. 11:889-896 (2010) and Mantovani et al., “Macrophage Polarization: Tumor-Associated Macrophages as a Paradigm for Polarized M2 Mononuclear Phagocytes,” Trends Immunol. 23:549-555 (2002), which are hereby incorporated by reference in their entirety). Macrophages are particularly susceptible to and shaped by signals in their microenvironment (Gautier et al., “Gene-Expression Profiles and Transcriptional Regulatory Pathways that Underlie the Identity and Diversity of Mouse Tissue Cacrophages,” Nat. Immunol. 13:1118-1128 (2012); Lavin et al., “Tissue-Resident Macrophage Enhancer Landscapes are Shaped by the Local Microenvironment,” Cell 159:1312-1326 (2014); Mass et al., “Specification of Tissue-Resident Macrophages During Organogenesis,” Science 353 (2016), which are hereby incorporated by reference in their entirety). Conventionally, macrophages of solid tumors are broadly termed as “tumor-associated macrophages; TAMs” by virtue of their gross association within solid tumors (Biswas ad Mantovani, “Macrophage Plasticity and Interaction with Lymphocyte Subsets: Cancer as a Paradigm,” Nat. Immunol. 11:889-896 (2010); Gordon, “Alternative Activation of Macrophages,” Nat. Rev. Immunol. 3:23-35 (2003); Wynn et al., “Macrophage Biology in Development, Homeostasis and Disease,” Nature 496:445-455 (2013), which are hereby incorporated by reference in their entirety). However, the inventors' results provide compelling evidence that within the TME, local interactions between macrophages and their neighboring cells such stromal cells (SAMs), or tumor epithelial cells (TEM), give rise to phenotypically and functionally-distinct subpopulations with a significant impact on breast cancer patient survival (Medrek et al., “The Presence of Tumor Associated Macrophages in Tumor Stroma as a Prognostic Marker for Breast Cancer Patients,” BMC Cancer 12:306 (2012) and Yang et al., “Stromal Infiltration of Tumor-Associated Macrophages Conferring Poor Prognosis of Patients with Basal-Like Breast Carcinoma,” J. Cancer 9:2308-2316 (2018), which are hereby incorporated by reference in their entirety). In murine tumor models, SAMs (Sca-1+F4/80^{high}MHC-II^{high}CD11b^{high}) display granular morphology, highly proliferative, scavenging, and uniquely express immunosuppressive markers such as CD206, LGALS3, and PD-L1 (Baghdadi et al., “TIM-4 Glycoprotein-Mediated Degradation of Dying Tumor Cells by Autophagy Leads to Reduced Antigen Presentation and Increased Immune Tolerance,” Immunity 39:1070-1081 (2013); Gordon et al., “PD-1 Expression by Tumour-Associated Macrophages Inhibits Phagocytosis and Tumour Immunity,” Nature 545:495-499 (2017); Kim et al., “Immuno-Subtyping of Breast Cancer Reveals Distinct Myeloid Cell Profiles and Immunotherapy Resistance Mechanisms,” Nat. Cell Biol. 21:1113-1126 (2019); Zhu et al., “CSF1/CSF1R Blockade Reprograms Tumor-Infiltrating Macrophages and Improves Response to T-Cell Checkpoint Immunotherapy in Pancreatic Cancer Models,” Cancer Res. 74:5057-5069 (2014), which are hereby incorporated by reference in their entirety). On the other hand, TEMs (Sca-1^{neg}F4/80^{int}MHC-II^{high}CD11b^{low}) have dendritic-like morphology and express inflammatory markers such as IL-1b, CD11a^{high}, and CD11c^{high}. Importantly, the inventors validated these findings in specimens of human breast cancer and showed a comparable phenotypic compartmentalization in TME macrophages. As predicted by the murine models, the intraepithelial macrophages from normal and malignant tissues were non-proliferative, displayed dendritic morphology, and expressed inflammatory markers (CD163+, CD206^{neg}, CD11c^{high}). On the other hand, macrophages in proximity to stromal regions were highly granular, proliferative and expressed surface markers that associate with immunosuppressive phenotype (CD163^{high}CD206^{high}CD11c^{low}) (Ramos et al., “CD163+ Tumor-Associated Macrophage Accumulation in Breast Cancer Patients Reflects Both Local Differentiation Signals and Systemic Skewing of Monocytes,” Clin Transl Immunology 9:e1108 (2020), which is hereby incorporated by reference in its entirety). Collectively, these findings suggest that within the TME macrophages, the SAM population is a primary source for immunosuppressive signals in the TME. In addition, these findings also provide mechanistic insights into previous clinical data showing that accumulation of CD163+ macrophages in tumor stroma is associated with shorter patient survival and disease recurrence, as compared when accumulated in tumor nests (Medrek et al., “The Presence of Tumor Associated Macrophages in Tumor Stroma as a Prognostic Marker for Breast Cancer Patients,” BMC Cancer 12:306 (2012); Salmi et al., “The Number and Localization of CD68 and CD163 Macrophages in Different Stages of Cutaneous Melanoma,” Melanoma Research 29:237-247 (2019); Yang et al., “Stromal Infiltration of Tumor-Associated Macrophages Conferring Poor Prognosis of Patients with Basal-Like Breast Carcinoma,” J. Cancer 9:2308-2316 (2018), which are hereby incorporated by reference in their entirety).

Collectively, the findings described herein emphasize the therapeutic potential of modeling the tumor-stroma-macrophage interactions using an organotypic TME system to decipher the molecular mechanisms underlying macrophage education and proliferation. Thus, deconvolution cell-cell interactions of macrophages within their microenvironment may pave the way for the next generation of immunotherapies to harness the innate immunity against cancer.

### Example 9

### Modeling Immune Suppression of T cells and NK Cells in TME Culture Sytem

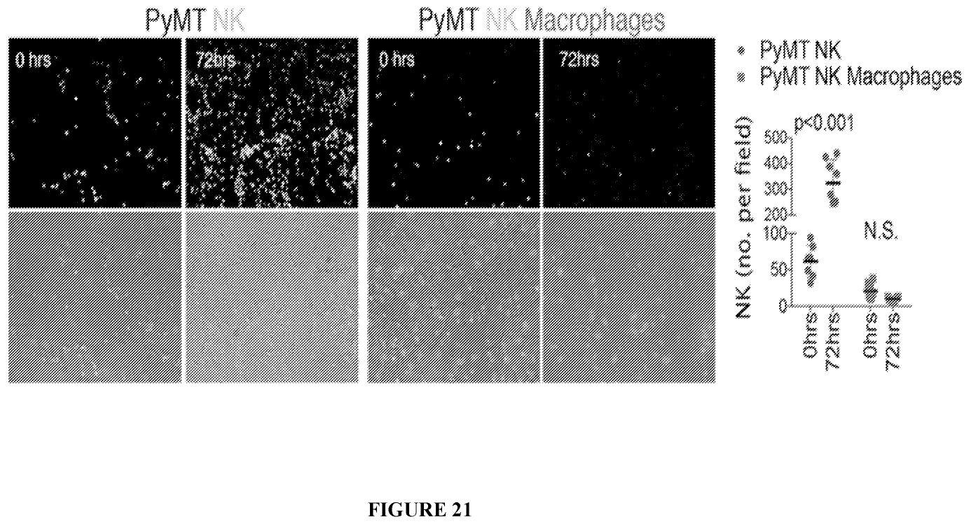

NK cells. The oTME captures the suppressive interactions between NK cells and oTME macrophages and provides functional read-outs for high throughput screens to overcome NK suppression in solid tumors. It was found that in the absence of macrophages, NK cells (spleen-purified) were capable of killing tumor cells and unexpectedly proliferate; however, this response was abrogated by the presence of oTME macrophages (FIG. 21). Notably, the suppression of NK cells occurred only in the presence of tumor cells, closely recapitulating the education process of macrophages in tumors.

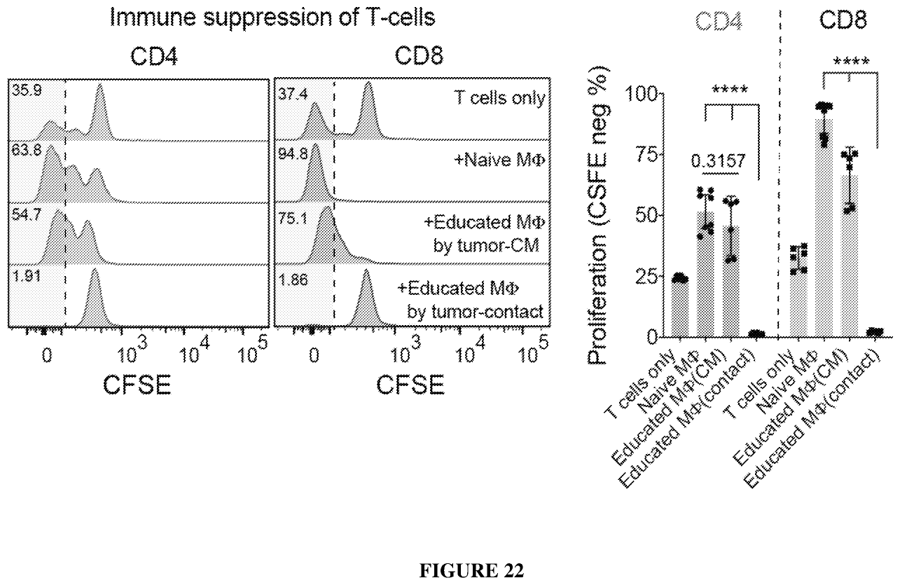

T-cells. Similar to NK cells, oTME macrophages were able to suppress the growth of activated T-cells effectively but only when cell-cell contact was allowed (see FIG. 22). These results suggest that cell-cell interactions rather than oTME-secreted factors mediate immune suppression by macrophages. Using the growth inhibition of T cells as a screen readout, novel mechanisms by which T cells overcome growth suppression in solid tumors can be screened and identified.

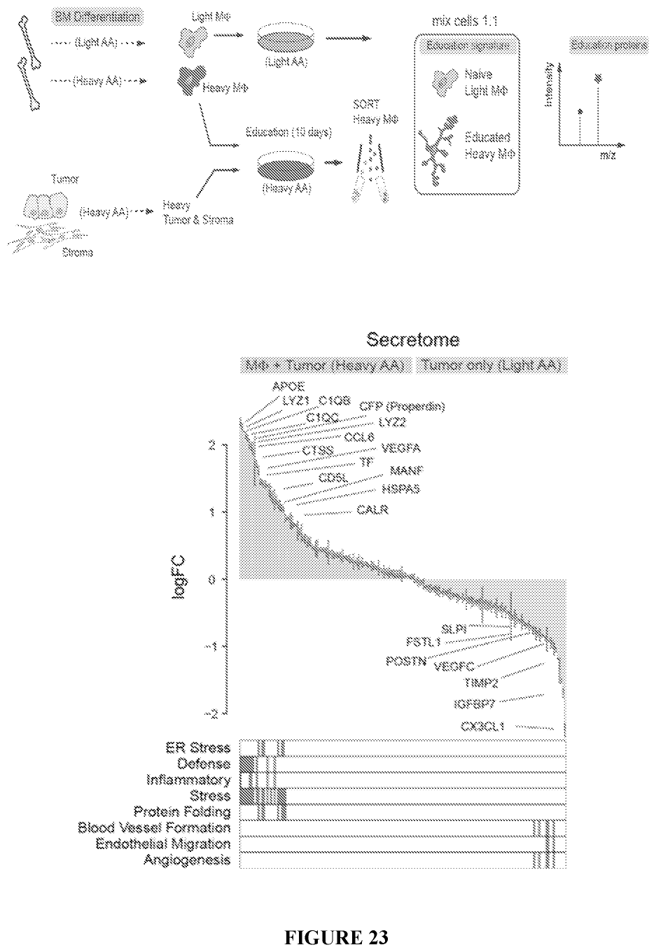

High throughput proteomics of intracellular and secreted proteins. Secreted cytokines and growth factors are critical mediators of immune responses. However, the ability to measure secreted proteins or identify their source in tumor tissues is limited. To overcome this limitation, a protein labeling strategy was developed that relies on heavy amino acid (AA) incorporation by Stable Isotope Labeling by Amino Acids in Cell Culture (SILAC) and enables the identification of secreted proteins and their secreting source by mass spectrometry (MS). For detection of intracellular proteins (FIG. 23, left): cells of interest are first cultured separately either in heavy AA (condition a) or light AA (condition b) containing media. Then, cells are added to oTME, FACS sorted and mixed 1:1 before MS analysis. To analyze secreted proteins from particular cells (FIG. 23, right): cells of interest are first labeled separately in heavy AA and then added to the light AA-labeled oTME culture. Finally, growth media are collected and analyzed by MS.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

1. A method of inhibiting an immunosuppressive phenotype in a population of macrophages, said method comprising:
administering to the population of macrophages, an agent selected from a cyclin-dependent kinase 4 (Cdk4) inhibitor, tumor necrosis factor related apoptosis-inducing ligand receptor 2 (TRAIL-R2) inhibitor, a protein tyrosine kinase 2 beta (Ptk2b) inhibitor, and combinations thereof under conditions effective to inhibit the immunosuppressive phenotype in the population of macrophages.

2. The method of claim 1, wherein the agent is a Cdk4 inhibitor selected from the group consisting of palbociclib (6-acetyl-8-cyclopentyl-5-methyl-2-[(5-piperazin-1-ylpyridin-2-yl)amino]pyrido[2,3-d]pyrimidin-7-one), ribociclib (7-cyclopentyl-N,N-dimethyl-2-[(5-piperazin-1-ylpyridin-2-yl)amino]pyrrolo[2,3-d]pyrimidine-6-carboxamide), abemaciclib (N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine), voruciclib (2-[2-chloro-4-(trifluoromethyl)phenyl]-5,7-dihydroxy-8-[(2R,3S)-2-(hydroxymethyl)-1-methylpyrrolidin-3-yl]chromen-4-one), and trilaciclib (4-[[5-(4-methylpiperazin-1-yl)pyridin-2-yl]amino]spiro[1,3,5,11-tetrazatricyclo[7.4.0.02,7]trideca-2,4,6,8-tetraene-13,1′-cyclohexane]-10-one).

3. The method of claim 1, wherein the agent is a Ptk2B inhibitor selected from the group consisting of PF-00562271 (N-methyl-N-[3-[[[2-[(2-oxo-1,3-dihydroindo1-5-yl)amino]-5-(trifluoromethyl)-4-pyrimidinyl]amino]methyl]-2-pyridinyl]methanesulfonamide is a member of indoles), conteltinib (2-[[2-[2-methoxy-4-[4-(4-methylpiperazin-1-yl)piperidin-1-yl]anilino]-6,7-dihydro-5H-pyrrolo[2,3-d]pyrimidin-4-yl]amino]-N-propan-2-ylbenzenesulfonamide), and NVP-TAE226 (2-[[5-chloro-2-(2-methoxy-4-morpholin-4-ylanilino)pyrimidin-4-yl]amino]-N-methylbenzamide)

4. The method of claim 1, wherein the population of macrophages comprises macrophages having an M2 phenotype.

5. The method of any one of claims 1-4, wherein said administering is carried out in vivo to a subject having cancer, said method further comprising:
selecting a subject having a cold tumor, wherein said administering is carried out under conditions effective to induce an immunomodulatory phenotype in macrophage populations surrounding the cold tumor.

6. The method of claim 5, wherein the method further comprises:
administering to the selected subject a checkpoint inhibitor in combination with the Cdk4 inhibitor, TRAIL-R2 inhibitor, Ptk2b inhibitor, Notch-4 inhibitor.

7. The method of claim 6, wherein the checkpoint inhibitor is selected from the group consisting of a programmed death-ligand 1 (PD-L1) inhibitor, a programmed cell death protein 1 (PD-1) inhibitor, a cytotoxic T-lymphocyte-associated protein 4 (CTLA-4) inhibitor, and combinations thereof.

8. The method of claim 7, wherein the checkpoint inhibitor is a PD-1 inhibitor selected from Pembrolizumab, Nivolumab, Pidilizumab, and Cemiplimab

9. The method of claim 7, wherein the checkpoint inhibitor is a PD-L1 inhibitor selected from Atezolizumab, Avelumab, Durvalumab.

10. The method of claim 7, wherein the checkpoint inhibitor is the CTLA-4 inhibitor Ipilimumab.

11. The method of claim 5, wherein the method further comprises:
administering to the selected subject a pro-inflammatory agent in combination with the Cdk4 inhibitor, TRAIL-R2 inhibitor, or Ptk2b inhibitor

12. The method of claim 11, wherein the pro-inflammatory agent is selected from the group consisting of GM-CSF, an OX40 activation antibody, and a TREM2 blocking antibody.

13. The method of claim 5, wherein the cold tumor is selected from the group consisting of a breast tumor, pancreatic tumor, ovarian tumor, prostate tumor, colon tumor, solid tumor, glioma, myeloma, liver tumor, and kidney tumor.

14. A method of inhibiting macrophage proliferation in a population of cells comprising macrophages, said method comprising:
administering a Notch-4 inhibitor to the population of cells under conditions effective to inhibit macrophage proliferation in said population of cells.

15. The method of claim 1 or claim 14, wherein the Notch-4 inhibitor is an anti-Notch-4 antibody or binding fragment thereof.

16. A method of treating a tumor in a subject, said method comprising:
administering, to a subject having a tumor, a Notch-4 inhibitor, wherein said administering induces an anti-tumor immune response in the subject.

17. The method of claim 16, wherein the Notch-4 inhibitor is an anti-Notch-4 antibody or binding fragment thereof.

18. The method of claim 16 or claim 17, wherein the tumor is selected from the group consisting of a breast tumor, pancreatic tumor, ovarian tumor, prostate tumor, lung tumor, colon tumor, solid tumor, glioma, melanoma, myeloma, liver tumor, and kidney tumor.

19. The method of any one of claims 16-18, wherein the tumor is a cold tumor.

20. The method of any one of claims 16-19, wherein the method further comprises administering to the selected subject a checkpoint inhibitor in combination with said Notch-4 inhibitor.

21. The method of claim 20, wherein the checkpoint inhibitor is selected from the group consisting of a PD-L1 inhibitor, a PD-1 inhibitor, a CTLA-4 inhibitor, and combinations thereof.

22. The method of claim 20, wherein the checkpoint inhibitor is a PD-1 inhibitor selected from Pembrolizumab, Nivolumab, Pidilizumab, and Cemiplimab.

23. The method of claim 20, wherein the checkpoint inhibitor is a PD-L1 inhibitor selected from Atezolizumab, Avelumab, Durvalumab.

24. The method of claim 20, wherein the checkpoint inhibitor is the CTLA-4 inhibitor Ipilimumab.

25. The method of any one of claims 16-19, wherein the method further comprises:
administering to the selected subject a pro-inflammatory agent in combination with the Notch-4 inhibitor.

26. The method of claim 25, wherein the pro-inflammatory agent is selected from the group consisting of GM-CSF, an OX40 activation antibody, and a TREM2 blocking antibody.

27. A combination therapeutic comprising:
a Notch-4 inhibitor and
a checkpoint inhibitor.

28. The combination therapeutic of claim 27, wherein the Notch-4 inhibitor is an anti-Notch-4 antibody or binding fragment thereof.

29. The combination therapeutic of claim 27, wherein the checkpoint inhibitor is selected from the group consisting of a PD-L1 inhibitor, a PD-1 inhibitor, a CTLA-4 inhibitor, and combinations thereof

30. The combination therapeutic of claim 27, wherein the checkpoint inhibitor is a PD-1 inhibitor selected from Pembrolizumab, Nivolumab, Pidilizumab, and Cemiplimab.

31. The combination therapeutic of claim 27, wherein the checkpoint inhibitor is a PD-L1 inhibitor selected from Atezolizumab, Avelumab, Durvalumab.

32. The combination therapeutic of claim 27, wherein the checkpoint inhibitor is the CTLA-4 inhibitor Ipilimumab.

33. A combination therapeutic comprising:
a Notch-4 inhibitor and
a pro-inflammatory agent.

34. The combination therapeutic of claim 33, wherein the pro-inflammatory agent is selected from the group consisting of GM-C SF, an OX40 activation antibody, and a TREM2 blocking antibody.

35. An in vitro organotypic tumor microenvironment model (TME) culture system, said system comprising:
an isolated population of cells, said population comprising tumor epithelial cells, mesenchymal stromal cells, and fibroblasts.

36. The culture system of claim 35, wherein the fibroblasts are immortalized.

37. The culture system of claim 35, wherein the population of tumor epithelial cells, fibroblasts, and mesenchymal stromal cells are derived from a tumor selected from the group consisting of a breast tumor, pancreatic tumor, ovarian tumor, prostate tumor, lung tumor, colon tumor, solid tumor, glioma, melanoma, myeloma, liver tumor, and kidney tumor.

38. The culture system of any one of claims 35-37 further comprising:
one or more cell types selected from the group consisting of macrophages, endothelial cells, T cell, NK cells, dendritic cells, and combinations thereof.

39. The culture system any one of claims 35-38, wherein the population of cells is a syngeneic population of cells.

40. The culture system of any one of claims 35-39, wherein the population of cells are primary cells.

41. The culture system of any one of claims 35-40, wherein the population of cells is a population of human cells.

42. The culture system of any one of claims 35-40, wherein the population of cells is a population of murine cells.

43. The culture system of claim 35, wherein the population of tumor epithelial cells and mesenchymal cells are derived from a breast tumor, and said tumor epithelial cells are characterized by EpCAM +/CD49^{high}/CD24^{high}/CD61⁻ expression.

44. A method of identifying a candidate compound capable of modulating macrophage immunosuppressive phenotype in a tumor environment, said method comprising:
providing the organotypic tumor microenvironment model (TME) culture system of any one of claims 35-43, wherein said system comprises macrophages;
administering the candidate compound to the culture system;
assessing one or more markers of macrophage immunosuppressive phenotype in the culture systems before and after said administering; and
identifying a candidate compound as one that is capable of modulating macrophage immunosuppressive phenotype in the tumor environment based on said assessing.

45. A method of identifying a candidate compound capable of modulating NK cell activity in a tumor environment, said method comprising:
providing the organotypic tumor microenvironment model (TME) culture system of any one of claims 35-43, wherein said system further comprises NK cells;
administering the candidate compound to the culture system;
assessing one or more markers of NK cell activity in the culture systems before and after said administering; and
identifying a candidate compound as one that is capable of modulating NK cell activity in the tumor environment based on said assessing.

46. A method of identifying a candidate compound capable of modulating T cell activity in a tumor environment, said method comprising:
providing the organotypic tumor microenvironment model (TME) culture system of any one of claims 35-43, wherein said system further comprises T cells;
administering the candidate compound to the culture system;
assessing one or more markers of T cell activity in the culture systems before and after said administering; and
identifying a candidate compound as one that is capable of modulating T cell activity in the tumor environment based on said assessing.
